(19) United States
(12) Patent Application Publication
(10) Pub. No.: US 2003/0017150 A1
(43) Pub. Date: Jan. 23, 2003

(54) Chronic obstructive pulmonary disease-related immunglobulin derived proteins, compositions, methods and uses

(76) Inventor: Theodore J. Torphy, Bryn Mawr, PA (US)

Correspondence Address: AUDLEY A. CIAMPORCERO JR., JOHNSON & JOHNSON, ONE JOHNSON & JOHNSON PLAZA, NEW BRUNSWICK, NJ, 08933-7003, US

(21) Appl. No.: 10/099,007

(22) Filed: Mar. 14, 2002

Related U.S. Application Data
(60) Provisional application No. 60/275,652, filed on Mar. 14, 2001.

(51) Int. Cl. A01K 67/027; A61K 39/395; C07K 16/42; C07K 16/40

(52) U.S. Cl. 424/131.1; 800/14; 530/387.2; 530/388.26

(57) ABSTRACT

The present invention relates to at least one novel COPD-related human Ig derived protein or specified portion or variant, including isolated nucleic acids that encode at least one COPD-related Ig derived protein or specified portion or variant, COPD-related Ig derived protein or specified portion or variants, vectors, host cells, transgenic animals or plants, and methods of making and using thereof, including therapeutic compositions, methods and devices.

### BACKGROUND OF THE INVENTION

1. Field of the Invention

The present invention relates to human Ig derived proteins (Ig derived proteins), specified portions or variants specific for at least one Chronic Obstructive Pulmonary Disease Related (COPD-related) protein or fragment, COPD-related immunoglobulin derived protein encoding and complementary nucleic acids, host cells, and methods of making and using thereof, including therapeutic formulations, administration and devices.

2. Related Art

Chronic obstructive pulmonary disease (COPD) is persistent obstruction of the airways caused by emphysema or chronic bronchitis. Emphysema is an enlargement of the tiny air sacs of the lungs (alveoli) and the destruction of their walls. Chronic bronchitis is a persistent chronic cough that produces sputum and is not due to a medically discernible cause such as lung cancer. In chronic bronchitis, bronchial glands are enlarged, causing excess secretion of mucus.

There are two causes for the airflow obstruction in COPD. The first is emphysema.

Normally, the clusters of alveoli connected to the small airways (bronchioles) provide a fairly rigid structure and hold the airways open. In emphysema, however, the alveolar walls are destroyed, so the bronchioles lose their structural support. Thus, the bronchioles collapse when air is exhaled. In emphysema, therefore, the airflow narrowing is structural and permanent. The second cause of airflow obstruction is inflammation of the small airways in chronic bronchitis. There is scarring of their walls, swelling of their lining, partial obstruction of their passages by mucus, and spasm of smooth muscle. The swelling, mucus obstruction, and smooth muscle spasm can vary in severity from time to time and may improve in response to bronchodilator drugs. This component of the airflow obstruction is partially reversible.

In the United States, about 14 million people suffer from chronic obstructive pulmonary disease. It's second only to heart disease as a cause of disability that makes people stop working, and it's the fourth most common cause of death. More than 95 percent of all deaths from chronic obstructive pulmonary disease occur in people over age 55. It affects men more frequently than women and is more often fatal in men. It's also fatal more often in whites than in nonwhites and in blue-collar workers than in white-collar workers.

Chronic obstructive pulmonary disease appears more frequently in some families, so there may be an inherited tendency. Working in an environment polluted by chemical fumes or nonhazardous dust may increase the risk of chronic obstructive pulmonary disease. However, smoking increases the risk much more than a person's occupation. About 10 to 15 percent of smokers develop chronic obstructive pulmonary disease. Pipe and cigar smokers develop it more often than nonsmokers but not as often as cigarette smokers. Cigarette smokers have higher death rates from chronic bronchitis and empohysema than nonsmokers. With age, cigarette smokers lose lung function much more rapidly than nonsmokers. The more cigarettes a person smokes, the greater the loss of function. Causes. Irritants cause inflammation of the alveoli. If such inflammation is long-standing, permanent damage may result. White blood cells collect in inflamed alveoli and release enzymes (especially neutrophil elastase) that damage connective tissue in the walls of the alveoli. Smoking further impairs the lung's defenses by damaging the tiny hairlike cells (cilia) lining the airways that normally carry mucus toward the mouth and help expel toxic substances. The body produces a protein, called alpha1-antitrypsin, whose main role is to prevent neutrophil elastase from damaging the alveoli. In a rare hereditary condition, there's little or no alpha1-antitrypsin in the body, so emphysema develops by early middle age, especially in smokers.

All forms of COPD cause air to become trapped in the lungs. The number of capillaries in the walls of the alveoli decreases. These abnormalities impair the exchange of oxygen and carbon dioxide between the alveoli and the blood. In the earlier phases of the disease, blood oxygen levels are decreased, but carbon dioxide levels remain normal. In the later stages, carbon dioxide levels are elevated, and blood oxygen levels fall even further.

Symptoms. The earliest symptom of COPD, which may appear after as little as 5 to 10 years of smoking, is a cough and the raising of mucus, most commonly on arising. The cough is generally mild and is often dismissed as a “normal” smoker's cough, although of course, it is not normal. There is often a tendency for head colds to go down into the chest. During chest colds, sputum often becomes yellow or green because of pus in the sputum. As the years go by, these chest illnesses may become more frequent. They may be accompanied by wheezing, which is often more evident to family members than to the patient.

Around age 60, shortness of breath on effort often appears and is slowly progressive. Ultimately, the patient has shortness of breath on activities of daily living, such as toileting, washing, dressing, and preparing food. About one third of patients experience severe weight loss, which is due in part at least to worsening shortness of breath after eating. Swelling of the legs often develops, which may be due to heart failure. In the late stages of the disease, a chest illness that might have been easily tolerated early in the course of the disease may cause severe shortness of breath at rest, an indication of acute respitory failure.

Diagnosis. In mild COPD, a doctor may find nothing abnormal during a physical examination except for a few wheezes heard through the stethoscope. Usually, the chest x-ray is also normal. Using spiometry to measure forced expiratory volume in 1 second is required to demonstrate airflow obstruction and to make the diagnosis. In a person who has chronic obstructive pulmonary disease, the test shows reduced airflow during a forceful exhalation. As the disease progresses, chest movement diminishes during breathing, and the neck and shoulder muscles participate in the person's labored breathing. Breath sounds become harder to hear through the stethoscope.

If a person develops chronic obstructive pulmonary disease at a young age, alpha1-antitrypsin deficiency is suspected, and the blood level of the protein is measured. It's also measured in family members of a person known to have the deficiency.

Treatment. Because cigarette smoking is the most important cause of COPD, the main treatment is to stop smoking. Stopping smoking when the airflow obstruction is mild or moderate slows the development of disabling. However, stopping smoking at any point in the disease process provides some benefit. The person should also try to avoid exposure to other airborne irritants.

If the person contracts influenza or pneumonia, chronic obstructive pulmonary disease may worsen markedly. Therefore, a person with the disease should receive an influenza vaccination every year and a pneumococcal vaccination once every 6 or so years. The reversible elements of airway obstruction include muscle spasm, inflammation, and increasing secretions. Improvement in any of these elements will generally lessen symptoms. Muscle spasm may be reduced by using bronchodilators, including beta-adrenergic receptor antagonists (such as albuterol in a metered-dose inhaler) and a slowly absorbed form of oral theophylline. Inflammation may be reduced by using corticosteroids, but symptoms respond to corticosteroids in only about 20 percent of patients. There's no reliable therapy for thinning secretions so they can be coughed up more easily. However, avoiding dehydration may prevent thick secretions. A rule of thumb is to drink enough fluids to keep the urine pale except for that passed first in the morning. In severe chronic obstructive pulmonary disease, respiratory therapy may help loosen secretions in the chest.

Flare-ups of chronic obstructive pulmonary disease sometimes result from bacterial infection, which can be treated with antibiotics. A 7- to 10-day course of treatment is often prescribed. Many doctors provide their patients with a supply of an antibiotic and advise them to start taking the drug early in a flare-up. Long-term oxygen therapy prolongs the life of people who have severe chronic obstructive pulmonary disease and severely low oxygen levels in the blood. Although round-the-clock therapy is best, 12 hours of oxygen a day also has some benefit. This therapy reduces the excess of red blood cells caused by low blood oxygen levels, improves the person's mental functioning, and improves the heart failure caused by chronic obstructive pulmonary disease. Oxygen therapy may also improve shortness of breath during exercise. Exercise programs can be carried out in the hospital and at home. These programs can improve the person's independence and quality of life, decrease the frequency and length of hospital stays, and improve the ability to exercise even though lung function doesn't improve. Stationary bicycling, stair climbing, and walking are used to exercise the legs. Weight lifting is used for the arms. Often, oxygen is recommended during exercise. Special techniques are taught for improving function during activities such as cooking, engaging in hobbies, and sexual activity. As with any exercise program, gains in conditioning are quickly lost if the person stops exercising.

For people with a severe alpha1-antitrypsin deficiency, the missing protein can be replaced. The treatment, which requires weekly intravenous infusions of the protein, is expensive. Lung transplantation may be used in selected patients under age 50.

An operation in the early stages of development known as lung volume reduction surgery can be carried out in people with severe emphysema. The procedure is complex, and it requires the person to stop smoking for at least 6 months before surgery and to undergo an intense training program. The operation improves lung function and the ability to exercise in some people, although the duration of the improvement isn't known.

Prognosis. The prognosis for patients with mild airway obstruction is favorable, little worse than the prognosis for smokers without COPD. With moderate and severe airway obstruction, the prognosis becomes progressively worse. About 30 percent of people with the most severe airway obstruction die in 1 year; 95 percent die in 10 years. Death may result from respiratory failure, leakage of air into the pleural space around the lungs (pneumothorax), heart rhythm abnormalities (arrhythmias), or blockage of the arteries leading to the lungs pulmonary embolism). People with chronic obstructive pulmonary disease also have an increased risk of lung cancer. Some people with severe chronic obstructive pulmonary disease may survive for 15 years or more.

Non-human, chimeric, polyclonal (e.g., anti-sera) and/or monoclonal antibodies (Mabs) and fragments (e.g., proteolytic digestion products thereof) are potential therapeutic agents that are being developed in some cases to attempt to treat certain diseases. However, such antibodies that comprise non-human portions elicit an immune response when administered to humans. Such an immune response can result in an immune complex-mediated clearance of the antibodies from the circulation, and make repeated administration unsuitable for therapy, thereby reducing the therapeutic benefit to the patient and limiting the readministration of the Ig derived protein. For example, repeated administration of antibodies comprising non-human portions can lead to serum sickness and/or anaphalaxis. In order to avoid these and other such problems, a number of approaches have been taken to reduce the immunogenicity of such antibodies and portions thereof, including chimerization and “humanization,” as well known in the art. These approaches have produced antibodies having reduced immunogenicity, but with other less disirable properties.

Accordingly, there is a need to provide COPD-related human antibodies or specified portions or variants, nucleic acids, host cells, compositions, and methods of making and using thereof, that overcome one more of these problems, as well as improvements over known human or humanized COPD-related protein antibodies or specified portions or variants thereof.

### SUMMARY OF THE INVENTION

The present invention provides isolated COPD-related human Ig derived proteins (Ig derived proteins), including immunoglobulins, receptor fusion proteins, cleavage products and other specified portions and variants thereof, as well as COPD-related Ig derived protein compositions, encoding or complementary nucleic acids, vectors, host cells, compositions, formulations, devices, transgenic animals, transgenic plants, and methods of making and using thereof, as described and enabled herein, in combination with what is known in the art. Such COPD-related Ig derived proteins act as antagonists to COPD related proteins and thus are useful for treated COPD related pathologies. COPD related proteins include, but are not limited to TNF, IL-6, IL-8, EGF, CD-8 and CD-18. The present invention also provides at least one isolated COPD-related Ig derived protein or specified portion or variant as described herein and/or as known in the art.

The present invention provides, in one aspect, isolated nucleic acid molecules comprising, complementary, or hybridizing to, a polynucleotide encoding specific COPD-related Ig derived proteins or specified portions or variants thereof, comprising at least one specified sequence, domain, portion or variant thereof. The present invention further provides recombinant vectors comprising said isolated COPD-related Ig derived protein nucleic acid molecules, host cells containing such nucleic acids and/or recombinant vectors, as well as methods of making and/or using such Ig derived protein nucleic acids, vectors and/or host cells.

At least one Ig derived protein or specified portion or variant of the invention binds at least one specified epitope specific to at least one COPD-related protein, subunit, fragment, portion or any combination thereof. The at least one epitope can comprise at least one Ig derived protein binding region that comprises at least one portion of said protein, which epitope is preferably comprised of at least one extracellular, soluble, hydrophillic, external or cytoplasmic portion of said protein.

The at least one Ig derived protein or specified portion or variant can optionally comprise at least one specified portion of at least one CDR (e.g., CDR1, CDR2 or CDR3 of the heavy or light chain variable region) and/or at least one framework region. The at least one Ig derived protein or specified portion or variant amino acid sequence can further optionally comprise at least one specified substitution, insertion or deletion.

The present invention also provides at least one composition comprising (a) an isolated COPD-related Ig derived protein or specified portion or variant encoding nucleic acid and/or Ig derived protein as described herein; and (b) a suitable carrier or diluent. The carrier or diluent can optionally be pharmaceutically acceptable, according to known methods. The composition can optionally further comprise at least one further compound, protein or composition.

The present invention also provides at least one method for expressing at least one COPD-related Ig derived protein or specified portion or variant in a host cell, comprising culturing a host cell as described herein and/or as known in the art under conditions wherein at least one COPD-related Ig derived protein or specified portion or variant is expressed in detectable and/or recoverable amounts.

The present invention further provides at least one COPD-related Ig derived protein, specified portion or variant in a method or composition, when administered in a therapeutically effective amount, for modulation, for treating or reducing the symptoms of COPD and related disorders, such as asthma, emphysema, chronic bronchitis or airflow obstruction, as needed in many different conditions, such as but not limited to, prior to, subsequent to, or during a related disease or treatment condition, as known in the art.

The present invention further provides at least one COPD-related Ig derived protein, specified portion or variant in a method or composition, when administered in a therapeutically effective amount, for modulation, for treating or reducing the symptoms of COPD or COPD related disease in a cell, tissue, organ, animal or patient and/or, as needed in many different conditions, such as but not limited to, prior to, subsequent to, or during a related disease or treatment condition, as known in the art and/or as described herein.

The present invention also provides at least one composition, device and/or method of delivery of a therapeutically or prophylactically effective amount of at least one COPD-related Ig derived protein or specified portion or variant, according to the present invention.

### DESCRIPTION OF THE FIGURES

FIG. 1 is a diagram illustrating the current understanding of the pathophysiology of COPD.

### DESCRIPTION OF THE INVENTION

Whereas the present scenario on COPD treatment is decidedly grim, rapid advances in understanding its cellular and molecular pathophysiology give rise to hope that a new generation of drugs will emerge with the potential of slowing disease progression. In particular, activated neutrophils, macrophages and CD8⁺ T cells are associated with COPD, as is mucous gland metaplasia. The immunocompetent cells are recruited and activated by a variety of cytokines (e.g., TNFα, IL-6) and chemokines (e.g., IL-8) that are released in the lung in response to environmental challenge. The architecture of the lung is then destroyed by proteases (e.g., MMP-9, elastase) and reactive oxygen species released from neutrophils and macrophages, as well as by the direct cytotoxic effects of activated CD8⁺ T cells. Likewise, the generation of epidermal growth factor in the lungs of individuals with COPD drives mucous glad metaplasia.

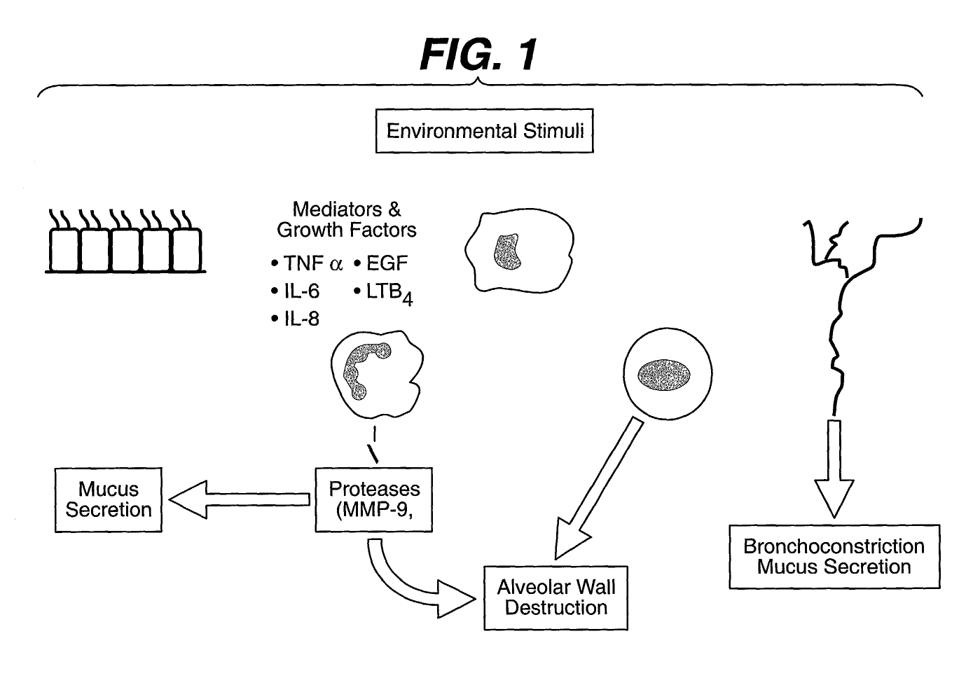

The cells and mediators implicated in the pathophysiology of COPD share a common feature regarding drug development (see FIG. 1) nearly all of them are excellent targets for monoclonal antibodies (mAbs). As a class, mAbs have several advantages over traditional small molecular weight drugs. First, mAbs are highly selective for their molecular targets, thus resulting in predictable biological effects as well as reduced side effects and toxicities. Second, mAbs do not compete with the same drug metabolism and disposition processes that handle small molecules, substantially reducing the risk of drug interactions. Third, the pharmacokinetics of mAbs are predictable and their half lives are long. Finally, a number of characteristics inherent in the discovery and discovery and development of mAbs make R & D cycle times for these agents shorter than those associated with small-molecule drugs.

The present invention provides isolated, recombinant and/or synthetic COPD-related Ig derived proteins or specified portions or variants, as well as compositions and encoding nucleic acid molecules comprising at least one polynucleotide encoding at least one COPD-related Ig derived protein. Such Ig derived proteins or specified portions or variants of the present invention comprise specific full length Ig derived protein sequences, domains, fragments and specified variants thereof, and methods of making and using said nucleic acids and Ig derived proteins or specified portions or variants, including therapeutic compositions, methods and devices.

Abbreviations:

| | |
|---|---|
| MAb | monoclonal antibody |
| COPD | chronic obstructive pulmonary disease |
| IgE | Immunoglobulin E |
| IL | interleukin |
| TNFα | tumor necrosis factor alpha |
| IL-1RA | IL-1 receptor antagonist |
| RANTES | regulated on activation normal T cell |
| | expressed and secreted |
| ICAM-1 | intercellular adherence molecule-1 |
| VLA-4 | very late activating antigen-4 |
| VCAM-1 | vascular cell adhesion molecule-1 |
| MCP | monocyte chemotactic protein |
| MIP | macrophage inflammatory peptide |
| BAL | bronchial alveolar lavage |
| AHR | airway hyper-responsiveness |
| Th | T helper cell |
| CTLA-4 | cytotoxic T-lymphocyte associated antigen 4 |

As used herein, a “Chronic Obstructive Pulmonary Disease Related Ig derived protein,” “COPD-related Ig derived protein,” “COPD-related Ig derived protein portion,” or “COPD-related Ig derived protein fragment” and/or “COPD-related Ig derived protein variant” and the like decreases, blocks, inhibits, abrogates or interferes with COPD-related protein activity, binding or COPD-related protein receptor activity or binding in vitro, in situ and/or preferably in vivo. As presented in FIG. 1, non-limiting examples of COPD related proteins (including receptor proteins), include, but are not limited to tumor necrosis factor alpha (TNF or TNF-alpha) (SEQ ID NOS:1-2); interleukin-6 (IL-6) (SEQ ID NO:3); interleukin-8 (IL-8) (SEQ ID NO:4); epidermal growth factor (EGF) (SEQ ID NO:5); CD-8 (SEQ ID NOS:6-11); and CD-18 (SEQ ID NO:12), CXCR1, CXCR2, MCP-1, C5a Gc globulin, ICAM-1, E-selectin, IL-1, Neutrophil elastase, a cathepsin, an MMP, and the like.

For example, a suitable COPD-related Ig derived protein, specified portion or variant of the present invention can bind at least one COPD-related protein or receptor and includes anti-COPD-related Ig derived proteins, antigen-binding fragments thereof, and specified portions, variants or domains thereof that bind specifically to COPD-related. A suitable COPD-related Ig derived protein, specified portion, or variant can also decrease block, abrogate, interfere, prevent and/or inhibit COPD-related protein RNA, DNA or protein synthesis, COPD-related protein release, COPD-related protein or receptor signaling, membrane COPD-related protein cleavage, COPDprotein related activity, COPD-related protein production and/or synthesis.

Anti-COPD-related Ig derived proteins (also termed COPD-related Ig derived proteins) useful in the methods and compositions of the present invention are characterized by high affinity binding to COPD-related and optionally and preferably having low toxicity. In particular, an Ig derived protein, specified fragment or variant of the invention, where the individual components, such as the variable region, constant region and framework, individually and/or collectively, optionally and preferably possess low immunogenicity, is useful in the present invention. The Ig derived proteins that can be used in the invention are optionally characterized by their ability to treat patients for extended periods with good to excellent alleviation of symptoms and low toxicity. Low immunogenicity and/or high affinity, as well as other suitable properties, may contribute to the therapeutic results achieved. “Low immunogenicity” is defined herein as raising significant HAHA, HACA or HAMA responses in less than about 75%, or preferably less than about 50% of the patients treated and/or raising low titres in the patient treated (less than about 300, preferably less than about 100 measured with a double antigen enzyme immunoassay) (Elliott et al., Lancet 344:1125-1127 (1994), entirely incorporated herein by reference).

Utility

The isolated nucleic acids of the present invention can be used for production of at least one COPD-related Ig derived protein, fragment or specified variant thereof, which can be used to effect in an cell, tissue, organ or animal (including mammals and humans), to modulate, treat, alleviate, help prevent the incidence of, or reduce the symptoms of, at least one COPD-related condition.

Such a method can comprise administering an effective amount of a composition or a pharmaceutical composition comprising at least one anti-COPD-related Ig derived protein or specified portion or variant to a cell, tissue, organ, animal or patient in need of such modulation, treatment, alleviation, prevention, or reduction in symptoms, effects or mechanisms. The effective amount can comprise an amount of about 0.001 to 500 mg/kg per single or multiple administration, or to achieve a serum concentration of 0.01-5000 μg/ml serum concentration per single or multiple adminstration, or any effective range or value therein, as done and determined using known methods, as described herein or known in the relevant arts.

Citations

All publications or patents cited herein are entirely incorporated herein by reference as they show the state of the art at the time of the present invention and/or to provide description and enablement of the present invention. Publications refer to any scientific or patent publications, or any other information available in any media format, including all recorded, electronic or printed formats. The following references are entirely incorporated herein by reference: Ausubel, et al., ed., Current Protocols in Molecular Biology, John Wiley & Sons, Inc., NY, N.Y. (1987-2001); Sambrook, et al., Molecular Cloning: A Laboratory Manual, 2^{nd} Edition, Cold Spring Harbor, N.Y. (1989); Harlow and Lane, Ig derived proteins, a Laboratory Manual, Cold Spring Harbor, N.Y. (1989); Colligan, et al., eds., Current Protocols in Immunology, John Wiley & Sons, Inc., New York (1994-2001); Colligan et al., Current Protocols in Protein Science, John Wiley & Sons, NY, N.Y., (1997-2001).

Given the well-described presence of neutrophils and T lymphocytes in the lungs of individuals with COPD, chemokines and cytokine that support the infiltration and activation of these cell types become obvious targets. Characterizing COPD as a chronic inflammatory disease naturally makes Interleukin-1 (IL-1) and tumor necrosis factor alpha (TNF-α candidates as therapeutic targets. Both of these cytokines are strongly associated with inflammatory processes. Biopharmaceuticals interfering with the action of these cytokines currently exist and are being evaluated in the clinic, although not for COPD. The naturally occurring antagonist to IL-1 (IL-IRA) has shown utility in rheumatoid arthritis (15). The use of a soluble receptor-fusion protein (Enbrel®) in rheumatoid arthritis has proven positive (15). Furthermore, the recently published data with an anti-TNF mAb, infliximab (Remicade®), in psoriasis, Crohn's disease and rheumatoid arthritis bodes well for the efficacy of biopharmaceuticals for chronic inflammatory diseases (16) (17).

Downstream in the inflammatory cascade, interleukin-6 (IL-6) is generated as a consequence of the inflammatory process in COPD, and its over-expression in transgenic mice produces an emphysematous phenotype (18). Interleukin-8 (IL-8) mediates neutrophil chemotaxis through its interaction with CXCR-2 and degranulates neutrophils through an interaction with CXCR-1 (19). Interleukin-8 is a particularly attractive candidate when one takes into account the fact that activated T-cell chemotaxis is dependent upon IL-8 and CXCR-2 (S. Sarau, personal communication). Thus, a mAb to IL-8 would be a likely candidate for therapy of COPD. The humanized mAb from Abgenix has not been evaluated in this patient population but early clinical studies suggest activity against psoriasis (20), a Th-1-associated skin disease with a characteristic neutrophil infiltration.

Other chemokines such as RANTES (Regulated on Activation, Normal T-cell Expressed and Secreted), which has been associated with monocyte and memory T-cell chemotaxis, is a potential mediator of chronic pulmonary inflammation (21). Monocyte chemotactic protein-1 may also be a therapeutic target. Proteins involved with the adherence, rolling and diapedesis of inflammatory cells are likewise excellent targets. These include proteins such as intercellular adherence molecule-1 (ICAM-1), very late activating antigen-4 (VLA-4), and the beta integrin CD18. In a similar fashion, targeting of CD8 on T-lymphocytes will likely reduce lung damage and inflammatory cell infiltration. In that regard, anti-ICAM-1 antibody blocks pulmonary inflammation in viral bronchiolitis in rats, and anti VLA-4 inhibited increases in lung resistance and inflammation in ovalbumin-sensitized animals (22) (23).

Further down the cascade, the products of activated inflammatory cells (e.g. proteases) are also likely targets. In the latter case proteins such as matrix metalloproteinases and cathepsins may play and important role in tissue destruction (24). Ultimately, remodeling and emphysema may also involve Interleukin-13 (IL-13). In that regard, inducible targeting of IL-13 in adult mice caused MMP- and cathepsin-dependent emphysema (25).

Thus, a number of potential molecular targets exist for mAb-based therapy of COPD, along with a supporting scientific rationale (See Table I). As detailed in Table II, several monoclonal antibodies and/or biological reagents exist to test the proof of principle either in the clinic or in animal models. To our knowledge, none of these monoclonal antibodies have been evaluated clinically in patients with COPD.

Monoclonal Antibodies in the Treatment of Asthma

There is strong evidence that CD4+ T cells, especially the Th2 subtype, play a crucial role in the pathogenesis of allergic asthma (26). Therefore, therapeutic strategies targeting CD4⁺ T cells, T cell co-stimulatory molecules and Th2 cytokines may offer effective approaches to control asthma (Table III & IV). A chimeric anti-CD4 monoclonal antibody, keliximab, was evaluated in severe corticosteroid-dependent asthma patients. Significant improvement in lung functions and overall symptom scores were demonstrated, which was accompanied by a marked reduction in CD4⁺ T cell counts (27). Furthermore, a non-depleting anti-CD4 antibody induced anergy to allergen specific CD4⁺ T cells and prevented the development of asthma in a mouse (28).

CD28 on T cells and B7 on APCs are particularly important for T cell activation (29). Interruption of their interaction normally induces T cell anergy, thereby inhibiting T cell-mediated disease. In animal asthma models, both CTLA-4-Ig fusion protein (binding to B7) and anti-B7-2 antibody suppressed allergen induced AHR, IgE production and eosinophil recruitment (30) (31). Ex vivo studies showed that both reagents inhibited allergen induced proliferation and cytokine production by peripheral blood mononuclear cells from atopic subjects (32). Furthermore, CTLA-4-Ig blocked allergen induced cytokine production in bronchial explants from atopic asthmatics (33).

These results suggest the potential use of CTLA-4-Ig or anti-B7 antibodies as treatment for allergic asthma.

Many efforts are focused on blocking Th2-derived cytokines. Anti-IL-4 monoclonal antibodies inhibited IgE production, eosinophil influx and AHR in rodent models of asthma (34). An anti-IL-4 antibody, SB 240683, and a soluble IL-4 receptor have been developed to treat asthma. In a Phase I/II trial, a single dose of sIL-4R significantly improved pulmonary function and stabilized symptom scores in moderate asthmatic patients, even with abrupt withdrawal of corticosteroids (35). Subsequent studies, however, were disappointing and the development of this agent was recently terminated (36). Two humanized anti-IL-5 antibodies, SCH55700 and SB 240563, have also been evaluated in clinical trials. Despite the ability of these agents to reduce substantially eosinophils in venous blood and sputum, neither antibody had effect on late phase airway responsiveness (3) (37). These somewhat unexpected results raise serious questions about the previously well-accepted role that eosinophils and IL-5 play in asthma pathogenesis. Two additional Th2-derived cytokines, IL-9 and IL-13, are up regulated in the lung of asthmatics and have genetic polymorphisms linked to asthma (38) (39). An anti-IL-9 antibody and an IL-13R-Fc fusion protein suppressed both AHR and mucus production in animal models, which suggests that IL-9 and IL-13 play roles in the pathogenesis of asthma (40) (41) (42) (43).

IgE is an important trigger of allergic asthma, and asthma symptoms strongly correlate with elevated serum IgE levels and skin test reactivity (44). Cross-linking of IgE bound to mast cells or basophils by allergen causes de-granulation of these cells, resulting in the archetypal immediate asthmatic response (44). Recombinant humanized anti-IgE monoclonal antibodies (rhuMAb-E25 and CGP 56901) have been developed, which block the binding of IgE to its high affinity receptor, thereby preventing the release of mediators (45). In several clinical trials, rhuMAb-E25 dramatically reduced serum levels of free IgE, attenuated both early and late phase responses to inhaled allergen, and significantly reduced asthma symptom (46).

Another strategy to mitigate the role of IgE in asthma is to block its low affinity receptor, CD23, or the processing of CD23. In animal models, anti-CD23 antibodies significantly inhibited allergen-induced pulmonary inflammation and airway responsiveness (47). Moreover, the use of an anti-CD23 mAb to inhibit the proteolytic cleavage of CD23 abolished IL-4-stimulated IgE synthesis in human B cells adoptively transferred into SCID (48). This suggests that CD23 processing is an obligatory step in IL-4-induced IgE synthesis. The role of CD23 in asthma may be complex, however, as enhanced airway hyper-reactivity was observed in CD23 deficient mice (49). Several anti-CD23 antibodies are in development.

Chemokines and their receptors have been implicated in the pathophysiology of airway inflammation associated with asthma (50) (51). Particular attention has been paid to eotaxin, eotaxin-2, MCPs, MIP-1α and RANTES (52). In animal models, anti-MCP-1 and anti-MCP-3 antibodies inhibited allergen-induced monocyte and eosinophil infiltration (53) (54). But due to the highly complex redundancy of biological activities among chemokines, especially the ability of several chemokines to signal through a common receptor, neutralizing an individual chemokine may not have a therapeutically meaningful impact on the pathophysiology of asthma. Indeed, eotaxin-deficient mice only show partial reduction in allergen induced pulmonary eosinophilia (55). Theoretically, targeting chemokine receptors rather than the chemokines themselves could circumvent the issue of several chemokines acting through the same receptor. To date, however, producing blocking monoclonal antibodies against G-protein-coupled receptors has been notoriously difficult.

Adhesion molecules serve as the final common pathway for mediating cellular recruitment in the lung (56). The VLA-4/VCAM-1 interaction is particularly important in asthma. Anti-VLA-4 antibodies and a VCAM-Ig fusion protein reduced airway inflammation and airway responsiveness in a number of animal models (23). A therapeutic monoclonal antibody, natalizumab (Antegren®), is currently under development for multiple sclerosis. Antibodies to ICAM-1 were also active in animal models of asthma, and the airway responsiveness and cellular infiltration were substantially attenuated in ICAM-1^{-/-} mice (57) (22).

The pluripotential pro-inflammatory cytokines, TNF and IL-1, may also play important roles in the pathophysiology of asthma. The expression of both TNF and IL-1 is increased in the lungs of asthmatic patients (58) (59). An anti-IL-1 antibody reduced pulmonary resistance, decreased the percentage of hypodense eosinophils in BAL, and inhibited IgE synthesis and pro-inflammatory cytokine production (60) (61). As in the case of COPD, a strong rationale exists for the potential utility of the anti-TNFα mAb, infliximab (Remicade®), in asthma.

Conclusions

The use of biological therapeutics in chronic inflammatory disease has taken great strides forward. The ability to utilize one or more monoclonal antibodies directed toward offending mediators or cells to treat these diseases allows one to tailor highly specific therapeutics to patient populations with reduced concern about non-mechanism based toxicities. With careful study of the mechanisms of disease pathogenesis and judiciously selected clinical proof-of-concept studies, we are standing on the brink of an era in which biopharmaceuticals could have a major impact on the progression and underlying pathogenesis of pulmonary diseases.

TABLE I
Antibody targets for COPD therapeutics

| Category | Target | Biological Activities Involved in the Pathogenesis | Ref. |
|---|---|---|---|
| Chemokines & | IL-8 | Stimulates neutrophil chemotaxis and migration | (62) |
| Chemokine | | Induces neutrophil activation and degranulation | |
| Receptors | | Induces tissue neutrophilia | |
| | CXCR1 | Mainly expressed on neutrophils, but also on | (19) |
| | | T cells and monocytes | |
| | | Mediates IL-8 induced neutrophil chemotaxis and | |
| | | Ca⁺⁺ flux | |
| | CXCR2 | Expressed on neutrophils, T cells, B cells and | (19) |
| | | monocytes | |
| | | Mediates IL-8, Gro- and NAP-2 induced neutrophil | |
| | | chemotaxis | |
| | MCP-1 | See Table III | |
| | C5a | Levels increased in COPD lungs | (63) |
| | Gc-globulin | Mediates recruitment of neutrophils and monocytes | |
| Adhesion | ICAM-1 | Level increased in bronchial biopsies of patients | (62) |
| Molecules | | with COPD | |
| | | Mediates migration of T cells and monocytes into | |
| | | inflamed lung through binding to VAL-4 | |
| | CD18 | Mediates macrophage and neutrophil attachment to | (64) |
| | | endothelial cells | |
| | E-selectin | Expression increases in COPD lung | (65) |
| | | Mediates neutrophil recruitment | (66) |
| Pro- | TNF | See Table III | |
| inflammatory | IL-1 | See Table III | |
| Cytokines | IL-6 | Level increases in COPD lung. | (18) |
| | | Over-expression induces emphysema and airway | |
| | | remodeling. | |
| Proteases | Neutrophil | Level increases in COPD lungs | (67) |
| | elastase | Digests elastin and reduces elastic quality of the | |
| | | lung. | |
| | | Induces IL-8 release from epithelial cells | |
| | | Stimulates mucous secretion | |
| | Cathepsins | Shows similar elastolytic activity as elastase | (62) |
| | MMPs | A group of over 20 closely related endopeptidases | (62) |
| | | Degrades components of the extra-cellular matrix of | |
| | | lung parenchyma including elastin, collagen, | |
| | | proteoglycans, laminin and fibronectin | |
| | | MMP-1 and MMP-9 are up regulated in BAL of | |
| | | emphysema patients | |

TABLE II
Monoclonal Antibodies in Clinical Development with
Potential Utility in COPD

| Agent | Company | Mechanism | Indication |
|---|---|---|---|
| Cytolin | CytoDyn | Anti-CD8 | Adjunct to |
| | | | antiviral |
| | | | HIV |
| LDP-01 | Millennium | Anti-CD18 | Neuroprotection |
| | INSERM | Anti-IL-6 | Cancer |
| ABX-IL-8 | Abgenix | Anti-IL-8 | Psoriasis |
| cetuzimab | ImClone | Anti-EGF | Cancer |
| | | Receptor | |
| infliximab | Centocor | Anti-TNF | RA, Crohn's |

TABLE III
Antibody targets for asthma therapeutics

| Category | Target | Biological Activities Involved in Pathogenesis | Ref. |
|---|---|---|---|
| IgE & | IgE | Serum IgE level is higher in asthmatics | (44) |
| IgE receptor | | Cross-linking membrane bound IgE causes de- | |
| | | granulation of mast cells and basophils | |
| | | Mediators released from mast cells and basophils | |
| | | induce vascular permeability, mucus production and | |
| | | airway hyper-responsiveness | |
| | CD23 | Low affinity IgE receptor | (47) |
| | | Facilitates allergen presentation to T cells | |
| | | Activates macrophages and induces cytokine | |
| | | secretion through the binding to CD11b and CD11c | |
| Th2 | IL-4 | Induces Th2 cell and inhibits Th1 cell differentiation | (34) |
| Cytokine | | Induces B cell activation and proliferation | |
| | | Induces IgE isotype switching and increases IgE | |
| | | production | |
| | | Increases VCAM-1 expression on endothelial cells | |
| | | Increases eotaxin production by fibroblasts | |
| | | Induces mucus production | |
| | IL-5 | Induces growth, differentiation, proliferation of | (34) |
| | | bone marrow eosinophils | |
| | | Prolongs eosinophil survival. | |
| | | Induces eosinophil tissue recruitment and | |
| | | eosinophilic inflammation | |
| | IL-9 | Induces mast cell proliferation and activation in | (39) |
| | | synergy with IL-3 and IL-4 | |
| | | Contributes to mastocytosis and mucosal mast cell | |
| | | hyperplasia. | |
| | | Potentiates IL-4 induces IgE and IgG production. | |
| | | Over expressed IL-9 in the lung Induces MCP-1, 3, | |
| | | 5 and eotaxin production, eosinophilia and AHR. | |
| | IL-13 | Promotes growth and activation of B cells | (38) |
| | | Induces immunoglobulin class switching to IgE and | |
| | | IgG4. | |
| | | Induces VCAM-1 expression on endothelial cells | |
| | | Induces Eotaxin expression by pulmonary epithelial | |
| | | cells | |
| | | Over expressed IL-13 in the lung induces | |
| | | eosinophilia, mucus production, AHR and fibrosis | |
| T Cell | CD4 | Mediates CD4 T cell activation and signal | (68) |
| Stimulatory | | transduction. | |
| Molecule | | CD4+ Th2 cells regulate allergic asthma through | |
| | | cytokines. | |
| | | Depletion of CD4 T cells blocks asthma | |
| | | development in animal models. | |
| | | Adoptively transferred CD4 Th2 cells induces | |
| | | asthma in rodents | |
| | CD28- | Mediates co-stimulatory signals resulting in | (29) |
| | CD86 | complete T cell activation. | |
| Chemokines & | Eotaxin | Up regulated in the lung of asthmatic. | (69) |
| Chemokine | | Induces eosinophil chemotaxis and activation. | |
| Receptors | | Induces eosinophilia in synergy with IL-5. | |
| | CCR3 | Mainly expressed on eosinophils, mast cells, | (70) |
| | | basophils and Th2 cells | |
| | | Mediates chemotaxis and C⁺⁺ flux induced by | |
| | | eotaxin and eotaxin-2 as well as RANTES and | |
| | | MCP-3 | |
| | MCPs | CC chemokines that are up regulated in the lung of | (50) |
| | | asthmatic | |
| | | Induces recruitment of T cells, monocytes and | |
| | | eosinophils | |
| | | MCP-1 enhances Th2 cell development | |
| | RANTES | CC chemokines that are increased in the lung of | (50) |
| | | asthmatic | |
| | | Induce recruitment of T cells, monocytes and | |
| | | eosinophils | |
| Adhesion | VLA-4 | Mediates migration of T cells, eosinophils and | (23) |
| Molecules | | monocytes into inflamed tissue through binding to | |
| | | VCAM-1 and fibronectin | |
| | | T cell co-stimulatory molecule | |
| | VACM-1 | Mediates migration of T cells, eosinophils and | (56) |
| | | monocytes into inflamed lung through binding to | |
| | | VAL-4 | |
| Pro- | TNFα | Production is increased in asthmatic airways | (58) |
| inflammatory | | Activates monocytes and neutrophils | |
| Cytokines | | Induces the production of other pro-inflammation | |
| | | cytokines e.g. IL-1 and IL-6 and chemokines by | |
| | | monocytes, endothelial and epithelial cells | |
| | | Up regulates adhesion molecule expression on | |
| | | endothelial cells | |
| | | Increases vascular permeability | |
| | IL-1 α/β | Production is increased in asthmatic airways | |
| | | Supports T cell activation | |
| | | Enhances basophil histamine release | |
| | | Enhances release of prostaglandin E2 and | |
| | | leukotriene B4 | |
| | | Induces production of TNF, IL-6 and GM-CSF | |
| | | Induces adhesion molecule expression on | |
| | | endothelial cells | |

TABLE IV
Monoclonal Antibodies in Clinical Development with
Potential Utility in Asthma

| Agent | Company | Mechanism | Indication |
|---|---|---|---|
| rhuMAb- | Genentech | Anti-IgE | Asthma; |
| E25 | | | allergy; |
| | | | rhinitis |
| CGP | Tanox | Anti-IgE | Asthma; |
| 56901 | | | allergy; |
| | | | rhinitis |
| mAb, | IDEC | Anti-CD23 | Asthma; |
| CD23 | | | allergy; |
| | | | rhinitis |
| clenolix | IDEC | Anti-CD4 | RA; |
| imab | | | Psoriasis; |
| | | | asthma |
| SB | GlaxoSmith | Anti-IL-4 | Asthma |
| 240683 | Kline | | |
| Nuvance | Immunex | Soluble IL- | ADIS; |
| | | 4 receptor | asthma; |
| | | | allergy; |
| | | | transplant |
| | | | rejection |
| SB | GlaxoSmith | Anti-IL-5 | Asthma |
| 240563 | Kline | | |
| SCH | Schering- | Anti-IL-5 | Asthma; |
| 55700 | Plough | | allergy |

Ig Derived Proteins of the Present Invention

The term “Ig derived protein “is intended to encompass Ig derived proteins, digestion fragments, specified portions and variants thereof, including Ig derived protein mimetics or comprising portions of Ig derived proteins that mimic the structure and/or function of an anitbody or specified fragment or portion thereof, including single chain Ig derived proteins and fragments thereof, and is also is intended to encompass proteins that contain mimetics to therapeutic proteins, antibodies, and digestion fragments, specified portions and variants thereof, wherein the protein comprises at least one functional COPD related protein ligand binding region (LBR) that optionally replaces at least one complementarity determing region (CDR) of the antibody from which the Ig-derived protein, portion or variant is derived. Such COPD related IgG derived proteins, specified portions or variants include those that mimic the structure and/or function of at least one COPD related protein antagonist, such as a COPD related protein antibody or receptor or ligand protein, or fragment or analog. Functional fragments include antigen-binding fragments that bind to human COPD-related proteins. For example, Ig derived protein fragments capable of binding to human COPD-related or portions thereof, including, but not limited to Fab (e.g., by papain digestion), Fab′ (e.g., by pepsin digestion and partial reduction) and F(ab′)₂ (e.g., by pepsin digestion), facb (e.g., by plasmin digestion), pFc′ (e.g., by pepsin or plasmin digestion), Fd (e.g., by pepsin digestion, partial reduction and reaggregation), Fv or scFv (e.g., by molecular biology techniques) fragments, are encompassed by the invention (see, e.g., Colligan, Immunology, supra).

Such fragments can be produced by enzymatic cleavage, synthetic or recombinant techniques, as known in the art and/or as described herein. Ig derived proteins can also be produced in a variety of truncated forms using Ig derived protein genes in which one or more stop codons have been introduced upstream of the natural stop site. For example, a chimeric gene encoding a F(ab′)₂ heavy chain portion can be designed to include DNA sequences encoding the CH₁ domain and/or hinge region of the heavy chain. The various portions of Ig derived proteins can be joined together chemically by conventional techniques, or can be prepared as a contiguous protein using genetic engineering techniques. For example, a nucleic acid encoding the variable and constant regions of a human Ig derived protein chain can be expressed to produce a contiguous protein. See, e.g., Colligan, Immunology, supra, sections 2.8 and 2.10, for fragmentation and Ladner et al., U.S. Pat. No. 4,946,778 and Bird, R. E. et al., Science, 242: 423-426 (1988), regarding single chain Ig derived proteins, each of which publications are entirely incorporated herein by reference.

As used herein, the term “human Ig derived protein” refers to an Ig derived protein in which substantially every part of the protein (e.g., CDR, LBR, framework, C_{L}, C_{H}domains (e.g., C_{H}1, C_{H}2, C_{H}3), hinge, (V_{L}, V_{H})) is substantially non-immunogenic, with only minor sequence changes or variations. Such changes or variations optionally and preferably retain or reduce the immunogenicity in humans relative to non-modified human Ig derived proteins. Thus, a human Ig derived protein is distinct from a chimeric or humanized Ig. It is pointed out that a human Ig derived protein can be produced by a non-human animal or prokaryotic or eukaryotic cell that is capable of expressing functionally rearranged human immunoglobulin (e.g., heavy chain and/or light chain) genes. Further, when a human Ig derived protein is a single chain Ig derived protein, it can comprise a linker peptide that is not found in native human Ig derived proteins. For example, an Fv can comprise a linker peptide, such as two to about eight glycine or other amino acid residues, which connects the variable region of the heavy chain and the variable region of the light chain. Such linker peptides are considered to be of human origin. COPD related Ig derived proteins that comprise at least one COPD related protein ligand or receptor thereof can be designed against an appropriate ligand, such as isolated and/or COPD related protein, or a portion thereof (including synthetic molecules, such as synthetic peptides). Preparation of such COPD related Ig derived proteins are performed using known techniques to identify and characterize ligand binding regions or sequences of at least one COPD related protein or portion thereof.

Human Ig derived proteins that are specific for human COPD-related proteins or fragments thereof can be raised against an appropriate immunogenic antigen, such as isolated and/or COPD-related protein or a portion thereof (including synthetic molecules, such as synthetic peptides). Preparation of immunogenic antigens, and monoclonal Ig derived protein production can be performed using any suitable technique. A variety of methods have been described (see e.g., Kohler et al., Nature, 256: 495-497 (1975) and Eur. J. Immunol. 6: 511-519 (1976); Milstein et al., Nature 266: 550-552 (1977); Koprowski et al., U.S. Pat. No. 4,172,124; Harlow, E. and D. Lane, 1988, Ig derived proteins: A Laboratory Manual, (Cold Spring Harbor Laboratory: Cold Spring Harbor, N.Y.); Current Protocols In Molecular Biology, Vol. 2 (e.g., Supplement 27, Summer '94), Ausubel, F. M. et al., Eds., (John Wiley & Sons: New York, N.Y.), Chapter 11, (1991-2001)). Generally, a hybridoma is produced by fusing a suitable immortal cell line (e.g., a myeloma cell line such as, but not limited to, Sp2/0, Sp2/0-AG14, NSO, NS1, NS2, AE-1, L.5,>243, P3X63Ag8.653, Sp2 SA3, Sp2 MAI, Sp2 SS1, Sp2 SA5, U937, MLA 144, ACT IV, MOLT4, DA-1, JURKAT, WEHI, K-562, COS, RAJI, NIH 3T3, HL-60, MLA 144, NAMAIWA, NEURO 2A, or the like, or heteromylomas, fusion products thereof, or any cell or fusion cell derived therefrom, or any other suitable cell line as known in the art, see, e.g., www.atcc.org, www.lifetech.com., and the like) with Ig derived protein producing cells, such as, but not limited to, isolated or cloned spleen cells, or any other cells expressing heavy or light chain constant or variable or framework or CDR sequences, either as endogenous or heterologous nucleic acid, as recombinant or endogenous, viral, bacterial, algal, prokaryotic, amphibian, insect, reptilian, fish, mammalian, rodent, equine, ovine, goat, sheep, primate, eukaryotic, genomic DNA, cDNA, rDNA, mitochondrial DNA or RNA, chloroplast DNA or RNA, hnRNA, mRNA, tRNA, single, double or triple stranded, hybridized, and the like or any combination thereof. See, e.g., Ausubel, supra, and Colligan, Immunology, supra, chapter 2, entirely incorporated herein by reference.

Ig derived protein producing cells can be obtained from the peripheral blood or, preferably the spleen or lymph nodes, of humans or other suitable animals that have been immunized with the antigen of interest. Any other suitable host cell can also be used for expressing heterologous or endogenous nucleic acid encoding an Ig derived protein, specified fragment or variant thereof, of the present invention. The fused cells (hybridomas) or recombinant cells can be isolated using selective culture conditions or other suitable known methods, and cloned by limiting dilution or cell sorting, or other known methods. Cells which produce Ig derived proteins with the desired specificity can be selected by a suitable assay (e.g., ELISA).

Other suitable methods of producing or isolating Ig derived proteins of the requisite specificity can be used, including, but not limited to, methods that select recombinant Ig derived protein from a peptide or protein library (e.g., but not limited to, a bacteriophage or ribosome display library; e.g., as available from Cambridge Ig derived protein Technologies, Cambridgeshire, UK; MorphoSys, Martinsreid/Planegg, DE; Biovation, Aberdeen, Scotland, UK; BioInvent, Lund, Sweden; Dyax Corp., Enzon, Affymax/Biosite; Xoma, Berkeley, Calif.; Ixsys; U.S. Pat. Nos. EP 368,684, PCT/GB91/01134; PCT/GB92/01755; PCT/GB92/002240; PCT/GB92/00883; PCT/GB93/00605; U.S. Ser. No. 08/350,260(5/12/94); PCT/GB94/01422; PCT/GB94/02662; PCT/GB97/01835; (CAT/MRC); WO90/14443; WO90/14424; WO90/14430; PCT/US94/1234; WO92/18619; WO96/07754; (Scripps); EP 614 989 (MorphoSys); WO95/16027 (BioInvent); WO88/06630; WO90/3809 (Dyax); U.S. Pat. No. 4,704,692 (Enzon); PCT/US91/02989 (Affymax); WO89/06283; EP 371 998; EP 550 400; (Xoma); EP 229 046; PCT/US91/07149 (Ixsys); or stochastically generated peptides or proteins—U.S. Pat. Nos. 5,723,323, 5,763,192, 5,814,476, 5,8174,83, 5,824,514, 5,976,862, WO 86/05803, EP 590 689 (Ixsys, now Applied Molecular Evolution (AME), each entirely incorporated herein by reference) or that rely upon immunization of transgenic animals (e.g., SCID mice, Nguyen et al., Microbiol. Immunol. 41:901-907 (1997); Sandhu et al., Crit. Rev. Biotechnol. 16:95-118 (1996); Eren et al., Immunol. 93:154-161 (1998), each entirely incorporated by reference as well as related patents and application) that are capable of producing a repertoire of human Ig derived proteins, as known in the art and/or as described herein. Additional techniques, include, but are not limited to, ribosome display (Hanes et al., Proc. Natl. Acad. Sci. USA, 94:4937-4942 (May 1997); Hanes et al., Proc. Natl. Acad. Sci. USA, 95:14130-14135 (November 1998)); single cell Ig derived protein producing technologies (e.g., selected lymphocyte Ig derived protein method (“SLAM”) (U.S. Pat. No. 5,627,052, Wen et al., J. Immunol. 17:887-892 (1987); Babcook et al., Proc. Natl. Acad. Sci. USA 93:7843-7848 (1996)); gel microdroplet and flow cytometry (Powell et al., Biotechnol. 8:333-337 (1990); One Cell Systems, Cambridge, Mass.; Gray et al., J. Imm. Meth. 182:155-163 (1995); Kenny et al., Bio/Technol. 13:787-790 (1995)); B-cell selection (Steenbakkers et al., Molec. Biol. Reports 19:125-134 (1994); Jonak et al., Progress Biotech, Vol. 5, In Vitro Immunization in Hybridoma Technology, Borrebaeck, ed., Elsevier Science Publishers B.V., Amsterdam, Netherlands (1988)).

Methods for humanizing non-human Ig derived proteins can also be used and are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers (Jones et al., Nature 321:522 (1986); Riechmann et al., Nature 332:323 (1988); Verhoeyen et al., Science 239:1534 (1988)), by substituting rodent CDRs or CDR sequences for the corresponding sequences of a human antibody. Accordingly, such “humanized” Ig derived proteins are chimeric Ig derived proteins (Cabilly et al., supra), wherein substantially less than an intact human variable domain has been substituted by the corresponding sequence from a non-human species. In practice, humanized Ig derived proteins are typically human Ig derived proteins in which some CDR residues and possibly some FR residues are substituted by residues from analogous sites in rodent Ig derived proteins.

The choice of human variable domains, both light and heavy, to be used in making the humanized Ig derived proteins can be used to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol. 151: 2296 (1993); Chothia and Lesk, J. Mol. Biol. 196:901 (1987)). Another method uses a particular framework derived from the consensus sequence of all human Ig derived proteins of a particular subgroup of light or heavy chains. The same framework can be used for several different humanized Ig derived proteins (Carter et al., Proc. Natl. Acad. Sci. U.S.A. 89:4285 (1992); Presta et al., J. Immunol. 151:2623 (1993)).

Ig derived proteins can also optionally be humanized with retention of high affinity for the antigen and other favorable biological properties. To achieve this goal, according to a preferred method, humanized Ig derived proteins are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the consensus and import sequences so that the desired antibody characteristic, such as increased affinity for the target antigen(s), is achieved. In general, the CDR residues are directly and most substantially involved in influencing antigen binding.

Human monoclonal Ig derived proteins can be made by the hybridoma method. Human myeloma and mouse-human heteromyeloma cell lines for the production of human monoclonal Ig derived proteins have been described, for example, by Kozbor, J. Immunol. 133:3001 (1984); Brodeur et al., Monoclonal Antibody Production Techniques and Applications, pp. 51-63 (Marcel Dekker, Inc., New York, 1987); and Boerner et al., J. Immunol. 147:86 (1991).

Alternatively, phage display technology (McCafferty et al., Nature 348:552 (1990)) and as presented above can be used to produce human Ig derived proteins and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson et al., Current Opinion in Structural Biology 3:564 (1993). Several sources of V-gene segments can be used for phage display. Clackson et al., Nature 352:624 (1991) isolated a diverse array of anti-oxazolone Ig derived proteins from a small random combinatorial library of V genes derived from the spleens of immunized mice. A repertoire of V genes from unimmunized human donors can be constructed and Ig derived proteins to a diverse array of antigens (including self-antigens) can be isolated essentially following the techniques described by Marks et al., J. Mol. Biol. 222:581 (1991), or Griffith et al., EMBO J. 12:725 (1993).

In a natural immune response, antibody genes accumulate mutations at a high rate (somatic hypermutation). Some of the changes introduced will confer higher affinity, and B cells displaying high-affinity surface immunoglobulin are preferentially replicated and differentiated during subsequent antigen challenge. This natural process can be mimicked by employing the technique known as “chain shuffling” (Marks et al., Bio/Technol. 10:779 (1992)). In this method, the affinity of “primary” human Ig derived proteins obtained by phage display can be improved by sequentially replacing the heavy and light chain V region genes with repertoires of naturally occurring variants (repertoires) of V domain genes obtained from unimmunized donors. This technique allows the production of Ig derived proteins and antibody fragments with affinities in the nM range. A strategy for making very large phage antibody repertoires has been described by Waterhouse et al., Nucl. Acids Res. 21:2265 (1993). Gene shuffling can also be used to derive human Ig derived proteins from rodent Ig derived proteins, where the human antibody has similar affinities and specificities to the starting rodent antibody. According to this method, which is also referred to as “epitope imprinting”, the heavy or light chain V domain gene of rodent Ig derived proteins obtained by phage display technique is replaced with a repertoire of human V domain genes, creating rodent-human chimeras. Selection with antigen results in isolation of human variable capable of restoring a functional antigen-binding site, i.e. the epitope governs (imprints) the choice of partner. When the process is repeated in order to replace the remaining rodent V domain, a human antibody is obtained (see PCT WO 93/06213, published Apr. 1, 1993). Unlike traditional humanization of rodent Ig derived proteins by CDR grafting, this technique provides completely human Ig derived proteins, which have no framework or CDR residues of rodent origin.

Bispecific Ig derived proteins can also be used that are monoclonal, preferably human or humanized, Ig derived proteins that have binding specificities for at least two different antigens. In the present case, one of the binding specificities is for at least one COPD-related protein, the other one is for any other antigen. For example, bispecific Ig derived proteins specifically binding a COPD-related protein and at least one neurotrophic factor, or two different types of COPD-related polypeptides are within the scope of the present invention.

Methods for making bispecific Ig derived proteins are known in the art. Traditionally, the recombinant production of bispecific Ig derived proteins is based on the co-expression of two immunoglobulin heavy chain-light chain pairs, where the two heavy chains have different specificities (Milstein and Cuello, Nature 305:537 (1983)). Because of the random assortment of immunoglobulin heavy and light chains, these hybridomas (quadromas) produce a potential mixture of 10 different antibody molecules, of which only one has the correct bispecific structure. The purification of the correct molecule, which is usually done by affinity chromatography steps, is rather cumbersome, and the product yields are low. Similar procedures are disclosed in WO 93/08829 published May 13, 1993, and in Traunecker et al., EMBO J. 10:3655 (1991), entirely incorporated herein by referece.

According to a different and more preferred approach, antibody-variable domains with the desired binding specificities (antibody-antigen combining sites) are fused to immunoglobulin constant-domain sequences. The fusion preferably is with an immunoglobulin heavy-chain constant domain, comprising at least part of the hinge, the second heavy chain constant region (C.sub.H 2), and the third heavy chain constant region (C.sub.H 3). It is preferred to have the first heavy-chain constant region (C.sub.H 1), containing the site necessary for light-chain binding, present in at least one of the fusions. DNAs encoding the immunoglobulin heavy chain fusions and, if desired, the immunoglobulin light chain, are inserted into separate expression vectors, and are co-transfected into a suitable host organism. This provides for great flexibility in adjusting the mutual proportions of the three polypeptide fragments in embodiments when unequal ratios of the three polypeptide chains used in the construction provide the optimum yields. It is, however, possible to insert the coding sequences for two or all three polypeptide chains in one expression vector when the production of at least two polypeptide chains in equal ratios results in high yields or when the ratios are of no particular significance. In a preferred embodiment of this approach, the bispecific Ig derived proteins are composed of a hybrid immunoglobulin heavy chain with a first binding specificity in one arm, and a hybrid immunoglobulin heavy chain-light chain pair (providing a second binding specificity) in the other arm. This asymmetric structure facilitates the separation of the desired bispecific compound from unwanted immunoglobulin chain combinations, as the presence of an immunoglobulin light chain in only one half of the bispecific molecule provides for a facile way of separation. For further details of generating bispecific Ig derived proteins, see, for example, Suresh et al., Methods in Enzymology 121:210 (1986).

Heteroconjugate Ig derived proteins are also within the scope of the present invention. Heteroconjugate Ig derived proteins are composed of two covalently joined Ig derived proteins. Such Ig derived proteins have, for example, been proposed to target immune system cells to unwanted cells (U.S. Pat. No. 4,676,980), and for treatment of HIV infection (WO 91/00360; WO 92/00373; and EP 03089). Heteroconjugate Ig derived proteins can be made using any convenient cross-linking methods. Suitable cross-linking agents are well known in the art, and are disclosed in U.S. Pat. No. 4,676,980, along with a number of cross-linking techniques.

In a preferred embodiment, at least one anti-COPD-related Ig derived protein or specified portion or variant of the present invention is produced by a cell line, a mixed cell line, an immortalized cell or clonal population of immortalized cells. Immortalized COPD-related producing cells can be produced using suitable methods, for example, fusion of a human Ig derived protein-producing cell and a heteromyeloma or immortalization of an activated human B cell via infection with Epstein Barr virus (Niedbala et al., Hybridoma, 17(3):299-304 (1998); Zanella et al., J Immunol Methods, 156(2):205-215 (1992); Gustafsson et al., Hum Ig derived proteins Hybridomas, 2(1)₂₆-32 (1991)). Preferably, the human anti-human COPD-related Ig derived protein or specified portion or variant is generated by immunization of a transgenic animal (e.g., mouse, rat, hamster, non-human primate, and the like) capable of producing a repertoire of human Ig derived proteins, as described herein and/or as known in the art. Cells that produce a human anti-human COPD-related Ig derived protein can be isolated from such animals and immortalized using suitable methods, such as the methods described herein.

Transgenic mice that can produce a repertoire of human Ig derived proteins that bind to human antigens can be produced by known methods (e.g., but not limited to, U.S. Pat. Nos. 5,770,428, 5,569,825, 5,545,806, 5,625,126, 5,625,825, 5,633,425, 5,661,016 and 5,789,650 issued to Lonberg et al.; Jakobovits et al. WO 98/50433, Jakobovits et al. WO 98/24893, Lonberg et al. WO 98/24884, Lonberg et al. WO 97/13852, Lonberg et al. WO 94/25585, Kucherlapate et al. WO 96/34096, Kucherlapate et al. EP 0463 151 B 1, Kucherlapate et al. EP 0710 719 A1, Surani et al. U.S. Pat. No. 5,545,807, Bruggemann et al. WO 90/04036, Bruggemann et al. EP 0438 474 Bi, Lonberg et al. EP 0814 259 A2, Lonberg et al. GB 2 272 440 A, Lonberg et al. Nature 368:856-859 (1994), Taylor et al., Int. Immunol. 6(4)579-591 (1994), Green et al, Nature Genetics 7:13-21 (1994), Mendez et al., Nature Genetics 15:146-156 (1997), Taylor et al., Nucleic Acids Research 20(23):6287-6295 (1992), Tuaillon et al., Proc Natl Acad Sci USA 90(8)3720-3724 (1993), Lonberg et al., Int Rev Immunol 13(l):65-93 (1995) and Fishwald et al., Nat Biotechnol 14(7):845-851 (1996), which are each entirely incorporated herein by reference). Generally, these mice comprise at least one transgene comprising DNA from at least one human immunoglobulin locus that is functionally rearranged, or which can undergo functional rearrangement. The endogenous immunoglobulin loci in such mice can be disrupted or deleted to eliminate the capacity of the animal to produce Ig derived proteins encoded by endogenous genes.

The term “functionally rearranged,” as used herein refers to a segment of DNA from an immunoglobulin locus that has undergone V(D)J recombination, thereby producing an immunoglobulin gene that encodes an immunoglobulin chain (e.g., heavy chain, light chain), or any portion thereof. A functionally rearranged immunoglobulin gene can be directly or indirectly identified using suitable methods, such as, for example, nucleotide sequencing, hybridization (e.g., Southern blotting, Northern blotting) using probes that can anneal to coding joints between gene segments or enzymatic amplification of immunoglobulin genes (e.g., polymerase chain reaction) with primers that can anneal to coding joints between gene segments. Whether a cell produces an Ig derived protein comprising a particular variable region or a variable region comprising a particular sequence (e.g., at least one CDR sequence) can also be determined using suitable methods. In one example, mRNA can be isolated from an Ig derived protein-producing cell (e.g., a hybridoma or recombinant cell or other suitable source) and used to produce cDNA encoding the Ig derived protein or specified portion or variant thereof. The cDNA can be cloned and sequenced or can be amplified (e.g., by polymerase chain reactionor other known and suitable methods) using a first primer that anneals specifically to a portion of the variable region of interest (e.g., CDR, coding joint) and a second primer that anneals specifically to non-variable region sequences (e.g., C_{H}1, V_{H}).

Screening Ig derived protein or specified portion or variants for specific binding to similar proteins or fragments can be conveniently achieved using peptide display libraries. This method involves the screening of large collections of peptides for individual members having the desired function or structure. Ig derived protein screening of peptide display libraries is well known in the art. The displayed peptide sequences can be from 3 to 5000 or more amino acids in length, frequently from 5-100 amino acids long, and often from about 8 to 25 amino acids long. In addition to direct chemical synthetic methods for generating peptide libraries, several recombinant DNA methods have been described. One type involves the display of a peptide sequence on the surface of a bacteriophage or cell. Each bacteriophage or cell contains the nucleotide sequence encoding the particular displayed peptide sequence. Such methods are described in PCT Patent Publication Nos. 91/17271, 91/18980,91/19818, and 93/08278. Other systems for generating libraries of peptides have aspects of both in vitro chemical synthesis and recombinant methods. See, PCT Patent Publication Nos. 92/05258, 92/14843, and 96/19256. See also, U.S. Pat. Nos. 5,658,754; and 5,643,768. Peptide display libraries, vector, and screening kits are commercially available from such suppliers as Invitrogen (Carlsbad, Calif.), and Cambridge Ig derived protein Technologies (Cambridgeshire, UK). See, e.g., U.S. Pat. Nos. 4,704,692, 4,939,666, 4,946,778, 5,260,203, 5,455,030, 5,5188,89, 5,534,621, 5,656,730, 5,763,733, 5,767,260, 5,856,456, assigned to Enzon; U.S. Pat. Nos. 5,223,409, 5,403,484, 5,571,698, 5,837,500, assigned to Dyax, U.S. Pat. Nos. 5,427,908, 5,580,717, assigned to Affymax; U.S. Pat. No. 5,885,793, assigned to Cambridge Ig derived protein Technologies; U.S. Pat. No. 5,750,373, assigned to Genentech, U.S. Pat. Nos. 5,618,920, 5,595,898, 5,576,195, 5,698,435, 5,693,493, 5,698,417, assigned to Xoma, Colligan, supra; Ausubel, supra; or Sambrook, supra, each of the above patents and publications entirely incorporated herein by reference.

Ig derived proteins, specified portions and variants of the present invention can also be prepared using at least one COPD-related Ig derived protein or specified portion or variant encoding nucleic acid to provide transgenic animals or mammals, such as goats, cows, horses, sheep, and the like, that produce such Ig derived proteins or specified portions or variants in their milk. Such animals can be provided using known methods. See, e.g., but not limited to, U.S. Pat. Nos. 5,827,690; 5,849,992; 4,873,316; 5,849,992; 5,994,616; 5,565,362; 5,304,489, and the like, each of which is entirely incorporated herein by reference.

Ig derived proteins, specified portions and variants of the present invention can additionally be prepared using at least one COPD-related Ig derived protein or specified portion or variant encoding nucleic acid to provide transgenic plants and cultured plant cells (e.g., but not limited to tobacco and maize) that produce such Ig derived proteins, specified portions or variants in the plant parts or in cells cultured therefrom. As a non-limiting example, transgenic tobacco leaves expressing recombinant proteins have been successfully used to provide large amounts of recombinant proteins, e.g., using an inducible promoter. See, e.g., Cramer et al., Curr. Top. Microbol. Immunol. 240:95-118 (1999) and references cited therein. Also, transgenic maize have been used to express mammalian proteins at commercial production levels, with biological activities equivalent to those produced in other recombinant systems or purified from natural sources. See, e.g., Hood et al., Adv. Exp. Med. Biol. 464:127-147 (1999) and references cited therein. Ig derived proteins have also been produced in large amounts from transgenic plant seeds including Ig derived protein fragments, such as single chain Ig derived proteins (scFv's), including tobacco seeds and potato tubers. See, e.g., Conrad et al., Plant Mol. Biol. 38:101-109 (1998) and reference cited therein. Thus, Ig derived proteins, specified portions and variants of the present invention can also be produced using transgenic plants, according to know methods. See also, e.g., Fischer et al., Biotechnol. Appl. Biochem. 30:99-108 (October, 1999), Ma et al., Trends Biotechnol. 13:522-7 (1995); Ma et al., Plant Physiol. 109:341-6 (1995); Whitelam et al., Biochem. Soc. Trans. 22:940-944 (1994); and references cited therein. See, also generally for plant expression of Ig derived proteins, but not limited to, Each of the above references is entirely incorporated herein by reference.

The Ig derived proteins of the invention can bind human COPD-related with a wide range of affinities (K_{D}). In a preferred embodiment, at least one human mAb of the present invention can optionally bind human COPD-related with high affinity. For example, a human mAb can bind human COPD-related with a K_{D} equal to or less than about 10⁻⁹ M or, more preferably, with a K_{D} equal to or less than about 0.1-9.9 (or any range or value therein)×10⁻¹⁰M, 10⁻¹¹, 10⁻¹², 10⁻¹³ or any range or value therein.

The affinity or avidity of an Ig derived protein for an antigen can be determined experimentally using any suitable method. (See, for example, Berzofsky, et al., “Ig derived protein-Antigen Interactions,” In Fundamental Immunology, Paul, W. E., Ed., Raven Press: New York, N.Y. (1984); Kuby, Janis Immunology, W. H. Freeman and Company: New York, N.Y. (1992); and methods described herein). The measured affinity of a particular Ig derived protein-antigen interaction can vary if measured under different conditions (e.g., salt concentration, pH). Thus, measurements of affinity and other antigen-binding parameters (e.g., K_{D}, Kₐ, K_{d}) are preferably made with standardized solutions of Ig derived protein and antigen, and a standardized buffer, such as the buffer described herein.

Nucleic Acid Molecules

Using the information provided herein, such as the nucleotide sequences encoding at least 90-100% of the contiguous amino acids of at least one of COPD related Ig derived protein of the present invention, specified fragments, variants or consensus sequences thereof, or a deposited vector comprising at least one of these sequences, a nucleic acid molecule of the present invention encoding at least one COPD-related Ig derived protein or specified portion or variant can be obtained using methods described herein or as known in the art.

Nucleic acid molecules of the present invention can be in the form of RNA, such as mRNA, hnRNA, tRNA or any other form, or in the form of DNA, including, but not limited to, cDNA and genomic DNA obtained by cloning or produced synthetically, or any combinations thereof. The DNA can be triple-stranded, double-stranded or single-stranded, or any combination thereof. Any portion of at least one strand of the DNA or RNA can be the coding strand, also known as the sense strand, or it can be the non-coding strand, also referred to as the anti-sense strand.

Isolated nucleic acid molecules of the present invention can include nucleic acid molecules comprising an open reading frame (ORF), optionally with one or more introns, e.g., but not limited to, at least one specified portion of at least one CDR, as CDR1, CDR2 and/or CDR3 of at least one heavy chain or light chain, respectively; nucleic acid molecules comprising the coding sequence for a COPD-related Ig derived protein or specified portion or variant; and nucleic acid molecules which comprise a nucleotide sequence substantially different from those described above but which, due to the degeneracy of the genetic code, still encode at least one COPD-related Ig derived protein as described herein and/or as known in the art. Of course, the genetic code is well known in the art. Thus, it would be routine for one skilled in the art to generate such degenerate nucleic acid variants that code for specific COPD-related Ig derived protein or specified portion or variants of the present invention. See, e.g., Ausubel, et al., supra, and such nucleic acid variants are included in the present invention.

In another aspect, the invention provides isolated nucleic acid molecules encoding a(n) COPD-related Ig derived protein or specified portion or variant having an amino acid sequence as encoded by the nucleic acid contained in the plasmid deposited as designated clone names ______ and ATCC Deposit Nos. ______ respectively, deposited on ______.

As indicated herein, nucleic acid molecules of the present invention which comprise a nucleic acid encoding a COPD-related Ig derived protein or specified portion or variant can include, but are not limited to, those encoding the amino acid sequence of an Ig derived protein fragment, by itself; the coding sequence for the entire Ig derived protein or a portion thereof; the coding sequence for an Ig derived protein, fragment or portion, as well as additional sequences, such as the coding sequence of at least one signal leader or fusion peptide, with or without the aforementioned additional coding sequences, such as at least one intron, together with additional, non-coding sequences, including but not limited to, non-coding 5′ and 3′ sequences, such as the transcribed, non-translated sequences that play a role in transcription, mRNA processing, including splicing and polyadenylation signals (for example—ribosome binding and stability of mRNA); an additional coding sequence that codes for additional amino acids, such as those that provide additional functionalities. Thus, the sequence encoding an Ig derived protein or specified portion or variant can be fused to a marker sequence, such as a sequence encoding a peptide that facilitates purification of the fused Ig derived protein or specified portion or variant comprising an Ig derived protein fragment or portion.

Polynucleotides Which Selectively Hybridize to a Polynucleotide as Described Herein

The present invention provides isolated nucleic acids that hybridize under selective hybridization conditions to a polynucleotide encoding a COPD related Ig derived protein of the present invention. Thus, the polynucleotides of this embodiment can be used for isolating, detecting, and/or quantifying nucleic acids comprising such polynucleotides. For example, polynucleotides of the present invention can be used to identify, isolate, or amplify partial or full-length clones in a deposited library. In some embodiments, the polynucleotides are genomic or cDNA sequences isolated, or otherwise complementary to, a cDNA from a human or mammalian nucleic acid library.

Preferably, the cDNA library comprises at least 80% full-length sequences, preferably at least 85% or 90% full-length sequences, and more preferably at least 95% full-length sequences. The cDNA libraries can be normalized to increase the representation of rare sequences. Low or moderate stringency hybridization conditions are typically, but not exclusively, employed with sequences having a reduced sequence identity relative to complementary sequences. Moderate and high stringency conditions can optionally be employed for sequences of greater identity. Low stringency conditions allow selective hybridization of sequences having about 70% sequence identity and can be employed to identify orthologous or paralogous sequences.

Optionally, polynucleotides of this invention will encode at least a portion of an Ig derived protein or specified portion or variant encoded by the polynucleotides described herein. The polynucleotides of this invention embrace nucleic acid sequences that can be employed for selective hybridization to a polynucleotide encoding an Ig derived protein or specified portion or variant of the present invention. See, e.g., Ausubel, supra; Colligan, supra, each entirely incorporated herein by reference.

Construction of Nucleic Acids

The isolated nucleic acids of the present invention can be made using (a) recombinant methods, (b) synthetic techniques, (c) purification techniques, or combinations thereof, as well-known in the art.

The nucleic acids can conveniently comprise sequences in addition to a polynucleotide of the present invention. For example, a multi-cloning site comprising one or more endonuclease restriction sites can be inserted into the nucleic acid to aid in isolation of the polynucleotide. Also, translatable sequences can be inserted to aid in the isolation of the translated polynucleotide of the present invention. For example, a hexa-histidine marker sequence provides a convenient means to purify the proteins of the present invention. The nucleic acid of the present invention—excluding the coding sequence—is optionally a vector, adapter, or linker for cloning and/or expression of a polynucleotide of the present invention.

Additional sequences can be added to such cloning and/or expression sequences to optimize their function in cloning and/or expression, to aid in isolation of the polynucleotide, or to improve the introduction of the polynucleotide into a cell. Use of cloning vectors, expression vectors, adapters, and linkers is well known in the art. (See, e.g., Ausubel, supra; or Sambrook, supra)

Recombinant Methods for Constructing Nucleic Acids

The isolated nucleic acid compositions of this invention, such as RNA, cDNA, genomic DNA, or any combination thereof, can be obtained from biological sources using any number of cloning methodologies known to those of skill in the art. In some embodiments, oligonucleotide probes that selectively hybridize, under stringent conditions, to the polynucleotides of the present invention are used to identify the desired sequence in a cDNA or genomic DNA library. The isolation of RNA, and construction of cDNA and genomic libraries, is well known to those of ordinary skill in the art. (See, e.g., Ausubel, supra; or Sambrook, supra)

Nucleic Acid Screening and Isolation Methods

A cDNA or genomic library can be screened using a probe based upon the sequence of a polynucleotide of the present invention, such as those disclosed herein. Probes can be used to hybridize with genomic DNA or cDNA sequences to isolate homologous genes in the same or different organisms. Those of skill in the art will appreciate that various degrees of stringency of hybridization can be employed in the assay; and either the hybridization or the wash medium can be stringent. As the conditions for hybridization become more stringent, there must be a greater degree of complementarity between the probe and the target for duplex formation to occur. The degree of stringency can be controlled by one or more of temperature, ionic strength, pH and the presence of a partially denaturing solvent such as formamide. For example, the stringency of hybridization is conveniently varied by changing the polarity of the reactant solution through, for example, manipulation of the concentration of formamide within the range of 0% to 50%. The degree of complementarity (sequence identity) required for detectable binding will vary in accordance with the stringency of the hybridization medium and/or wash medium. The degree of complementarity will optimally be 100%, or 90-100%, or any range or value therein. However, it should be understood that minor sequence variations in the probes and primers can be compensated for by reducing the stringency of the hybridization and/or wash medium.

Methods of amplification of RNA or DNA are well known in the art and can be used according to the present invention without undue experimentation, based on the teaching and guidance presented herein.

Known methods of DNA or RNA amplification include, but are not limited to, polymerase chain reaction (PCR) and related amplification processes (see, e.g., U.S. Pat. Nos. 4,683,195, 4,683,202, 4,800,159, 4,965,188, to Mullis, et al.; U.S. Pat. Nos. 4,795,699 and 4,921,794 to Tabor, et al; U.S. Pat. No. 5,142,033 to Innis; U.S. Pat. No. 5,122,464 to Wilson, et al.; U.S. Pat. No. 5,091,310 to Innis; U.S. Pat. No. 5,066,584 to Gyllensten, et al; U.S. Pat. No. 4,889,818 to Gelfand, et al; U.S. Pat. No. 4,994,370 to Silver, et al; U.S. Pat. No. 4,766,067 to Biswas; 4,656,134 to Ringold) and RNA mediated amplification that uses anti-sense RNA to the target sequence as a template for double-stranded DNA synthesis (U.S. Pat. No. 5,130,238 to Malek, et al, with the tradename NASBA), the entire contents of which references are incorporated herein by reference. (See, e.g., Ausubel, supra; or Sambrook, supra.)

For instance, polymerase chain reaction (PCR) technology can be used to amplify the sequences of polynucleotides of the present invention and related genes directly from genomic DNA or cDNA libraries. PCR and other in vitro amplification methods can also be useful, for example, to clone nucleic acid sequences that code for proteins to be expressed, to make nucleic acids to use as probes for detecting the presence of the desired mRNA in samples, for nucleic acid sequencing, or for other purposes. Examples of techniques sufficient to direct persons of skill through in vitro amplification methods are found in Berger, supra, Sambrook, supra, and Ausubel, supra, as well as Mullis, et al., U.S. Pat. No. 4,683,202 (1987); and Innis, et al., PCR Protocols A Guide to Methods and Applications, Eds., Academic Press Inc., San Diego, Calif. (1990). Commercially available kits for genomic PCR amplification are known in the art. See, e.g., Advantage-GC Genomic PCR Kit (Clontech). The T4 gene 32 protein (Boehringer Mannheim) can be used to improve yield of long PCR products.

Synthetic Methods for Constructing Nucleic Acids

The isolated nucleic acids of the present invention can also be prepared by direct chemical synthesis by known methods (see, e.g., Ausubel, et al., supra). Chemical synthesis generally produces a single-stranded oligonucleotide, which can be converted into double-stranded DNA by hybridization with a complementary sequence, or by polymerization with a DNA polymerase using the single strand as a template. One of skill in the art will recognize that while chemical synthesis of DNA can be limited to sequences of about 100 or more bases, longer sequences can be obtained by the ligation of shorter sequences.

Recombinant Expression Cassettes

The present invention further provides recombinant expression cassettes comprising a nucleic acid of the present invention. A nucleic acid sequence of the present invention, for example a cDNA or a genomic sequence encoding an Ig derived protein or specified portion or variant of the present invention, can be used to construct a recombinant expression cassette that can be introduced into at least one desired host cell. A recombinant expression cassette will typically comprise a polynucleotide of the present invention operably linked to transcriptional initiation regulatory sequences that will direct the transcription of the polynucleotide in the intended host cell. Both heterologous and non-heterologous (i.e., endogenous) promoters can be employed to direct expression of the nucleic acids of the present invention.

In some embodiments, isolated nucleic acids that serve as promoter, enhancer, or other elements can be introduced in the appropriate position (upstream, downstream or in intron) of a non-heterologous form of a polynucleotide of the present invention so as to up or down regulate expression of a polynucleotide of the present invention. For example, endogenous promoters can be altered in vivo or in vitro by mutation, deletion and/or substitution.

A polynucleotide of the present invention can be expressed in either sense or anti-sense orientation as desired. It will be appreciated that control of gene expression in either sense or anti-sense orientation can have a direct impact on the observable characteristics.

Another method of suppression is sense suppression. Introduction of nucleic acid configured in the sense orientation has been shown to be an effective means by which to block the transcription of target genes.

A variety of cross-linking agents, alkylating agents and radical generating species as pendant groups on polynucleotides of the present invention can be used to bind, label, detect and/or cleave nucleic acids. Knorre, et al., Biochimie 67:785-789 (1985); Vlassov, et al., Nucleic Acids Res. 14:4065-4076 (1986); Iverson and Dervan, J. Am. Chem. Soc. 109:1241-1243 (1987); Meyer, et al., J. An. Chem. Soc. 111:8517-8519 (1989); Lee, et al., Biochemistry 27:3197-3203 (1988); Home, et al., J. Am. Chem. Soc. 112:2435-2437 (1990); Webb and Matteucci, J. Am. Chem. Soc. 108:2764-2765 (1986); Nucleic Acids Res. 14:7661-7674 (1986); Feteritz, et al., J. Am. Chem. Soc. 113:4000 (1991). Various compounds to bind, detect, label, and/or cleave nucleic acids are known in the art. See, for example, U.S. Pat. Nos. 5,543,507; 5,672,593; 5,484,908; 5,256,648; and 5,681941, each entirely incorporated herein by reference.

Vectors And Host Cells

The present invention also relates to vectors that include isolated nucleic acid molecules of the present invention, host cells that are genetically engineered with the recombinant vectors, and the production of at least one COPD-related Ig derived protein or specified portion or variant by recombinant techniques, as is well known in the art. See, e.g., Sambrook, et al., supra; Ausubel, et al., supra, each entirely incorporated herein by reference.

The polynucleotides can optionally be joined to a vector containing a selectable marker for propagation in a host. Generally, a plasmid vector is introduced in a precipitate, such as a calcium phosphate precipitate, or in a complex with a charged lipid. If the vector is a virus, it can be packaged in vitro using an appropriate packaging cell line and then transduced into host cells.

The DNA insert should be operatively linked to an appropriate promoter. The expression constructs will further contain sites for transcription initiation, termination and, in the transcribed region, a ribosome binding site for translation. The coding portion of the mature transcripts expressed by the constructs will preferably include a translation initiating at the beginning and a termination codon (e.g., UAA, UGA or UAG) appropriately positioned at the end of the mRNA to be translated, with UAA and UAG preferred for mammalian or eukaryotic cell expression.

Expression vectors will preferably but optionally include at least one selectable marker. Such markers include, e.g., but not limited to, methotrexate (MTX), dihydrofolate reductase (DHFR, U.S. Pat. Nos. 4,399,216; 4,634,665; 4,656,134; 4,956,288; 5,149,636; 5,179,017, ampicillin, neomycin (G418), mycophenolic acid, or glutamine synthetase (GS, U.S. Pat. Nos. 5,122,464; 5,770,359; 5,827,739) resistance for eukaryotic cell culture, and tetracycline or ampicillin resistance genes for culturing in E. coli and other bacteria or prokaryotics (the above patents are entirely incorporated hereby by reference). Appropriate culture mediums and conditions for the above-described host cells are known in the art. Suitable vectors will be readily apparent to the skilled artisan. Introduction of a vector construct into a host cell can be effected by calcium phosphate transfection, DEAE-dextran mediated transfection, cationic lipid-mediated transfection, electroporation, transduction, infection or other known methods. Such methods are described in the art, such as Sambrook, supra, Chapters 1-4 and 16-18; Ausubel, supra, Chapters 1, 9, 13, 15, 16.

At least one Ig derived protein or specified portion or variant of the present invention can be expressed in a modified form, such as a fusion protein, and can include not only secretion signals, but also additional heterologous functional regions. For instance, a region of additional amino acids, particularly charged amino acids, can be added to the N-terminus of an Ig derived protein or specified portion or variant to improve stability and persistence in the host cell, during purification, or during subsequent handling and storage. Also, peptide moieties can be added to an Ig derived protein or specified portion or variant of the present invention to facilitate purification. Such regions can be removed prior to final preparation of an Ig derived protein or at least one fragment thereof. Such methods are described in many standard laboratory manuals, such as Sambrook, supra, Chapters 17.29-17.42 and 18.1-18.74; Ausubel, supra, Chapters 16, 17 and 18.

Those of ordinary skill in the art are knowledgeable in the numerous expression systems available for expression of a nucleic acid encoding a protein of the present invention.

Alternatively, nucleic acids of the present invention can be expressed in a host cell by turning on (by manipulation) in a host cell that contains endogenous DNA encoding an Ig derived protein or specified portion or variant of the present invention. Such methods are well known in the art, e.g., as described in U.S. Pat. Nos. 5,580,734, 5,641,670, 5,733,746, and 5,733,761, entirely incorporated herein by reference.

Illustrative of cell cultures useful for the production of the Ig derived proteins, specified portions or variants thereof, are mammalian cells. Mammalian cell systems often will be in the form of monolayers of cells although mammalian cell suspensions or bioreactors can also be used. A number of suitable host cell lines capable of expressing intact glycosylated proteins have been developed in the art and include the COS-1 (e.g., ATCC CRL 1650), COS-7 (e.g., ATCC CRL-1651), HEK293, BHK21 (e.g., ATCC CRL-10), CHO (e.g., ATCC CRL 1610) and BSC-1 (e.g., ATCC CRL-26) cell lines, Cos-7 cells, CHO cells, hep G2 cells, P3X63Ag8.653, SP2/0-Ag14, 293 cells, HeLa cells and the like, which are readily available from, for example, American Type Culture Collection, Manassas, Va. Preferred host cells include cells of lymphoid origin such as myeloma and lymphoma cells. Particularly preferred host cells are P3X63Ag8.653 cells (ATCC Accession Number CRL-1580) and SP2/0-Ag14 cells (ATCC Accession Number CRL-1851). In a particularly preferred embodiment, the recombinant cell is a P3X63Ab8.653 or a SP2/0-Ag14 cell.

Expression vectors for these cells can include one or more of the following expression control sequences, such as, but not limited to an origin of replication; a promoter (e.g., late or early SV40 promoters, the CMV promoter (U.S. Pat. Nos. 5,168,062; 5,385,839), an HSV tk promoter, a pgk (phosphoglycerate kinase) promoter, an EF-1 alpha promoter (U.S. Pat. No. 5,266,491), at least one human immunoglobulin promoter; an enhancer, and/or processing information sites, such as ribosome binding sites, RNA splice sites, polyadenylation sites (e.g., an SV40 large T Ag poly A addition site), and transcriptional terminator sequences. See, e.g., Ausubel et al., supra; Sambrook, et al., supra. Other cells useful for production of nucleic acids or proteins of the present invention are known and/or available, for instance, from the American Type Culture Collection Catalogue of Cell Lines and Hybridomas (www.atcc.org) or other known or commercial sources.

When eukaryotic host cells are employed, polyadenlyation or transcription terminator sequences are typically incorporated into the vector. An example of a terminator sequence is the polyadenlyation sequence from the bovine growth hormone gene. Sequences for accurate splicing of the transcript can also be included. An example of a splicing sequence is the VP1 intron from SV40 (Sprague, et al., J. Virol. 45:773-781 (1983)). Additionally, gene sequences to control replication in the host cell can be incorporated into the vector, as known in the art.

Purification of an Ig derived protein or Specified Portion or Variant Thereof

A COPD-related Ig derived protein or specified portion or variant can be recovered and purified from recombinant cell cultures by well-known methods including, but not limited to, protein A purification, ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. High performance liquid chromatography (“HPLC”) can also be employed for purification. See e.g., Colligan, Current Protocols in Immunology, or Current Protocols in Protein Science, John Wiley & Sons, NY, N.Y., (1997-2001), e.g., Chapters 1, 4, 6, 8, 9, 10, each entirely incorporated herein by reference.

Ig derived proteins or specified portions or variants of the present invention include naturally purified products, products of chemical synthetic procedures, and products produced by recombinant techniques from a eukaryotic host, including, for example, yeast, higher plant, insect and mammalian cells. Depending upon the host employed in a recombinant production procedure, the Ig derived protein or specified portion or variant of the present invention can be glycosylated or can be non-glycosylated, with glycosylated preferred. Such methods are described in many standard laboratory manuals, such as Sambrook, supra, Sections 17.37-17.42; Ausubel, supra, Chapters 10, 12, 13, 16, 18 and 20, Colligan, Protein Science, supra, Chapters 12-14, all entirely incorporated herein by reference.

COPD-related Ig Derived Proteins, Fragments and/or Variants

The isolated Ig derived proteins of the present invention comprise an Ig derived protein or specified portion or variant encoded by any one of the polynucleotides of the present invention as discussed more fully herein, or any isolated or prepared Ig derived protein or specified portion or variant thereof.

Preferably, the human Ig derived protein or antigen-binding fragment binds human COPD-related and, thereby substantially neutralizes the biological activity of the protein. An Ig derived protein, or specified portion or variant thereof, that partially or preferably substantially neutralizes at least one biological activity of at least one COPD-related protein or fragment can bind the protein or fragment and thereby inhibit activitys mediated through the binding of COPD-related to the COPD-related receptor or through other COPD-related-dependent or mediated mechanisms. As used herein, the term “neutralizing Ig derived protein” refers to an Ig derived protein that can inhibit an COPD-related-dependent activity by about 20-120%, preferably by at least about 60, 70, 80, 90, 91, 92, 93, 94, 95, 96, 97, 98, 99, 100% or more depending on the assay. The capacity of an COPD-related Ig derived protein or specified portion or variant to inhibit an COPD-related-dependent activity is preferably assessed by at least one suitable COPD-related Ig derived protein or protein assay, as described herein and/or as known in the art. A human Ig derived protein or specified portion or variant of the invention can be of any class (IgG, IgA, IgM, IgE, IgD, etc.) or isotype and can comprise a kappa or lambda light chain. In one embodiment, the human Ig derived protein or specified portion or variant comprises an IgG heavy chain or defined fragment, for example, at least one of isotypes, IgG1, IgG2, IgG3 or IgG4. Ig derived proteins of this type can be prepared by employing a transgenic mouse or other trangenic non-human mammal comprising at least one human light chain (e.g., IgG, IgA and IgM (e.g., γ1, γ2, γ3, γ4) transgenes as described herein and/or as known in the art. In another embodiment, the anti-human COPD-related human Ig derived protein or specified portion or variant thereof comprises an IgG1 heavy chain and a IgG 1 light chain.

At least one Ig derived protein or specified portion or variant of the invention binds at least one specified epitope specific to at least one COPD-related protein, subunit, fragment, portion or any combination thereof. The at least one epitope can comprise at least one Ig derived protein binding region that comprises at least one portion of said protein, which epitope is preferably comprised of at least one extracellular, soluble, hydrophillic, external or cytoplasmic portion of said protein. As non-limiting examples, (a) a COPD-related Ig derived protein or specified portion or variant specifically binds at least one epitope comprising at least 1-3, to the entire amino acid sequence, selected from the group consisting of a human tissue necrosis factor alpha (TNF), an interleukin-6 (IL-6), an interleukin-8 (IL-8); an epidermal growth factor (EGF); a CD-8; or a CD-18; (b) the at least one specified epitope can comprise any combination of at least one amino acid sequence of at least 1-3 amino acids to the entire specified portion of contiguous amino acids of the sequences selected from the group consisting of: from 1-80 to 80-157 of SEQ ID NO:1; from 77-116 to 117-233 of SEQ ID NO:2; from 28-106 to 107-212 of SEQ ID NO:3; from 21-50 to 51-99 of SEQ ID NO:4; from 23-605 to 606-1207 of SEQ ID NO:5; from 22-118 to 119-235 of SEQ ID NO:6; from 1-23 to 24-45 of SEQ ID NO:7; from 1-19 to 20-37 of SEQ ID NO:8; from 22-105 to 106-210 of SEQ ID NO:9; from 1-123 to 124-246 of SEQ ID NO:10; from 1-40 to 40-80 of SEQ ID NO:11; from 23-385 to 386-769 of SEQ ID NO: 12; or (c) the at least one specified epitope can comprise any combination of at least one amino acid sequence of at least 1-3 amino acids to the entire specified portion of contiguous amino acids of the sequences selected from the group consisting of: from 1-50 to 100-157 of SEQ ID NO: 1; from 77-122 and 145-233 of SEQ ID NO:2; from 21-40 to 200-212 of SEQ ID NO:3; from 21-40 to 66-99 of SEQ ID NO:4; from 23-150 to 805-1022 of SEQ ID NO:5; from 22-100 to 200-235, 22-171, 209-235, 36-117, 118-182, 206-235 of SEQ ID NO:6; from 1-10 to 35-45 of SEQ ID NO:7; from 1-10 to 30-37 of SEQ ID NO:8; from 22-105 to 106-164, 22-123, 124-135, 136-164 of SEQ ID NO:9; from 1-50 to 200-246 of SEQ ID NO:10; from 1-20 to 60-80 of SEQ ID NO:11; from 23-499 to 500-670, 445-631, 459-540, 541-627 of SEQ ID NO: 12.

Alternatively, a COPD related protein, Ig derived protein or specified portion or variant comprises at least COPD-related protein binding region selected from at least 1-3 amino acids selected from the group consisting of a human tissue necrosis factor alpha (TNF) ligand or receptor, an interleukin-6 (IL-6) receptor or ligand, an interleukin-8 (IL-8) receptor or ligand; an epidermal growth factor (EGF) receptor or ligand; a CD-8 receptor or ligand; or a CD-18 receptor or ligand. As a non-limiting example, the COPD-related Ig derived protein or specified portion or variant comprises at least COPD-related protein binding region selected from at least 1-3 amino acids selected from the group consisting of 22-455 of SEQ ID NO: 13; 1-53 of SEQ ID NO: 14; 1-350 of SEQ ID NO:15; 1-360 of SEQ ID NO:16; and/or 25-645 of SEQ ID NO:17.

Generally, the human Ig derived protein or antigen-binding fragment of the present invention will comprise an antigen-binding region that comprises at least one human complementarity determining region (CDR1, CDR2 and CDR3) or variant of at least one heavy chain variable region and at least one human complementarity determining region (CDR1, CDR2 and CDR3) or variant of at least one light chain variable region. As a non-limiting example, the Ig derived protein or antigen-binding portion or variant can comprise at least one of the heavy chain CDR3, and/or a light chain CDR3. In a particular embodiment, the Ig derived protein or antigen-binding fragment can have an antigen-binding region that comprises at least a portion of at least one heavy chain CDR (i.e., CDR1, CDR2 and/or CDR3) having the amino acid sequence of the corresponding CDRs 1, 2 and/or 3. In another particular embodiment, the Ig derived protein or antigen-binding portion or variant can have an antigen-binding region that comprises at least a portion of at least one light chain CDR (i.e., CDR1, CDR2 and/or CDR3) having the amino acid sequence of the corresponding CDRs 1, 2 and/or 3. Such Ig derived proteins can be prepared by chemically joining together the various portions (e.g., CDRs, framework) of the Ig derived protein using conventional techniques, by preparing and expressing a (i.e., one or more) nucleic acid molecule that encodes the Ig derived protein using conventional techniques of recombinant DNA technology or by using any other suitable method.

The anti-human COPD-related human Ig derived protein can comprise at least one of a heavy or light chain variable region having a defined amino acid sequence. For example, in a preferred embodiment, the human anti-human COPD-related Ig derived protein comprises at least one of at least one heavy chain variable region and/or at least one light chain variable region. Human Ig derived proteins that bind to human COPD-related and that comprise a defined heavy or light chain variable region can be prepared using suitable methods, such as phage display (Katsube, Y., et al., Int J. Mol. Med, 1(5):863-868 (1998)) or methods that employ transgenic animals, as known in the art and/or as described herein. For example, a transgenic mouse, comprising a functionally rearranged human immunoglobulin heavy chain transgene and a transgene comprising DNA from a human immunoglobulin light chain locus that can undergo functional rearrangement, can be immunized with human COPD-related or a fragment thereof to elicit the production of Ig derived proteins. If desired, the Ig derived protein producing cells can be isolated and hybridomas or other immortalized Ig derived protein-producing cells can be prepared as described herein and/or as known in the art. Alternatively, the Ig derived protein, specified portion or variant can be expressed using the encoding nucleic acid or portion thereof in a suitable host cell.

The invention also relates to Ig derived proteins, antigen-binding fragments, immunoglobulin chains and CDRs comprising amino acids in a sequence that is substantially the same as an amino acid sequence described herein. Preferably, such Ig derived proteins or antigen-binding fragments and Ig derived proteins comprising such chains or CDRs can bind human COPD-related with high affinity (e.g., K_{D} less than or equal to about 10⁻⁹ M). Amino acid sequences that are substantially the same as the sequences described herein include sequences comprising conservative amino acid substitutions, as well as amino acid deletions and/or insertions. A conservative amino acid substitution refers to the replacement of a first amino acid by a second amino acid that has chemical and/or physical properties (e.g, charge, structure, polarity, hydrophobicity/hydrophilicity) that are similar to those of the first amino acid. Conservative substitutions include replacement of one amino acid by another within the following groups: lysine (K), arginine (R) and histidine (H); aspartate (D) and glutamate (E); asparagine (N), glutamine (Q), serine (S), threonine (T), tyrosine (Y), K, R, H, D and E; alanine (A), valine (V), leucine (L), isoleucine (I), proline (P), phenylalanine (F), tryptophan (W), methionine (M), cysteine (C) and glycine (G); F, W and Y; C, S and T.

Amino Acid Codes

The amino acids that make up COPD-related Ig derived proteins or specified portions or variants of the present invention are often abbreviated. The amino acid designations can be indicated by designating the amino acid by its single letter code, its three letter code, name, or three nucleotide codon(s) as is well understood in the art (see Alberts, B., et al., Molecular Biology of The Cell, Third Ed., Garland Publishing, Inc., New York, 1994):

| SINGLE LETTER | THREE LETTER | | THREE NUCLEOTIDE |
|---|---|---|---|
| CODE | CODE | NAME | CODON(S) |
| A | Ala | Alanine | GCA, GCC, GCG, GCU |
| C | Cys | Cysteine | UGC, UGU |
| D | Asp | Aspartic acid | GAC, GAU |
| E | Glu | Glutamic acid | GAA, GAG |
| F | Phe | Phenylanine | UUC, UUU |
| G | Gly | Glycine | GGA, GGC, GGG, GGU |
| H | His | Histidine | CAC, CAU |
| I | Ile | Isoleucine | AUA, AUC, AUU |
| K | Lys | Lysine | AAA, AAG |
| L | Leu | Leucine | UUA, UUG, CUA, CUC, |
| | | | CUG, CUU |
| M | Met | Methionine | AUG |
| N | Asn | Asparagine | AAC, AAU |
| P | Pro | Proline | CCA, CCC, CCG, CCU |
| Q | Gln | Glutamine | CAA, CAG |
| R | Arg | Arginine | AGA, AGG, CGA, CGC, |
| | | | CGG, CGU |
| S | Ser | Serine | AGC, AGU, UCA, UCC, |
| | | | UCG, UCU |
| T | Thr | Threonine | ACA, ACC, ACG, ACU |
| V | Val | Valine | GUA, GUC, GUG, GUU |
| W | Trp | Tryptophan | UGG |
| Y | Tyr | Tyrosine | UAC, UAU |

A COPD-related Ig derived protein or specified portion or variant of the present invention can include one or more amino acid substitutions, deletions or additions, either from natural mutations or human manipulation, as specified herein.

Of course, the number of amino acid substitutions a skilled artisan would make depends on many factors, including those described above. Generally speaking, the number of amino acid substitutions, insertions or deletions for any given COPD-related polypeptide will not be more than 40, 30, 20, 19,18, 17, 16, 15, 14, 13, 12, 11, 10, 9, 8, 7, 6, 5, 4, 3, 2, 1, such as 1-30 or any range or value therein, as specified herein.

Amino acids in a COPD-related Ig derived protein or specified portion or variant of the present invention that are essential for function can be identified by methods known in the art, such as site-directed mutagenesis or alanine-scanning mutagenesis (e.g., Ausubel, supra, Chapters 8, 15; Cunningham and Wells, Science 244:1081-1085 (1989)). The latter procedure introduces single alanine mutations at every residue in the molecule. The resulting mutant molecules are then tested for biological activity, such as, but not limited to at least one COPD-related neutralizing activity. Sites that are critical for Ig derived protein or specified portion or variant binding can also be identified by structural analysis such as crystallization, nuclear magnetic resonance or photoaffinity labeling (Smith, et al., J. Mol. Biol. 224:899-904 (1992) and de Vos, et al., Science 255:306-312 (1992)).

COPD-related Ig derived proteins or specified portions or variants of the present invention can include, but are not limited to, at least one portion, sequence or combination selected from 5 to all of the contiguous amino acids of at least one of SEQ ID NOS:7, 8, 9, 10, 11, 12.

A(n) COPD-related Ig derived protein or specified portion or variant can further optionally comprise a polypeptide of at least one of 1-50% of the contiguous amino acids of at least one of SEQ ID NOS: 13, 14, 15, 16.

The Ig derived proteins or specified portions or variants of the present invention, or specified variants thereof, can comprise any number of contiguous amino acid residues from an Ig derived protein or specified portion or variant of the present invention, wherein that number is selected from the group of integers consisting of from 10-100% of the number of contiguous residues in a COPD-related Ig derived protein or specified portion or variant. Optionally, this subsequence of contiguous amino acids is at least about 10, 20, 30, 40, 50, 60, 70, 80, 90, 100, 110, 120, 130, 140, 150, 160, 170, 180, 190,200, 210,220, 230,240, 250 or more amino acids in length, or any range or value therein. Further, the number of such subsequences can be any integer selected from the group consisting of from 1 to 20, such as at least 2, 3, 4, or 5.

As those of skill will appreciate, the present invention includes at least one biologically active Ig derived protein or specified portion or variant of the present invention. Biologically active Ig derived proteins or specified portions or variants have a specific activity at least 20%, 30%, or 40%, and preferably at least 50%, 60%, or 70%, and most preferably at least 80%, 90%, or 95%-1000% of that of the native (non-synthetic), endogenous or related and known Ig derived protein or specified portion or variant. Methods of assaying and quantifying measures of enzymatic activity and substrate specificity, are well known to those of skill in the art.

In another aspect, the invention relates to human Ig derived proteins and antigen-binding fragments, as described herein, which are modified by the covalent attachment of an organic moiety. Such modification can produce an Ig derived protein or antigen-binding fragment with improved pharmacokinetic properties (e.g., increased in vivo serum half-life). The organic moiety can be a linear or branched hydrophilic polymeric group, fatty acid group, or fatty acid ester group. In particular embodiments, the hydrophilic polymeric group can have a molecular weight of about 800 to about 120,000 Daltons and can be a polyalkane glycol (e.g., polyethylene glycol (PEG), polypropylene glycol (PPG)), carbohydrate polymer, amino acid polymer or polyvinyl pyrolidone, and the fatty acid or fatty acid ester group can comprise from about eight to about forty carbon atoms.

The modified Ig derived proteins and antigen-binding fragments of the invention can comprise one or more organic moieties that are covalently bonded, directly or indirectly, to the Ig derived protein or specified portion or variant. Each organic moiety that is bonded to an Ig derived protein or antigen-binding fragment of the invention can independently be a hydrophilic polymeric group, a fatty acid group or a fatty acid ester group. As used herein, the term “fatty acid” encompasses mono-carboxylic acids and di-carboxylic acids. A “hydrophilic polymeric group,” as the term is used herein, refers to an organic polymer that is more soluble in water than in octane. For example, polylysine is more soluble in water than in octane. Thus, an Ig derived protein modified by the covalent attachment of polylysine is encompassed by the invention. Hydrophilic polymers suitable for modifying Ig derived proteins of the invention can be linear or branched and include, for example, polyalkane glycols (e.g., PEG, monomethoxy-polyethylene glycol (mPEG), PPG and the like), carbohydrates (e.g., dextran, cellulose, oligosaccharides, polysaccharides and the like), polymers of hydrophilic amino acids (e.g., polylysine, polyarginine, polyaspartate and the like), polyalkane oxides (e.g., polyethylene oxide, polypropylene oxide and the like) and polyvinyl pyrolidone. Preferably, the hydrophilic polymer that modifies the Ig derived protein of the invention has a molecular weight of about 800 to about 150,000 Daltons as a separate molecular entity. For example PEG₅₀₀₀ and PEG_{20,000}, wherein the subscript is the average molecular weight of the polymer in Daltons, can be used.

The hydrophilic polymeric group can be substituted with one to about six alkyl, fatty acid or fatty acid ester groups. Hydrophilic polymers that are substituted with a fatty acid or fatty acid ester group can be prepared by employing suitable methods. For example, a polymer comprising an amine group can be coupled to a carboxylate of the fatty acid or fatty acid ester, and an activated carboxylate (e.g., activated with N,N-carbonyl diimidazole) on a fatty acid or fatty acid ester can be coupled to a hydroxyl group on a polymer.

Fatty acids and fatty acid esters suitable for modifying Ig derived proteins of the invention can be saturated or can contain one or more units of unsaturation. Fatty acids that are suitable for modifying Ig derived proteins of the invention include, for example, n-dodecanoate (C₁₂, laurate), n-tetradecanoate (C₁₄, myristate), n-octadecanoate (C₁₈, stearate), n-eicosanoate (C₂₀, arachidate), n-docosanoate (C₂₂, behenate), n-triacontanoate (C₃₀), n-tetracontanoate (C₄₀), cis-Δ9-octadecanoate (C₁₈, oleate), all cis-Δ5,8,11,14-eicosatetraenoate (C₂₀, arachidonate), octanedioic acid, tetradecanedioic acid, octadecanedioic acid, docosanedioic acid, and the like. Suitable fatty acid esters include mono-esters of dicarboxylic acids that comprise a linear or branched lower alkyl group. The lower alkyl group can comprise from one to about twelve, preferably one to about six, carbon atoms.

The modified human Ig derived proteins and antigen-binding fragments can be prepared using suitable methods, such as by reaction with one or more modifying agents. A “modifying agent” as the term is used herein, refers to a suitable organic group (e.g., hydrophilic polymer, a fatty acid, a fatty acid ester) that comprises an activating group. An “activating group” is a chemical moiety or functional group that can, under appropriate conditions, react with a second chemical group thereby forming a covalent bond between the modifying agent and the second chemical group. For example, amine-reactive activating groups include electrophilic groups such as tosylate, mesylate, halo (chloro, bromo, fluoro, iodo), N-hydroxysuccinimidyl esters (NHS), and the like. Activating groups that can react with thiols include, for example, maleimide, iodoacetyl, acrylolyl, pyridyl disulfides, 5-thiol-2-nitrobenzoic acid thiol (TNB-thiol), and the like. An aldehyde functional group can be coupled to amine- or hydrazide-containing molecules, and an azide group can react with a trivalent phosphorous group to form phosphoramidate or phosphorimide linkages. Suitable methods to introduce activating groups into molecules are known in the art (see for example, Hermanson, G. T., Bioconjugate Techniques, Academic Press: San Diego, Calif. (1996)). An activating group can be bonded directly to the organic group (e.g., hydrophilic polymer, fatty acid, fatty acid ester), or through a linker moiety, for example a divalent C₁-C₁₂ group wherein one or more carbon atoms can be replaced by a heteroatom such as oxygen, nitrogen or sulfur. Suitable linker moieties include, for example, tetraethylene glycol, —(CH₂)₃—, —NH—(CH₂)₆—NH—, —(CH₂)₂—NH— and —CH₂—O—CH₂—CH₂—O—CH₂—CH₂—O—CH—NH—. Modifying agents that comprise a linker moiety can be produced, for example, by reacting a mono-Boc-alkyldiamine (e.g., mono-Boc-ethylenediamine, mono-Boc-diaminohexane) with a fatty acid in the presence of 1-ethyl-3-(3-dimethylaminopropyl) carbodiimide (EDC) to form an amide bond between the free amine and the fatty acid carboxylate. The Boc protecting group can be removed from the product by treatment with trifluoroacetic acid (TFA) to expose a primary amine that can be coupled to another carboxylate as described, or can be reacted with maleic anhydride and the resulting product cyclized to produce an activated maleimido derivative of the fatty acid. (See, for example, Thompson, et al., WO 92/16221 the entire teachings of which are incorporated herein by reference.)

The modified Ig derived proteins of the invention can be produced by reacting a human Ig derived protein or antigen-binding fragment with a modifying agent. For example, the organic moieties can be bonded to the Ig derived protein in a non-site specific manner by employing an amine-reactive modifying agent, for example, an NHS ester of PEG. Modified human Ig derived proteins or antigen-binding fragments can also be prepared by reducing disulfide bonds (e.g., intra-chain disulfide bonds) of an Ig derived protein or antigen-binding fragment. The reduced Ig derived protein or antigen-binding fragment can then be reacted with a thiol-reactive modifying agent to produce the modified Ig derived protein of the invention. Modified human Ig derived proteins and antigen-binding fragments comprising an organic moiety that is bonded to specific sites of an Ig derived protein or specified portion or variant of the present invention can be prepared using suitable methods, such as reverse proteolysis (Fisch et al., Bioconjugate Chem., 3:147-153 (1992); Werlen et al., Bioconjugate Chem., 5:411-417 (1994); Kumaran et al., Protein Sci. 6(10):2233-2241 (1997); Itoh et al., Bioorg. Chem., 24(1): 59-68 (1996); Capellas et al., Biotechnol. Bioeng., 56(4):456-463 (1997)), and the methods described in Hermanson, G. T., Bioconjugate Techniques, Academic Press: San Diego, Calif. (1996).

COPD-Related IG Derived Protein or Specified Portion or Variant Compositions

The present invention also provides at least one COPD-related Ig derived protein or specified portion or variant composition comprising at least one, at least two, at least three, at least four, at least five, at least six or more COPD-related Ig derived proteins or specified portions or variants thereof, as described herein and/or as known in the art that are provided in a non-naturally occurring composition, mixture or form. Such compositions comprise non-naturally occurring compositions comprising at least one or two full length, C- and/or N-terminally deleted variants, domains, fragments, or specified variants, of the COPD-related Ig derived protein amino acid sequence selected from the group consisting of 1-50% of the contiguous amino acids of SEQ ID NO:13, 14, 15, 16, 17 or specified fragments, domains or variants thereof. Such composition percentages are by weight, volume, concentration, molarity, or molality as liquid or dry solutions, mixtures, suspension, emulsions or colloids, as known in the art or as described herein.

COPD-related Ig derived protein or specified portion or variant compositions of the present invention can further comprise at least one of any suitable auxiliary, such as, but not limited to, diluent, binder, stabilizer, buffers, salts, lipophilic solvents, preservative, adjuvant or the like. Pharmaceutically acceptable auxiliaries are preferred. Non-limiting examples of, and methods of preparing such sterile solutions are well known in the art, such as, but limited to, Gennaro, Ed., Remington 's Pharmaceutical Sciences, 18^{th} Edition, Mack Publishing Co. (Easton, Pa.) 1990. Pharmaceutically acceptable carriers can be routinely selected that are suitable for the mode of administration, solubility and/or stability of the COPD-related composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/Ig derived protein or specified portion or variant components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like. One preferred amino acid is glycine.

Carbohydrate excipients suitable for use in the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present invention are mannitol, trehalose, and raffinose.

COPD-related compositions can also include a buffer or a pH adjusting agent; typically, the buffer is a salt prepared from an organic acid or base. Representative buffers include organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. Preferred buffers for use in the present compositions are organic acid salts such as citrate.

Additionally, the COPD-related Ig derived protein or specified portion or variant compositions of the invention can include polymeric excipients/additives such as polyvinylpyrrolidones, ficolls (a polymeric sugar), dextrates (e.g., cyclodextrins, such as 2-hydroxypropyl-β-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the COPD-related compositions according to the invention are known in the art, e.g., as listed in “Remington: The Science & Practice of Pharmacy”, 19^{th} ed., Williams & Williams, (1995), and in the “Physician's Desk Reference”, 52^{nd} ed., Medical Economics, Montvale, N.J. (1998), the disclosures of which are entirely incorporated herein by reference. Preferrred carrier or excipient materials are carbohydrates (e.g., saccharides and alditols) and buffers (e.g., citrate) or polymeric agents.

Formulations

As noted above, the invention provides for stable formulations, which is preferably a phosphate buffer with saline or a chosen salt, as well as preserved solutions and formulations containing a preservative as well as multi-use preserved formulations suitable for pharmaceutical or veterinary use, comprising at least one COPD-related Ig derived protein or specified portion or variant in a pharmaceutically acceptable formulation. Preserved formulations contain at least one known preservative or optionally selected from the group consisting of at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4., 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3. 0.4, 0.5, 0.9, 1.0%), 0.1-3% benzyl alcohol (e.g., 0.5, 0.9, 1.1., 1.5, 1.9, 2.0, 2.5%), 0.001-0.5% thimerosal (e.g., 0.005, 0.01), 0.001-2.0% phenol (e.g., 0.05, 0.25, 0.28, 0.5, 0.9, 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, 1.0%), and the like.

As noted above, the invention provides an article of manufacture, comprising packaging material and at least one vial comprising a solution of at least one COPD-related Ig derived protein or specified portion or variant with the prescribed buffers and/or preservatives, optionally in an aqueous diluent, wherein said packaging material comprises a label that indicates that such solution can be held over a period of 1, 2, 3, 4, 5, 6, 9, 12, 18, 20, 24, 30, 36, 40, 48, 54, 60, 66, 72 hours or greater. The invention further comprises an article of manufacture, comprising packaging material, a first vial comprising lyophilized at least one COPD-related Ig derived protein or specified portion or variant, and a second vial comprising an aqueous diluent of prescribed buffer or preservative, wherein said packaging material comprises a label that instructs a patient to reconstitute the at least one COPD-related Ig derived protein or specified portion or variant in the aqueous diluent to form a solution that can be held over a period of twenty-four hours or greater.

The at least one COPD-relatedIg derived protein or specified portion or variant used in accordance with the present invention can be produced by recombinant means, including from mammalian cell or transgenic preparations, or can be purified from other biological sources, as described herein or as known in the art.

The range of at least one COPD-related Ig derived protein or specified portion or variant in the product of the present invention includes amounts yielding upon reconstitution, if in a wet/dry system, concentrations from about 1.0 μg/ml to about 1000 mg/ml, although lower and higher concentrations are operable and are dependent on the intended delivery vehicle, e.g., solution formulations will differ from transdermal patch, pulmonary, transmucosal, or osmotic or micro pump methods.

Preferably, the aqueous diluent optionally further comprises a pharmaceutically acceptable preservative. Preferred preservatives include those selected from the group consisting of phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4 to about pH 10, and preferred ranges from about pH 5 to about pH 9, and a most preferred range of about 6.0 to about 8.0. Preferably the formulations of the present invention have pH between about 6.8 and about 7.8. Preferred buffers include phosphate buffers, most preferably sodium phosphate, particularly phosphate buffered saline (PBS).

Other additives, such as a pharmaceutically acceptable solubilizers like Tween 20 (polyoxyethylene (20) sorbitan monolaurate), Tween 40 (polyoxyethylene (20) sorbitan monopalmitate), Tween 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

The formulations of the present invention can be prepared by a process which comprises mixing at least one COPD-related Ig derived protein or specified portion or variant and a preservative selected from the group consisting of phenol, m-cresol, p-cresol, o-cresol, chlorocresol, benzyl alcohol, alkylparaben, (methyl, ethyl, propyl, butyl and the like), benzalkonium chloride, benzethonium chloride, sodium dehydroacetate and thimerosal or mixtures thereof in an aqueous diluent. Mixing the at least one COPD-related Ig derived protein or specified portion or variant and preservative in an aqueous diluent is carried out using conventional dissolution and mixing procedures. To prepare a suitable formulation, for example, a measured amount of at least one COPD-related Ig derived protein or specified portion or variant in buffered solution is combined with the desired preservative in a buffered solution in quantities sufficient to provide the protein and preservative at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

The claimed formulations can be provided to patients as clear solutions or as dual vials comprising a vial of lyophilized at least one COPD-related Ig derived protein or specified portion or variant that is reconstituted with a second vial containing water, a preservative and/or excipients, preferably a phosphate buffer and/or saline and a chosen salt, in an aqueous diluent. Either a single solution vial or dual vial requiring reconstitution can be reused multiple times and can suffice for a single or multiple cycles of patient treatment and thus can provide a more convenient treatment regimen than currently available.

The present claimed articles of manufacture are useful for administration over a period of immediately to twenty-four hours or greater. Accordingly, the presently claimed articles of manufacture offer significant advantages to the patient. Formulations of the invention can optionally be safely stored at temperatures of from about 2 to about 40° C. and retain the biologically activity of the protein for extended periods of time, thus, allowing a package label indicating that the solution can be held and/or used over a period of 6, 12, 18, 24, 36, 48, 72, or 96 hours or greater. If preserved diluent is used, such label can include use up to 11-12 months, one-half, one and a half, and/or two years.

The solutions of at least one COPD-related Ig derived protein or specified portion or variant in the invention can be prepared by a process that comprises mixing at least one Ig derived protein or specified portion or variant in an aqueous diluent. Mixing is carried out using conventional dissolution and mixing procedures. To prepare a suitable diluent, for example, a measured amount of at least one Ig derived protein or specified portion or variant in water or buffer is combined in quantities sufficient to provide the protein and optionally a preservative or buffer at the desired concentrations. Variations of this process would be recognized by one of ordinary skill in the art. For example, the order the components are added, whether additional additives are used, the temperature and pH at which the formulation is prepared, are all factors that may be optimized for the concentration and means of administration used.

The claimed products can be provided to patients as clear solutions or as dual vials comprising a vial of lyophilized at least one COPD-related Ig derived protein or specified portion or variant that is reconstituted with a second vial containing the aqueous diluent. Either a single solution vial or dual vial requiring reconstitution can be reused multiple times and can suffice for a single or multiple cycles of patient treatment and thus provides a more convenient treatment regimen than currently available.

The claimed products can be provided indirectly to patients by providing to pharmacies, clinics, or other such institutions and facilities, clear solutions or dual vials comprising a vial of lyophilized at least one COPD-related Ig derived protein or specified portion or variant that is reconstituted with a second vial containing the aqueous diluent. The clear solution in this case can be up to one liter or even larger in size, providing a large reservoir from which smaller portions of the at least one Ig derived protein or specified portion or variant solution can be retrieved one or multiple times for transfer into smaller vials and provided by the pharmacy or clinic to their customers and/or patients.

Recognized devices comprising these single vial systems include those pen-injector devices for delivery of a solution such as BD Pens, BD Autojector®, Humaject®, NovoPen®, B-D®Pen, AutoPen®, and OptiPen®, GenotropinPen®, Genotronorm Pen®, Humatro Pen®, Reco-Pen®, Roferon Pen®, Biojector®, iject®, J-tip Needle-Free Injector®, Intraject®, Medi-Ject®, e.g., as made or developed by Becton Dickensen (Franklin Lakes, N.J., www.bectondickenson.com), Disetronic (Burgdorf, Switzerland, www.disetronic.com; Bioject, Portland, Oreg. (www.bioject.com); National Medical Products, Weston Medical (Peterborough, UK, www.weston-medical.com), Medi-Ject Corp (Minneapolis, Minn., www.mediject.com). Recognized devices comprising a dual vial system include those pen-injector systems for reconstituting a lyophilized drug in a cartridge for delivery of the reconstituted solution such as the HumatroPen®.

The products presently claimed include packaging material. The packaging material provides, in addition to the information required by the regulatory agencies, the conditions under which the product can be used. The packaging material of the present invention provides instructions to the patient to reconstitute the at least one COPD-related Ig derived protein or specified portion or variant in the aqueous diluent to form a solution and to use the solution over a period of 2-24 hours or greater for the two vial, wet/dry, product. For the single vial, solution product, the label indicates that such solution can be used over a period of 2-24 hours or greater. The presently claimed products are useful for human pharmaceutical product use.

The formulations of the present invention can be prepared by a process that comprises mixing at least one COPD-related Ig derived protein or specified portion or variant and a selected buffer, preferably a phosphate buffer containing saline or a chosen salt. Mixing the at least one Ig derived protein or specified portion or variant and buffer in an aqueous diluent is carried out using conventional dissolution and mixing procedures. To prepare a suitable formulation, for example, a measured amount of at least one Ig derived protein or specified portion or variant in water or buffer is combined with the desired buffering agent in water in quantities sufficient to provide the protein and buffer at the desired concentrations. Variations of this process would be recognized by one of ordinary skill in the art. For example, the order the components are added, whether additional additives are used, the temperature and pH at which the formulation is prepared, are all factors that can be optimized for the concentration and means of administration used.

The claimed stable or preserved formulations can be provided to patients as clear solutions or as dual vials comprising a vial of lyophilized at least one COPD-related Ig derived protein or specified portion or variant that is reconstituted with a second vial containing a preservative or buffer and excipients in an aqueous diluent. Either a single solution vial or dual vial requiring reconstitution can be reused multiple times and can suffice for a single or multiple cycles of patient treatment and thus provides a more convenient treatment regimen than currently available.

At least one COPD-related Ig derived protein or specified portion or variant in either the stable or preserved formulations or solutions described herein, can be administered to a patient in accordance with the present invention via a variety of delivery methods including SC or IM injection; transdermal, pulmonary, transmucosal, implant, osmotic pump, cartridge, micro pump, or other means appreciated by the skilled artisan, as well-known in the art.

Therapeutic Applications

The present invention also provides a method for modulating or treating COPD related conditions, in a cell, tissue, organ, animal, or patient including, but not limited to, at least one of COPD, asthma, emphysema, chronic bronchitis or COPD associated immune related disease, cardiovascular disease, infectious, malignant and/or neurologic disease. Such a method can optionally comprise administering an effective amount of at least one composition or pharmaceutical composition comprising at least one COPD-related Ig derived protein or specified portion or variant to a cell, tissue, organ, animal or patient in need of such modulation, treatment or therapy.

The present invention also provides a method for modulating or treating at least one COPD associated immune related disease, in a cell, tissue, organ, animal, or patient including, but not limited to, at least one of rheumatoid arthritis, juvenile rheumatoid arthritis, systemic onset juvenile rheumatoid arthritis, psoriatic arthritis, ankylosing spondilitis, gastric ulcer, seronegative arthropathies, osteoarthritis, inflammatory bowel disease, ulcerative colitis, systemic lupus erythematosis, antiphospholipid syndrome, iridocyclitis/uveitis/optic neuritis, idiopathic pulmonary fibrosis, systemic vasculitis/wegener's granulomatosis, sarcoidosis, orchitis/vasectomy reversal procedures, allergic/atopic diseases, asthma, allergic rhinitis, eczema, allergic contact dermatitis, allergic conjunctivitis, hypersensitivity pneumonitis, transplants, organ transplant rejection, graft-versus-host disease, systemic inflammatory response syndrome, sepsis syndrome, gram positive sepsis, gram negative sepsis, culture negative sepsis, fungal sepsis, neutropenic fever, urosepsis, meningococcemia, trauma/hemorrhage, bums, ionizing radiation exposure, acute pancreatitis, adult respiratory distress syndrome, rheumatoid arthritis, alcohol-induced hepatitis, chronic inflammatory pathologies, sarcoidosis, Crohn's pathology, sickle cell anemia, diabetes, nephrosis, atopic diseases, hypersensitity reactions, allergic rhinitis, hay fever, perennial rhinitis, conjunctivitis, asthma, urticaria, systemic anaphalaxis, dermatitis, pernicious anemia, hemolytic disesease, thrombocytopenia, graft rejection of any organ or tissue, kidney translplant rejection, heart transplant rejection, liver transplant rejection, pancreas transplant rejection, lung transplant rejection, bone marrow transplant (BMT) rejection, skin allograft rejection, cartilage transplant rejection, bone graft rejection, small bowel transplant rejection, fetal thymus implant rejection, parathyroid transplant rejection, xenograft rejection of any organ or tissue, allograft rejection, anti-receptor hypersensitivity reactions, Graves disease, Raynoud's disease, type B insulin-resistant diabetes, asthma, myasthenia gravis, Ig derived protein-meditated cytotoxicity, type III hypersensitivity reactions, systemic lupus erythematosus, POEMS syndrome (polyneuropathy, organomegaly, endocrinopathy, monoclonal gammopathy, and skin changes syndrome), polyneuropathy, organomegaly, endocrinopathy, monoclonal gammopathy, skin changes syndrome, antiphospholipid syndrome, pemphigus, scleroderma, mixed connective tissue disease, idiopathic Addison's disease, diabetes mellitus, chronic active hepatitis, primary billiary cirrhosis, vitiligo, vasculitis, post-MI cardiotomy syndrome, type IV hypersensitivity, contact dermatitis, hypersensitivity pneumonitis, allograft rejection, granulomas due to intracellular organisms, drug sensitivity, metabolic/idiopathic, Wilson's disease, hemachromatosis, alpha-1-antitrypsin deficiency, diabetes, hashimoto's thyroiditis, osteoporosis, hypothalamic-pituitary-adrenal axis evaluation, primary biliary cirrhosis, thyroiditis, encephalomyelitis, cachexia, cystic fibrosis, neonatal chronic lung disease, chronic obstructive pulmonary disease (COPD), familial hematophagocytic lymphohistiocytosis, dermatologic conditions, psoriasis, alopecia, nephrotic syndrome, nephritis, glomerular nephritis, acute renal failure, hemodialysis, uremia, toxicity, preeclampsia, okt3 therapy, anti-cd3 therapy, cytokine therapy, chemotherapy, radiation therapy (e.g., including but not limited to asthenia, anemia, cachexia, and the like), chronic salicylate intoxication, and the like. See, e.g., the Merck Manual, 12th-17th Editions, Merck & Company, Rahway, N.J. (1972, 1977, 1982, 1987, 1992, 1999), Pharmacotherapy Handbook, Wells et al., eds., Second Edition, Appleton and Lange, Stamford, Conn. (1998, 2001), each entirely incorporated by reference.

The present invention also provides a method for modulating or treating at least one COPD associated cardiovascular disease in a cell, tissue, organ, animal, or patient, including, but not limited to, at least one of cardiac stun syndrome, myocardial infarction, congestive heart failure, stroke, ischemic stroke, hemorrhage, arteriosclerosis, atherosclerosis, diabetic ateriosclerotic disease, hypertension, arterial hypertension, renovascular hypertension, syncope, shock, syphilis of the cardiovascular system, heart failure, cor pulmonale, primary pulmonary hypertension, cardiac arrhythmias, atrial ectopic beats, atrial flutter, atrial fibrillation (sustained or paroxysmal), post perfusion syndrome, cardiopulmonary bypass inflammation response, chaotic or multifocal atrial tachycardia, regular narrow QRS tachycardia, specific arrythmias, ventricular fibrillation, His bundle arrythmias, atrioventricular block, bundle branch block, myocardial ischemic disorders, coronary artery disease, angina pectoris, myocardial infarction, cardiomyopathy, dilated congestive cardiomyopathy, restrictive cardiomyopathy, valvular heart diseases, endocarditis, pericardial disease, cardiac tumors, aordic and peripheral aneuryisms, aortic dissection, inflammation of the aorta, occulsion of the abdominal aorta and its branches, peripheral vascular disorders, occulsive arterial disorders, peripheral atherlosclerotic disease, thromboangitis obliterans, functional peripheral arterial disorders, Raynaud's phenomenon and disease, acrocyanosis, erythromelalgia, venous diseases, venous thrombosis, varicose veins, arteriovenous fistula, lymphederma, lipedema, unstable angina, reperfusion injury, post pump syndrome, ischemia-reperfusion injury, and the like. Such a method can optionally comprise administering an effective amount of a composition or pharmaceutical composition comprising at least one COPD-related Ig derived protein or specified portion or variant to a cell, tissue, organ, animal or patient in need of such modulation, treatment or therapy.

The present invention also provides a method for modulating or treating at least one COPD associated infectious disease in a cell, tissue, organ, animal or patient, including, but not limited to, at least one of: acute or chronic bacterial infection, acute and chronic parasitic or infectious processes, including bacterial, viral and fungal infections, HIV infection/HIV neuropathy, meningitis, hepatitis (A,B or C, or the like), septic arthritis, peritonitis, pneumonia, epiglottitis, e. coli 0157:h7, hemolytic uremic syndrome/thrombolytic thrombocytopenic purpura, malaria, dengue hemorrhagic fever, leishmaniasis, leprosy, toxic shock syndrome, streptococcal myositis, gas gangrene, mycobacterium tuberculosis, mycobacterium avium intracellulare, pneumocystis carinii pneumonia, pelvic inflammatory disease, orchitis/epidydimitis, legionella, lyme disease, influenza a, epstein-barr virus, vital-associated hemaphagocytic syndrome, vital encephalitis/aseptic meningitis, and the like;

The present invention also provides a method for modulating or treating at least one COPD associated malignant disease in a cell, tissue, organ, animal or patient, including, but not limited to, at least one of: leukemia, acute leukemia, acute lymphoblastic leukemia (ALL), B-cell, T-cell or FAB ALL, acute myeloid leukemia (AML), chromic myelocytic leukemia (CML), chronic lymphocytic leukemia (CLL), hairy cell leukemia, myelodyplastic syndrome (MDS), a lymphoma, Hodgkin's disease, a malignamt lymphoma, non-hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumors, adenocarcinomas, sarcomas, malignant melanoma, and the like.

The present invention also provides a method for modulating or treating at least one COPD associated neurologic disease in a cell, tissue, organ, animal or patient, including, but not limited to, at least one of: neurodegenerative diseases, multiple sclerosis, migraine headache, AIDS dementia complex, demyelinating diseases, such as multiple sclerosis and acute transverse myelitis; extrapyramidal and cerebellar disorders' such as lesions of the corticospinal system; disorders of the basal ganglia or cerebellar disorders; hyperkinetic movement disorders such as Huntington's Chorea and senile chorea; drug-induced movement disorders, such as those induced by drugs which block CNS dopamine receptors; hypokinetic movement disorders, such as Parkinson's disease; Progressive supranucleo Palsy; structural lesions of the cerebellum; spinocerebellar degenerations, such as spinal ataxia, Friedreich's ataxia, cerebellar cortical degenerations, multiple systems degenerations (Mencel, Dejerine-Thomas, Shi-Drager, and Machado-Joseph); systemic disorders (Refsum's disease, abetalipoprotemia, ataxia, telangiectasia, and mitochondrial multi.system disorder); demyelinating core disorders, such as multiple sclerosis, acute transverse myelitis; and disorders of the motor unit such as neurogenic muscular atrophies (anterior horn cell degeneration, such as amyotrophic lateral sclerosis, infantile spinal muscular atrophy and juvenile spinal muscular atrophy); Alzheimer's disease; Down's Syndrome in middle age; Diffuse Lewy body disease; Senile Dementia of Lewy body type; Wernicke-Korsakoff syndrome; chronic alcoholism; Creutzfeldt-Jakob disease; Subacute sclerosing panencephalitis, Hallerrorden-Spatz disease; and Dementia pugilistica, and the like. Such a method can optionally comprise administering an effective amount of a composition or pharmaceutical composition comprising at least one TNF antibody or specified portion or variant to a cell, tissue, organ, animal or patient in need of such modulation, treatment or therapy. See, e.g., the Merck Manual, 16^{th} Edition, Merck & Company, Rahway, N.J. (1992)

Any method of the present invention can comprise administering an effective amount of a composition or pharmaceutical composition comprising at least one COPD-related Ig derived protein or specified portion or variant to a cell, tissue, organ, animal or patient in need of such modulation, treatment or therapy. Such a method can optionally further comprise co-administration or combination therapy for treating such immune diseases, wherein the administering of said at least one COPD-related Ig derived protein, specified portion or variant thereof, further comprises administering, before concurrently, and/or after, at least one selected from at least one TNF antagonist which is also a COPD-related Ig derived protein of the present invention (e.g., but not limited to a TNF Ig derived protein or fragment, a soluble TNF receptor or fragment, fusion proteins thereof, or a small molecule TNF antagonist), an antirheumatic, a muscle relaxant, a narcotic, a non-steroid anti-inflammatory drug (NSAID), an analgesic, an anesthetic, a sedative, a local anethetic, a neuromuscular blocker, an antimicrobial (e.g., aminoglycoside, an antifungal, an antiparasitic, an antiviral, a carbapenem, cephalosporin, a flurorquinolone, a macrolide, a penicillin, a sulfonamide, a tetracycline, another antimicrobial), an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin (e.g., epoetin alpha), a filgrastim (e.g., G-CSF, Neupogen), a sargramostim (GM-CSF, Leukine), an immunization, an immunoglobulin, an immunosuppressive (e.g., basiliximab, cyclosporine, daclizumab), a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha (Pulmozyme), a cytokine or a cytokine antagonistm. Suitable dosages are well known in the art. See, e.g., Wells et al., eds., Pharmacotherapy Handbook, 2^{nd} Edition, Appleton and Lange, Stamford, CT (2000); PDR Pharmacopoeia, Tarascon Pocket Pharmacopoeia 2000, Deluxe Edition, Tarascon Publishing, Loma Linda, Calif. (2000), each of which references are entirely incorporated herein by reference.

TNF antagonists suitable for compositions, combination therapy, co-administration, devices and/or methods of the present invention (further comprising at least one anti body, specified portion and variant thereof, of the present invention), include, but are not limited to, anti-TNF Ig derived proteins, antigen-binding fragments thereof, and receptor molecules which bind specifically to TNF; compounds which prevent and/or inhibit TNF synthesis, TNF release or its action on target cells, such as thalidomide, tenidap, phosphodiesterase inhibitors (e.g, pentoxifylline and rolipram), A2b adenosine receptor agonists and A2b adenosine receptor enhancers; compounds which prevent and/or inhibit TNF receptor signalling, such as mitogen activated protein (MAP) kinase inhibitors; compounds which block and/or inhibit membrane TNF cleavage, such as metalloproteinase inhibitors; compounds which block and/or inhibit TNF activity, such as angiotensin converting enzyme (ACE) inhibitors (e.g., captopril); and compounds which block and/or inhibit TNF production and/or synthesis, such as MAP kinase inhibitors.

As used herein, a “tumor necrosis factor Ig derived protein,” “TNF Ig derived protein,” “TNFα Ig derived protein,” or fragment and the like decreases, blocks, inhibits, abrogates or interferes with TNFα activity in vitro, in situ and/or preferably in vivo. For example, a suitable TNF human Ig derived protein of the present invention can bind TNFα and includes anti-TNF Ig derived proteins, antigen-binding fragments thereof, and specified mutants or domains thereof that bind specifically to TNFα. A suitable TNF anttibody or fragment can also decrease block, abrogate, interfere, prevent and/or inhibit TNF RNA, DNA or protein synthesis, TNF release, TNF receptor signaling, membrane TNF cleavage, TNF activity, TNF production and/or synthesis.

Chimeric Ig derived protein cA2 consists of the antigen binding variable region of the high-affinity neutralizing mouse anti-human TNFα IgG1 Ig derived protein, designated A2, and the constant regions of a human IgG1, kappa immunoglobulin. The human IgG1 Fc region improves allogeneic Ig derived protein effector function, increases the circulating serum half-life and decreases the immunogenicity of the Ig derived protein. The avidity and epitope specificity of the chimeric Ig derived protein cA2 is derived from the variable region of the murine Ig derived protein A2. In a particular embodiment, a preferred source for nucleic acids encoding the variable region of the murine Ig derived protein A2 is the A2 hybridoma cell line.

Chimeric A2 (cA2) neutralizes the cytotoxic effect of both natural and recombinant human TNF in a dose dependent manner. From binding assays of chimeric Ig derived protein cA2 and recombinant human TNF, the affinity constant of chimeric Ig derived protein cA2 was calculated to be 1.04×10¹⁰M⁻¹. Preferred methods for determining monoclonal Ig derived protein specificity and affinity by competitive inhibition can be found in Harlow, et al., Ig derived proteins: A Laboratory Manual, Cold Spring Harbor Laboratory Press, Cold Spring Harbor, N.Y., 1988; Colligan et al., eds., Current Protocols in Immunology, Greene Publishing Assoc. and Wiley Interscience, New York, (1992-2001); Kozbor et al., Immunol. Today, 4:72-79 (1983); Ausubel et al., eds. Current Protocols in Molecular Biology, Wiley Interscience, New York (1987-2001); and Muller, Meth. Enzymol., 92:589-601 (1983), which references are entirely incorporated herein by reference.

In a particular embodiment, murine monoclonal Ig derived protein A2 is produced by a cell line designated c 134A. Chimeric Ig derived protein cA2 is produced by a cell line designated c168A.

Additional examples of monoclonal anti-TNF Ig derived proteins that can be used in the present invention are described in the art (see, e.g., U.S. Pat. No. 5,231,024; Möller, A. et al., Cytokine 2(3): 162-169 (1990); U.S. application Ser. No. 07/943,852 (filed Sep. 11, 1992); Rathjen et al., International Publication No. WO 91/02078 (published Feb. 21, 1991); Rubin et al., EPO Patent Publication No. 0 218 868 (published Apr. 22, 1987); Yone et al., EPO Patent Publication No. 0 288 088 (Oct. 26, 1988); Liang, et al., Biochem. Biophys. Res. Comm. 137:847-854 (1986); Meager, et al., Hybridoma 6:305-311 (1987); Fendly et al., Hybridoma 6:359-369 (1987); Bringman, et al., Hybridoma 6:489-507 (1987); and Hirai, et al., J. Immunol. Meth. 96:57-62 (1987), which references are entirely incorporated herein by reference).

TNF Receptor Molecules

Preferred TNF receptor molecules useful in the present invention are those that bind TNF with high affinity (see, e.g., Feldmann et al., International Publication No. WO 92/07076 (published Apr. 30, 1992); Schall et al., Cell 61:361-370 (1990); and Loetscher et al., Cell 61:351-359 (1990), which references are entirely incorporated herein by reference) and optionally possess low immunogenicity. In particular, the 55 kDa (p55 TNF-R) and the 75 kDa (p75 TNF-R) TNF cell surface receptors are useful in the present invention. Truncated forms of these receptors, comprising the extracellular domains (ECD) of the receptors or functional portions thereof (see, e.g., Corcoran et al., Eur. J. Biochem. 223:831-840 (1994)), are also useful in the present invention. Truncated forms of the TNF receptors, comprising the ECD, have been detected in urine and serum as 30 kDa and 40 kDa TNF inhibitory binding proteins (Engelmann, H. et al., J. Biol. Chem. 265:1531-1536 (1990)). TNF receptor multimeric molecules and TNF immunoreceptor fusion molecules, and derivatives and fragments or portions thereof, are additional examples of TNF receptor molecules which are useful in the methods and compositions of the present invention. The TNF receptor molecules which can be used in the invention are characterized by their ability to treat patients for extended periods with good to excellent alleviation of symptoms and low toxicity. Low immunogenicity and/or high affinity, as well as other undefined properties, may contribute to the therapeutic results achieved.

TNF receptor multimeric molecules useful in the present invention comprise all or a functional portion of the ECD of two or more TNF receptors linked via one or more polypeptide linkers or other nonpeptide linkers, such as polyethylene glycol (PEG). The multimeric molecules can further comprise a signal peptide of a secreted protein to direct expression of the multimeric molecule. These multimeric molecules and methods for their production have been described in U.S. application Ser. No. 08/437,533 (filed May 9, 1995), the content of which is entirely incorporated herein by reference.

TNF immunoreceptor fusion molecules useful in the methods and compositions of the present invention comprise at least one portion of one or more immunoglobulin molecules and all or a functional portion of one or more TNF receptors. These immunoreceptor fusion molecules can be assembled as monomers, or hetero- or homo-multimers. The immunoreceptor fusion molecules can also be monovalent or multivalent. An example of such a TNF immunoreceptor fusion molecule is TNF receptor/IgG fusion protein. TNF immunoreceptor fusion molecules and methods for their production have been described in the art (Lesslauer et al., Eur. J. Immunol. 21:2883-2886 (1991); Ashkenazi et al., Proc. Natl. Acad. Sci. USA 88:10535-10539 (1991); Peppel et al., J. Exp. Med. 174:1483-1489 (1991); Kolls et al., Proc. Natl. Acad. Sci. USA 91:215-219 (1994); Butler et al., Cytokine 6(6):616-623 (1994); Baker et al., Eur. J. Immunol. 24:2040-2048 (1994); Beutler et al., U.S. Pat. No. 5,447,851; and U.S. application Ser. No. 08/442,133 (filed May 16, 1995), each of which references are entirely incorporated herein by reference). Methods for producing immunoreceptor fusion molecules can also be found in Capon et al., U.S. Pat. No. 5,116,964; Capon et al., U.S. Pat. No. 5,225,538; and Capon et al., Nature 337:525-531 (1989), which references are entirely incorporated herein by reference.

A functional equivalent, derivative, fragment or region of TNF receptor molecule refers to the portion of the TNF receptor molecule, or the portion of the TNF receptor molecule sequence which encodes TNF receptor molecule, that is of sufficient size and sequences to functionally resemble TNF receptor molecules that can be used in the present invention (e.g., bind TNF with high affinity and possess low immunogenicity). A functional equivalent of TNF receptor molecule also includes modified TNF receptor molecules that functionally resemble TNF receptor molecules that can be used in the present invention (e.g., bind TNF with high affinity and possess low immunogenicity). For example, a functional equivalent of TNF receptor molecule can contain a “SILENT” codon or one or more amino acid substitutions, deletions or additions (e.g., substitution of one acidic amino acid for another acidic amino acid; or substitution of one codon encoding the same or different hydrophobic amino acid for another codon encoding a hydrophobic amino acid). See Ausubel et al., Current Protocols in Molecular Biology, Greene Publishing Assoc. and Wiley-Interscience, New York (1987-2001).

Cytokines include any known cytokine. See, e.g., CopewithCytokines.com. Cytokine antagonists include, but are not limited to, any Ig derived protein, fragment or mimetic, any soluble receptor, fragment or mimetic, any small molecule antagonist, or any combination thereof.

Therapeutic Treatments.

Any method of the present invention can comprise a method for treating a COPD-related mediated disorder, comprising administering an effective amount of a composition or pharmaceutical composition comprising at least one COPD-related Ig derived protein or specified portion or variant to a cell, tissue, organ, animal or patient in need of such modulation, treatment or therapy.

Typically, treatment of pathologic conditions is effected by administering an effective amount or dosage of at least one COPD-related composition that total, on average, a range from at least about 0.01 to 500 milligrams of at least one COPD-relatedIg derived protein or specified portion or variant/kilogram of patient per dose, and preferably from at least about 0.1 to 100 milligrams Ig derived protein or specified portion or variant/kilogram of patient per single or multiple administration, depending upon the specific activity of contained in the composition. Alternatively, the effective serum concentration can comprise 0.1-5000 μg/ml serum concentration per single or multiple adminstration. Suitable dosages are known to medical practitioners and will, of course, depend upon the particular disease state, specific activity of the composition being administered, and the particular patient undergoing treatment. In some instances, to achieve the desired therapeutic amount, it can be necessary to provide for repeated administration, i.e., repeated individual administrations of a particular monitored or metered dose, where the individual administrations are repeated until the desired daily dose or effect is achieved.

Preferred doses can optionally include 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 mg/kg/administration, or any range, value or fraction thereof, or to achieve a serum concentration of 0.1, 0.5, 0.9, 1.0, 1.1, 1.2, 1.5, 1.9, 2.0, 2.5, 2.9, 3.0, 3.5, 3.9, 4.0, 4.5, 4.9, 5.0, 5.5, 5.9, 6.0, 6.5, 6.9, 7.0, 7.5, 7.9, 8.0, 8.5, 8.9, 9.0, 9.5, 9.9, 10, 10.5, 10.9, 11, 11.5, 11.9, 20, 12.5, 12.9, 13.0, 13.5, 13.9, 14.0, 14.5, 4.9, 5.0, 5.5., 5.9, 6.0, 6.5, 6.9, 7.0, 7.5, 7.9, 8.0, 8.5, 8.9, 9.0,9.5, 9.9, 10, 10.5, 10.9, 11, 11.5, 11.9, 12, 12.5, 12.9, 13.0, 13.5, 13.9, 14, 14.5, 15, 15.5, 15.9, 16, 16.5, 16.9, 17, 17.5, 17.9, 18, 18.5, 18.9, 19,19.5, 19.9, 20, 20.5, 20.9, 21, 22, 23, 24, 25, 26, 27, 28, 29, 30, 35, 40, 45, 50, 55, 60, 65, 70, 75, 80, 85, 90, 96, 100, 200, 300,400, 500, 600, 700, 800, 900, 1000, 1500, 2000, 2500, 3000, 3500, 4000, 4500, and/or 5000 μg/ml serum concentration per single or multiple administration, or any range, value or fraction thereof.

Alternatively, the dosage administered can vary depending upon known factors, such as the pharmacodynamic characteristics of the particular agent, and its mode and route of administration; age, health, and weight of the recipient; nature and extent of symptoms, kind of concurrent treatment, frequency of treatment, and the effect desired. Usually a dosage of active ingredient can be about 0.1 to 100 milligrams per kilogram of body weight. Ordinarily 0.1 to 50, and preferably 0.1 to 10 milligrams per kilogram per administration or in sustained release form is effective to obtain desired results.

As a non-limiting example, treatment of humans or animals can be provided as a one-time or periodic dosage of at least one Ig derived protein or specified portion or variant of the present invention 0.1 to 100 mg/kg, such as 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day, on at least one of day 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40, or alternatively or additionally, at least one of week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or 52, or alternatively or additionally, at least one of 1, 2, 3, 4, 5, 6, 7, 8, 9, 10, 11, 12, 13, 14, 15, 16, 17, 18, 19, or 20 years, or any combination thereof, using single, infusion or repeated doses.

Dosage forms (composition) suitable for internal administration generally contain from about 0.1 milligram to about 500 milligrams of active ingredient per unit or container. In these pharmaceutical compositions the active ingredient will ordinarily be present in an amount of about 0.5-99.999% by weight based on the total weight of the composition.

For parenteral administration, the Ig derived protein or specified portion or variant can be formulated as a solution, suspension, emulsion or lyophilized powder in association, or separately provided, with a pharmaceutically acceptable parenteral vehicle. Examples of such vehicles are water, saline, Ringer's solution, dextrose solution, and 1-10% human serum albumin. Liposomes and nonaqueous vehicles such as fixed oils may also be used. The vehicle or lyophilized powder may contain additives that maintain isotonicity (e.g., sodium chloride, mannitol) and chemical stability (e.g., buffers and preservatives). The formulation is sterilized by known or suitable techniques.

Suitable pharmaceutical carriers are described in the most recent edition of Remington's Pharmaceutical Sciences, A. Osol, a standard reference text in this field.

Alternative Administration

Many known and developed modes of can be used according to the present invention for administering pharmaceutically effective amounts of at least one COPD-related Ig derived protein or specified portion or variant according to the present invention. While pulmonary administration is used in the following description, other modes of administration can be used according to the present invention with suitable results.

COPD-related Ig derived proteins of the present invention can be delivered in a carrier, as a solution, emulsion, colloid, or suspension, or as a dry powder, using any of a variety of devices and methods suitable for administration by inhalation or other modes described here within or known in the art.

Parenteral Formulations and Administration

Formulations for parenteral administration can contain as common excipients sterile water or saline, polyalkylene glycols such as polyethylene glycol, oils of vegetable origin, hydrogenated naphthalenes and the like. Aqueous or oily suspensions for injection can be prepared by using an appropriate emulsifier or humidifier and a suspending agent, according to known methods. Agents for injection can be a non-toxic, non-orally administrable diluting agent such as aquous solution or a sterile injectable solution or suspension in a solvent. As the usable vehicle or solvent, water, Ringer's solution, isotonic saline, etc. are allowed; as an ordinary solvent, or suspending solvent, sterile involatile oil can be used. For these purposes, any kind of involatile oil and fatty acid can be used, including natural or synthetic or semisynthetic fatty oils or fatty acids; natural or synthetic or semisynthtetic mono- or di- or tri-glycerides. Parental administration is known in the art and includes, but is not limited to, conventional means of injections, a gas pressured needle-less injection device as described in U.S. Pat. No. 5,851,198, and a laser perforator device as described in U.S. Pat. No. 5,839,446 entirely incorporated herein by reference.

Alternative Delivery

The invention further relates to the administration of at least one COPD-related Ig derived protein or specified portion or variant by parenteral, subcutaneous, intramuscular, intravenous, bolus, vaginal, rectal, buccal, sublingual, intranasal, or transdermal means. Protein, Ig derived protein or specified portion or variant compositions can be prepared for use for parenteral (subcutaneous, intramuscular or intravenous) administration particularly in the form of liquid solutions or suspensions; for use in vaginal or rectal administration particularly in semisolid forms such as creams and suppositories; for buccal, or sublingual administration particularly in the form of tablets or capsules; or intranasally particularly in the form of powders, nasal drops or aerosols or certain agents; or transdermally particularly in the form of a gel, ointment, lotion, suspension or patch delivery system with chemical enhancers such as dimethyl sulfoxide to either modify the skin structure or to increase the drug concentration in the transdermal patch (Junginger, et al. In “Drug Permeation Enhancement”; Hsieh, D. S., Eds., pp. 59-90 (Marcel Dekker, Inc. New York 1994, entirely incorporated herein by reference), or with oxidizing agents that enable the application of formulations containing proteins and peptides onto the skin (WO 98/53847), or applications of electric fields to create transient transport pathways such as electroporation, or to increase the mobility of charged drugs through the skin such as iontophoresis, or application of ultrasound such as sonophoresis (U.S. Pat. Nos. 4,309,989 and 4,767,402) (the above publications and patents being entirely incorporated herein by reference).

Pulmonary/Nasal Administration

For pulmonary administration, preferably at least one COPD-related Ig derived protein or specified portion or variant composition is delivered in a particle size effective for reaching the lower airways of the lung or sinuses. According to the invention, at least one COPD-related Ig derived protein or specified portion or variant can be delivered by any of a variety of inhalation or nasal devices known in the art for administration of a therapeutic agent by inhalation. These devices capable of depositing aerosolized formulations in the sinus cavity or alveoli of a patient include metered dose inhalers, nebulizers, dry powder generators, sprayers, and the like. Other devices suitable for directing the pulmonary or nasal administration of Ig derived protein or specified portion or variants are also known in the art. All such devices can use of formulations suitable for the administration for the dispensing of Ig derived protein or specified portion or variant in an aerosol. Such aerosols can be comprised of either solutions (both aqueous and non aqueous) or solid particles. Metered dose inhalers like the Ventolin® metered dose inhaler, typically use a propellent gas and require actuation during inspiration (See, e.g., WO 94/16970, WO 98/35888). Dry powder inhalers like Turbuhaler™ (Astra), Rotahaler® (Glaxo), Diskus® (Glaxo), Spiros™ inhaler (Dura), devices marketed by Inhale Therapeutics, and the Spinhaler® powder inhaler (Fisons), use breath-actuation of a mixed powder (U.S. Pat. No. 4,668,218 Astra, EP 237507 Astra, WO 97/25086 Glaxo, WO 94/08552 Dura, U.S. Pat. No. 5,458,135 Inhale, WO 94/06498 Fisons, entirely incorporated herein by reference). Nebulizers like AERx™ Aradigm, the Ultravent® nebulizer (Mallinckrodt), and the Acorn II® nebulizer (Marquest Medical Products) (U.S. Pat. No. 5,404,871 Aradigm, WO 97/22376), the above references entirely incorporated herein by reference, produce aerosols from solutions, while metered dose inhalers, dry powder inhalers, etc. generate small particle aerosols. These specific examples of commercially available inhalation devices are intended to be a representative of specific devices suitable for the practice of this invention, and are not intended as limiting the scope of the invention. Preferably, a composition comprising at least one COPD-related Ig derived protein or specified portion or variant is delivered by a dry powder inhaler or a sprayer. There are a several desirable features of an inhalation device for administering at least one Ig derived protein or specified portion or variant of the present invention. For example, delivery by the inhalation device is advantageously reliable, reproducible, and accurate. The inhalation device can optionally deliver small dry particles, e.g. less than about 10 μm, preferably about 1-5 μm, for good respirability.

Administration of COPD-Related Ig Derived Protein or Specified Portion or Variant Compositions as a Spray

A spray including COPD-related Ig derived protein or specified portion or variant composition protein can be produced by forcing a suspension or solution of at least one COPD-related Ig derived protein or specified portion or variant through a nozzle under pressure. The nozzle size and configuration, the applied pressure, and the liquid feed rate can be chosen to achieve the desired output and particle size. An electrospray can be produced, for example, by an electric field in connection with a capillary or nozzle feed. Advantageously, particles of at least one COPD-related Ig derived protein or specified portion or variant composition protein delivered by a sprayer have a particle size less than about 10 μm, preferably in the range of about 1 μm to about 5 μm, and most preferably about 2 μm to about 3 μm.

Formulations of at least one COPD-related Ig derived protein or specified portion or variant composition protein suitable for use with a sprayer typically include Ig derived protein or specified portion or variant composition protein in an aqueous solution at a concentration of about 0.1 mg to about 100 mg of at least one COPD-related Ig derived protein or specified portion or variant composition protein per ml of solution or mg/gm, or any range or value therein, e.g., but not limited to, 1.0. 0.2., 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/ml or mg/gm. The formulation can include agents such as an excipient, a buffer, an isotonicity agent, a preservative, a surfactant, and, preferably, zinc. The formulation can also include an excipient or agent for stabilization of the Ig derived protein or specified portion or variant composition protein, such as a buffer, a reducing agent, a bulk protein, or a carbohydrate. Bulk proteins useful in formulating Ig derived protein or specified portion or variant composition proteins include albumin, protamine, or the like. Typical carbohydrates useful in formulating Ig derived protein or specified portion or variant composition proteins include sucrose, mannitol, lactose, trehalose, glucose, or the like. The Ig derived protein or specified portion or variant composition protein formulation can also include a surfactant, which can reduce or prevent surface-induced aggregation of the Ig derived protein or specified portion or variant composition protein caused by atomization of the solution in forming an aerosol. Various conventional surfactants can be employed, such as polyoxyethylene fatty acid esters and alcohols, and polyoxyethylene sorbitol fatty acid esters. Amounts will generally range between 0.001 and 14% by weight of the formulation. Especially preferred surfactants for purposes of this invention are polyoxyethylene sorbitan monooleate, polysorbate 80, polysorbate 20, or the like. Additional agents known in the art for formulation of a protein such as COPD-related Ig derived proteins, or specified portions or variants, can also be included in the formulation.

Administration of COPD-Related Ig Derived Protein or Specified Portion or Variant Compositions by a Nebulizer

Ig derived protein or specified portion or variant composition protein can be administered by a nebulizer, such as jet nebulizer or an ultrasonic nebulizer. Typically, in a jet nebulizer, a compressed air source is used to create a high-velocity air jet through an orifice. As the gas expands beyond the nozzle, a low-pressure region is created, which draws a solution of Ig derived protein or specified portion or variant composition protein through a capillary tube connected to a liquid reservoir. The liquid stream from the capillary tube is sheared into unstable filaments and droplets as it exits the tube, creating the aerosol. A range of configurations, flow rates, and baffle types can be employed to achieve the desired performance characteristics from a given jet nebulizer. In an ultrasonic nebulizer, high-frequency electrical energy is used to create vibrational, mechanical energy, typically employing a piezoelectric transducer. This energy is transmitted to the formulation of Ig derived protein or specified portion or variant composition protein either directly or through a coupling fluid, creating an aerosol including the Ig derived protein or specified portion or variant composition protein. Advantageously, particles of Ig derived protein or specified portion or variant composition protein delivered by a nebulizer have a particle size less than about 10 μm, preferably in the range of about 1 μm to about 5 μm, and most preferably about 2 μm to about 3 μm.

Formulations of at least one COPD-related Ig derived protein or specified portion or variant suitable for use with a nebulizer, either jet or ultrasonic, typically include a concentration of about 0.1 mg to about 100 mg of at least one COPD-related Ig derived protein or specified portion or variant protein per ml of solution. The formulation can include agents such as an excipient, a buffer, an isotonicity agent, a preservative, a surfactant, and, preferably, zinc. The formulation can also include an excipient or agent for stabilization of the at least one COPD-related Ig derived protein or specified portion or variant composition protein, such as a buffer, a reducing agent, a bulk protein, or a carbohydrate. Bulk proteins useful in formulating at least one COPD-related Ig derived protein or specified portion or variant composition proteins include albumin, protamine, or the like. Typical carbohydrates useful in formulating at least one COPD-related Ig derived protein or specified portion or variant include sucrose, mannitol, lactose, trehalose, glucose, or the like. The at least one COPD-related Ig derived protein or specified portion or variant formulation can also include a surfactant, which can reduce or prevent surface-induced aggregation of the at least one COPD-related Ig derived protein or specified portion or variant caused by atomization of the solution in forming an aerosol. Various conventional surfactants can be employed, such as polyoxyethylene fatty acid esters and alcohols, and polyoxyethylene sorbital fatty acid esters. Amounts will generally range between 0.001 and 4% by weight of the formulation. Especially preferred surfactants for purposes of this invention are polyoxyethylene sorbitan mono-oleate, polysorbate 80, polysorbate 20, or the like. Additional agents known in the art for formulation of a protein such as Ig derived protein or specified portion or variant protein can also be included in the formulation.

Administration of COPD-Related Ig Derived Protein or Specified Portion or Variant Compositions By A Metered Dose Inhaler

In a metered dose inhaler (MDI), a propellant, at least one COPD-related Ig derived protein or specified portion or variant, and any excipients or other additives are contained in a canister as a mixture including a liquefied compressed gas. Actuation of the metering valve releases the mixture as an aerosol, preferably containing particles in the size range of less than about 10 μm, preferably about 1 μm to about 5 μm, and most preferably about 2 μm to about 3 μm. The desired aerosol particle size can be obtained by employing a formulation of Ig derived protein or specified portion or variant composition protein produced by various methods known to those of skill in the art, including jet-milling, spray drying, critical point condensation, or the like. Preferred metered dose inhalers include those manufactured by 3M or Glaxo and employing a hydrofluorocarbon propellant.

Formulations of at least one COPD-related Ig derived protein or specified portion or variant for use with a metered-dose inhaler device will generally include a finely divided powder containing at least one COPD-related Ig derived protein or specified portion or variant as a suspension in a non-aqueous medium, for example, suspended in a propellant with the aid of a surfactant. The propellant can be any conventional material employed for this purpose, such as chlorofluorocarbon, a hydrochlorofluorocarbon, a hydrofluorocarbon, or a hydrocarbon, including trichlorofluoromethane, dichlorodifluoromethane, dichlorotetrafluoroethanol and 1,1,1,2-tetrafluoroethane, HFA-134a (hydrofluroalkane-134a), HFA-227 (hydrofluroalkane-227), or the like. Preferably the propellant is a hydrofluorocarbon. The surfactant can be chosen to stabilize the at least one COPD-related Ig derived protein or specified portion or variant as a suspension in the propellant, to protect the active agent against chemical degradation, and the like. Suitable surfactants include sorbitan trioleate, soya lecithin, oleic acid, or the like. In some cases solution aerosols are preferred using solvents such as ethanol. Additional agents known in the art for formulation of a protein such as protein can also be included in the formulation.

One of ordinary skill in the art will recognize that the methods of the current invention can be achieved by pulmonary administration of at least one COPD-related Ig derived protein or specified portion or variant compositions via devices not described herein.

Oral Formulations and Administration

Formulations for oral rely on the co-administration of adjuvants (e.g., resorcinols and nonionic surfactants such as polyoxyethylene oleyl ether and n-hexadecylpolyethylene ether) to increase artificially the permeability of the intestinal walls, as well as the co-administration of enzymatic inhibitors (e.g., pancreatic trypsin inhibitors, diisopropylfluorophosphate (DFF) and trasylol) to inhibit enzymatic degradation. The active constituent compound of the solid-type dosage form for oral administration can be mixed with at least one additive, including sucrose, lactose, cellulose, mannitol, trehalose, raffinose, maltitol, dextran, starches, agar, arginates, chitins, chitosans, pectins, gum tragacanth, gum arabic, gelatin, collagen, casein, albumin, synthetic or semisynthetic polymer, and glyceride. These dosage forms can also contain other type(s) of additives, e.g., inactive diluting agent, lubricant such as magnesium stearate, paraben, preserving agent such as sorbic acid, ascorbic acid, .alpha.-tocopherol, antioxidant such as cysteine, disintegrator, binder, thickener, buffering agent, sweetening agent, flavoring agent, perfuming agent, etc.

Tablets and pills can be further processed into enteric-coated preparations. The liquid preparations for oral administration include emulsion, syrup, elixir, suspension and solution preparations allowable for medical use. These preparations may contain inactive diluting agents ordinarily used in said field, e.g., water. Liposomes have also been described as drug delivery systems for insulin and heparin (U.S. Pat. No. 4,239,754). More recently, microspheres of artificial polymers of mixed amino acids (proteinoids) have been used to deliver pharmaceuticals (U.S. Pat. No. 4,925,673). Furthermore, carrier compounds described in U.S. Pat. No. 5,879,681 and U.S. Pat. No. 5,5,871,753 are used to deliver biologically active agents orally are known in the art.

Mucosal Formulations and Administration

For absorption through mucosal surfaces, compositions and methods of administering at least one COPD-related Ig derived protein or specified portion or variant include an emulsion comprising a plurality of submicron particles, a mucoadhesive macromolecule, a bioactive peptide, and an aqueous continuous phase, which promotes absorption through mucosal surfaces by achieving mucoadhesion of the emulsion particles (U.S. Pat. Nos. 5,514,670). Mucous surfaces suitable for application of the emulsions of the present invention can include corneal, conjunctival, buccal, sublingual, nasal, vaginal, pulmonary, stomachic, intestinal, and rectal routes of administration. Formulations for vaginal or rectal administration, e.g. suppositories, can contain as excipients, for example, polyalkyleneglycols, vaseline, cocoa butter, and the like. Formulations for intranasal administration can be solid and contain as excipients, for example, lactose or can be aqueous or oily solutions of nasal drops. For buccal administration excipients include sugars, calcium stearate, magnesium stearate, pregelinatined starch, and the like (U.S. Pat. Nos. 5,849,695).

Transdermal Formulations and Administration

For transdermal administration, the at least one COPD-related Ig derived protein or specified portion or variant is encapsulated in a delivery device such as a liposome or polymeric nanoparticles, microparticle, microcapsule, or microspheres (referred to collectively as microparticles unless otherwise stated). A number of suitable devices are known, including microparticles made of synthetic polymers such as polyhydroxy acids such as polylactic acid, polyglycolic acid and copolymers thereof, polyorthoesters, polyanhydrides, and polyphosphazenes, and natural polymers such as collagen, polyamino acids, albumin and other proteins, alginate and other polysaccharides, and combinations thereof (U.S. Pat. Nos. 5,814,599).

Prolonged Administration and Formulations

It can be sometimes desirable to deliver the compounds of the present invention to the subject over prolonged periods of time, for example, for periods of one week to one year from a single administration. Various slow release, depot or implant dosage forms can be utilized. For example, a dosage form can contain a pharmaceutically acceptable non-toxic salt of the compounds that has a low degree of solubility in body fluids, for example, (a) an acid addition salt with a polybasic acid such as phosphoric acid, sulfuric acid, citric acid, tartaric acid, tannic acid, pamoic acid, alginic acid, polyglutamic acid, naphthalene mono- or di-sulfonic acids, polygalacturonic acid, and the like; (b) a salt with a polyvalent metal cation such as zinc, calcium, bismuth, barium, magnesium, aluminum, copper, cobalt, nickel, cadmium and the like, or with an organic cation formed from e.g., N,N′-dibenzyl-ethylenediamine or ethylenediamine; or (c) combinations of (a) and (b) e.g. a zinc tannate salt. Additionally, the compounds of the present invention or, preferably, a relatively insoluble salt such as those just described, can be formulated in a gel, for example, an aluminum monostearate gel with, e.g. sesame oil, suitable for injection. Particularly preferred salts are zinc salts, zinc tannate salts, pamoate salts, and the like. Another type of slow release depot formulation for injection would contain the compound or salt dispersed for encapsulated in a slow degrading, non-toxic, non-antigenic polymer such as a polylactic acid/polyglycolic acid polymer for example as described in U.S. Pat. No. 3,773,919. The compounds or, preferably, relatively insoluble salts such as those described above can also be formulated in cholesterol matrix silastic pellets, particularly for use in animals. Additional slow release, depot or implant formulations, e.g. gas or liquid liposomes are known in the literature (U.S. Pat. Nos. 5,770,222 and “Sustained and Controlled Release Drug Delivery Systems”, J. R. Robinson ed., Marcel Dekker, Inc., N.Y., 1978).

Having generally described the invention, the same will be more readily understood by reference to the following examples, which are provided by way of illustration and are not intended as limiting.

### EXAMPLE 1

### Cloning and Expression of COPD-Related in Mammalian Cells

A typical mammalian expression vector contains at least one promoter element, which mediates the initiation of transcription of mRNA, the Ig derived protein or specified portion or variant coding sequence, and signals required for the termination of transcription and polyadenylation of the transcript. Additional elements include enhancers, Kozak sequences and intervening sequences flanked by donor and acceptor sites for RNA splicing. Highly efficient transcription can be achieved with the early and late promoters from SV40, the long terminal repeats (LTRS) from Retroviruses, e.g., RSV, HTLVI, HIVI and the early promoter of the cytomegalovirus (CMV). However, cellular elements can also be used (e.g., the human actin promoter). Suitable expression vectors for use in practicing the present invention include, for example, vectors such as pIRES1neo, pRetro-Off, pRetro-On, PLXSN, or pLNCX (Clonetech Labs, Palo Alto, Calif.), pcDNA3.1 (+/−), pcDNA/Zeo (+/−) or pcDNA3.1/Hygro (+/−) (Invitrogen), PSVL and PMSG (Pharmacia, Uppsala, Sweden), pRSVcat (ATCC 37152), pSV2dhfr (ATCC 37146) and pBC12MI (ATCC 67109). Mammalian host cells that could be used include human Hela 293, H9 and Jurkat cells, mouse NIH3T3 and C127 cells, Cos 1, Cos 7 and CV 1, quail QC1-3 cells, mouse L cells and Chinese hamster ovary (CHO) cells.

Alternatively, the gene can be expressed in stable cell lines that contain the gene integrated into a chromosome. The co-transfection with a selectable marker such as dhfr, gpt, neomycin, or hygromycin allows the identification and isolation of the transfected cells.

The transfected gene can also be amplified to express large amounts of the encoded Ig derived protein or specified portion or variant. The DHFR (dihydrofolate reductase) marker is useful to develop cell lines that carry several hundred or even several thousand copies of the gene of interest. Another useful selection marker is the enzyme glutamine synthase (GS) (Murphy, et al., Biochem. J. 227:277-279 (1991); Bebbington, et al., Bio/Technology 10: 169-175 (1992)). Using these markers, the mammalian cells are grown in selective medium and the cells with the highest resistance are selected. These cell lines contain the amplified gene(s) integrated into a chromosome. Chinese hamster ovary (CHO) and NSO cells are often used for the production of Ig derived protein or specified portion or variants.

The expression vectors pC1 and pC4 contain the strong promoter (LTR) of the Rous Sarcoma Virus (Cullen, et al., Molec. Cell. Biol. 5:438-447 (1985)) plus a fragment of the CMV-enhancer (Boshart, et al., Cell 41:521-530 (1985)). Multiple cloning sites, e.g., with the restriction enzyme cleavage sites BamHI, XbaI and Asp718, facilitate the cloning of the gene of interest. The vectors contain in addition the 3′ intron, the polyadenylation and termination signal of the rat preproinsulin gene.

Cloning and Expression in CHO Cells

The vector pC4 is used for the expression of COPD-related Ig derived protein or specified portion or variant. Plasmid pC4 is a derivative of the plasmid pSV2-dhfr (ATCC Accession No. 37146). The plasmid contains the mouse DHFR gene under control of the SV40 early promoter. Chinese hamster ovary- or other cells lacking dihydrofolate activity that are transfected with these plasmids can be selected by growing the cells in a selective medium (e.g., alpha minus MEM, Life Technologies, Gaithersburg, Md.) supplemented with the chemotherapeutic agent methotrexate. The amplification of the DHFR genes in cells resistant to methotrexate (MTX) has been well documented (see, e.g., F. W. Alt, et al., J. Biol. Chem. 253:1357-1370 (1978); J. L. Hamlin and C. Ma, Biochem. et Biophys. Acta 1097:107-143 (1990); and M. J. Page and M. A. Sydenham, Biotechnology 9:64-68 (1991)). Cells grown in increasing concentrations of MTX develop resistance to the drug by overproducing the target enzyme, DHFR, as a result of amplification of the DHFR gene. If a second gene is linked to the DHFR gene, it is usually co-amplified and over-expressed. It is known in the art that this approach can be used to develop cell lines carrying more than 1,000 copies of the amplified gene(s). Subsequently, when the methotrexate is withdrawn, cell lines are obtained that contain the amplified gene integrated into one or more chromosome(s) of the host cell.

Plasmid pC4 contains for expressing the gene of interest the strong promoter of the long terminal repeat (LTR) of the Rous Sarcoma Virus (Cullen, et al., Molec. Cell. Biol. 5:438-447 (1985)) plus a fragment isolated from the enhancer of the immediate early gene of human cytomegalovirus (CMV) (Boshart, et al., Cell 41:521-530 (1985)). Downstream of the promoter are BamHI, XbaI, and Asp718 restriction enzyme cleavage sites that allow integration of the genes. Behind these cloning sites the plasmid contains the 3′ intron and polyadenylation site of the rat preproinsulin gene. Other high efficiency promoters can also be used for the expression, e.g., the human b-actin promoter, the SV40 early or late promoters or the long terminal repeats from other retroviruses, e.g., HIV and HTLVI. Clontech's Tet-Off and Tet-On gene expression systems and similar systems can be used to express the COPD-related in a regulated way in mammalian cells (M. Gossen, and H. Bujard, Proc. Natl. Acad. Sci. USA 89: 5547-5551 (1992)). For the polyadenylation of the mRNA other signals, e.g., from the human growth hormone or globin genes can be used as well. Stable cell lines carrying a gene of interest integrated into the chromosomes can also be selected upon co-transfection with a selectable marker such as gpt, G418 or hygromycin. It is advantageous to use more than one selectable marker in the beginning, e.g., G418 plus methotrexate.

The plasmid pC4 is digested with restriction enzymes and then dephosphorylated using calf intestinal phosphatase by procedures known in the art. The vector is then isolated from a 1% agarose gel.

The DNA sequence encoding the complete COPD-related Ig derived protein or specified portion or variant is used, corresponding to HC and LC variable regions of a COPD-related Ig derived protein of the present invention, according to known method steps. Isolated nucleic acid encoding a suitable human constant region (i.e., HC and LC regions) is also used in this construct (e.g., as provided in vector p1351).

The isolated variable and constant region encoding DNA and the dephosphorylated vector are then ligated with T4 DNA ligase. E. coli HB 101 or XL-1 Blue cells are then transformed and bacteria are identified that contain the fragment inserted into plasmid pC4 using, for instance, restriction enzyme analysis.

Chinese hamster ovary (CHO) cells lacking an active DHFR gene are used for transfection. 5 μg of the expression plasmid pC4 is cotransfected with 0.5 μg of the plasmid pSV2-neo using lipofectin. The plasmid pSV2neo contains a dominant selectable marker, the neo gene from TnS encoding an enzyme that confers resistance to a group of antibiotics including G418. The cells are seeded in alpha minus MEM supplemented with 1 μg/ml G418. After 2 days, the cells are trypsinized and seeded in hybridoma cloning plates (Greiner, Germany) in alpha minus MEM supplemented with 10, 25, or 50 ng/ml of methotrexate plus 1 μg/ml G418. After about 10-14 days single clones are trypsinized and then seeded in 6-well petri dishes or 10 ml flasks using different concentrations of methotrexate (50 nM, 100 nM, 200 nM, 400 nM, 800 nM). Clones growing at the highest concentrations of methotrexate are then transferred to new 6-well plates containing even higher concentrations of methotrexate (1 mM, 2 mM, 5 mM, 10 mM, 20 mM). The same procedure is repeated until clones are obtained that grow at a concentration of 100-200 mM. Expression of the desired gene product is analyzed, for instance, by SDS-PAGE and Western blot or by reverse phase HPLC analysis.

### EXAMPLE 2

### Generation of High Affinity Human IgG Monoclonal Ig Derived Proteins Reactive With Human COPD-Related Using Transgenic Mice

Summary

Transgenic mice have been used that contain human heavy and light chain immunoglobulin genes to generate high affinity, completely human, monoclonal Ig derived proteins that can be used therapeutically to inhibit the action of COPD-related for the treatment of one or more COPD-related-mediated disease. (CBA/J x C57/BL6/J) F₂ hybrid mice containing human variable and constant region Ig derived protein transgenes for both heavy and light chains are immunized with human recombinant COPD-related (Taylor et al., Intl. Immunol. 6:579-591 (1993); Lonberg, et al., Nature 368:856-859 (1994); Neuberger, M., Nature Biotech. 14:826 (1996); Fishwild, et al., Nature Biotechnology 14:845-851 (1996)). Several fusions yielded one or more panels of completely human COPD-related reactive IgG monoclonal Ig derived proteins. The completely human anti-COPD-related Ig derived proteins are further characterized. All are IgG1κ. Such Ig derived proteins are found to have affinity constants somewhere between 1×10⁹ and 9×10¹². The unexpectedly high affinities of these fully human monoclonal Ig derived proteins make them suitable candidates for therapeutic applications in COPD-related related diseases, pathologies or disorders.

Abbreviations

BSA—bovine serum albumin

CO₂—carbon dioxide

DMSO—dimethyl sulfoxide

EIA—enzyme immunoassay

FBS—fetal bovine serum

H₂O₂—hydrogen peroxide

HRP—horseradish peroxidase

ID—interadermal

Ig—immunoglobulin

COPD-related—Chronic Obstructive Pulmonary Disease Related

IP—intraperitoneal

IV—intravenous

Mab—monoclonal Ig derived protein

OD—optical density

OPD—o-Phenylenediamine dihydrochloride

PEG—polyethylene glycol

PSA—penicillin, streptomycin, amphotericin

RT—room temperature

SQ—subcutaneous

v/v—volume per volume

w/v—weight per volume

Materials and Methods

Animals

Transgenic mice that can express human Ig derived proteins are known in the art (and are commecially available (e.g., from GenPharm International, San Jose, Calif.; Abgenix, Freemont, Calif., and others) that express human immunoglobulins but not mouse IgM or Igκ. For example, such transgenic mice contain human sequence transgenes that undergo V(D)J joining, heavy-chain class switching, and somatic mutation to generate a repertoire of human sequence immunoglobulins (Lonberg, et al., Nature 368:856-859 (1994)). The light chain transgene can be derived, e.g., in part from a yeast artificial chromosome clone that includes nearly half of the germline human Vκ region. In addition, the heavy-chain transgene can encode both human μ and human γ1(Fishwild, et al., Nature Biotechnology 14:845-851 (1996)) and/or γ3 constant regions. Mice derived from appropriate genotopic lineages can be used in the immunization and fusion processes to generate fully human monoclonal Ig derived proteins to COPD-related.

Immunization

One or more immunization schedules can be used to generate the anti-COPD-related human hybridomas. The first several fusions can be performed after the following exemplary immunization protocol, but other similar known protocols can be used. Several 14-20 week old female and/or surgically castrated transgenic male mice are immunized IP and/or ID with 1-1000 μg of recombinant human COPD-related protein emulsified with an equal volume of TITERMAX or complete Freund's adjuvant in a final volume of 100-400 μL (e.g., 200). Each mouse can also optionally receive 1-10 μg in 100 μL physiological saline at each of 2 SQ sites. The mice can then be immunized 1-7,5-12, 10-18, 17-25 and/or 21-34 days later IP (1-400 μg) and SQ (1-400 μg×2) with COPD-related protein emulsified with an equal volume of TITERMAX or incomplete Freund's adjuvant. Mice can be bled 12-25 and 25-40 days later by retro-orbital puncture without anti-coagulant. The blood is then allowed to clot at RT for one hour and the serum is collected and titered using an COPD-related protein EIA assay according to known methods. Fusions are performed when repeated injections do not cause titers to increase. At that time, the mice can be given a final IV booster injection of 1-400 μg COPD related protein diluted in 100 μL physiological saline. Three days later, the mice can be euthanized by cervical dislocation and the spleens removed aseptically and immersed in 10 mL of cold phosphate buffered saline (PBS) containing 100 U/mL penicillin, 100 μg/mL streptomycin, and 0.25 μg/mL amphotericin B (PSA). The splenocytes are harvested by sterilely perfusing the spleen with PSA-PBS. The cells are washed once in cold PSA-PBS, counted using Trypan blue dye exclusion and resuspended in RPMI 1640 media containing 25 mM Hepes.

Cell Fusion

Fusion can be carried out at a 1:1 to 1:10 ratio of murine myeloma cells to viable spleen cells according to known methods, e.g., as known in the art. As a non-limiting example, spleen cells and myeloma cells can be pelleted together. The pellet can then be slowly resuspended, over 30 seconds, in 1 mL of 50% (w/v) PEG/PBS solution (PEG molecular weight 1,450, Sigma) at 37 C. The fusion can then be stopped by slowly adding 10.5 mL of RPMI 1640 medium containing 25 mM Hepes (37 C) over 1 minute. The fused cells are centrifuged for 5 minutes at 500-1500 rpm. The cells are then resuspended in HAT medium (RPMI 1640 medium containing 25 mM Hepes, 10% Fetal Clone I serum (Hyclone), 1 mM sodium pyruvate, 4 mM L-glutamine, 10 μg/mL gentamicin, 2.5% Origen culturing supplement (Fisher), 10% 653-conditioned RPMI 1640/Hepes media, 50 μM 2-mercaptoethanol, 100 μM hypoxanthine, 0.4 μM aminopterin, and 16 μM thymidine) and then plated at 200 μL/well in fifteen 96-well flat bottom tissue culture plates. The plates are then placed in a humidified 37 C incubator containing 5% CO₂ and 95% air for 7-10 days.

Detection of Human IgG Anti-COPD-Related Ig Derived Proteins in Mouse Serum

Solid phase EIA's can be used to screen mouse sera for human IgG Ig derived proteins specific for human COPD related protein. Briefly, plates can be coated with COPD related protein at 2 μg/mL in PBS overnight. After washing in 0. 15M saline containing 0.02% (v/v) Tween 20, the wells can be blocked with 1% (w/v) BSA in PBS, 200 μL/well for 1 hour at RT. Plates are used immediately or frozen at −20 C for future use. Mouse serum dilutions are incubated on the COPD related protein coated plates at 50 μL/well at RT for 1 hour. The plates are washed and then probed with 50 μL/well HRP-labeled goat anti-human IgG, Fc specific diluted 1:30,000 in 1% BSA-PBS for 1 hour at RT. The plates can again be washed and 100 μL/well of the citrate-phosphate substrate solution (0.1M citric acid and 0.2M sodium phosphate, 0.01% H₂O₂ and 1 mg/mL OPD) is added for 15 minutes at RT. Stop solution (4N sulfuric acid) is then added at 25 pL/well and the OD's are read at 490 nm via an automated plate spectrophotometer.

Detection of Completely Human Immunoglobulins in Hybridoma Supernates

Growth positive hybridomas secreting fully human immunoglobulins can be detected using a suitable EIA. Briefly, 96 well pop-out plates (VWR, 610744) can be coated with 10 μg/mL goat anti-human IgG Fc in sodium carbonate buffer overnight at 4 C. The plates are washed and blocked with 1% BSA-PBS for one hour at 37° C. and used immediately or frozen at −20 C. Undiluted hybridoma supernatants are incubated on the plates for one hour at 37° C. The plates are washed and probed with HRP labeled goat anti-human kappa diluted 1:10,000 in 1% BSA-PBS for one hour at 37° C. The plates are then incubated with substrate solution as described above.

Determination of Fully Human Anti-COPD Related Protein Reactivity

Hybridomas, as above, can be simultaneously assayed for reactivity to COPD related protein using a suitable RIA or other assay. For example, supernatants are incubated on goat anti-human IgG Fc plates as above, washed and then probed with radiolabled COPD related protein with appropriate counts per well for 1 hour at RT. The wells are washed twice with PBS and bound radiolabled COPD related protein is quantitated using a suitable counter.

Human IgG1κ anti-COPD related protein secreting hybridomas can be expanded in cell culture and serially subcloned by limiting dilution. The resulting clonal populations can be expanded and cryopreserved in freezing medium (95% FBS, 5% DMSO) and stored in liquid nitrogen.

Isotyping

Isotype determination of the Ig derived proteins can be accomplished using an EIA in a format similar to that used to screen the mouse immune sera for specific titers. COPD related protein can be coated on 96-well plates as described above and purified Ig derived protein at 2 μg/mL can be incubated on the plate for one hour at RT. The plate is washed and probed with HRP labeled goat anti-human IgG₁ or HRP labeled goat anti-human IgG₃ diluted at 1:4000 in 1% BSA-PBS for one hour at RT. The plate is again washed and incubated with substrate solution as described above.

Binding Kinetics of Human Anti-Human COPD-Related Ig Derived Proteins With Human COPD Related Protein

Binding characteristics for Ig derived proteins can be suitably assessed using an COPD related protein capture EIA and BIAcore technology, for example. Graded concentrations of purified human COPD-related Ig derived proteins can be assessed for binding to EIA plates coated with 2 μg/mL of COPD related protein in assays as described above. The OD's can be then presented as semi-log plots showing relative binding efficiencies.

Quantitative binding constants can be obtained, e.g., as follows, or by any other known suitable method. A BIAcore CM-5 (carboxymethyl) chip is placed in a BIAcore 2000 unit. HBS buffer (0.01 M HEPES, 0.15 M NaCl, 3 mM EDTA, 0.005% v/v P20 surfactant, pH 7.4) is flowed over a flow cell of the chip at 5 μL/minute until a stable baseline is obtained. A solution (100 μL) of 15 mg of EDC (N-ethyl-N′-(3-dimethyl-aminopropyl)-carbodiimide hydrochloride) in 200 μL water is added to 100 μL of a solution of 2.3 mg of NHS (N-hydroxysuccinimide) in 200 μL water. Forty (40) μL of the resulting solution is injected onto the chip. Six μL of a solution of human COPD-related (15 μg/mL in 10 mM sodium acetate, pH 4.8) is injected onto the chip, resulting in an increase of ca. 500 RU. The buffer is changed to TBS/Ca/Mg/BSA running buffer (20 mM Tris, 0.15 M sodium chloride, 2 mM calcium chloride, 2 mM magnesium acetate, 0.5% Triton X-100, 25 μg/mL BSA, pH 7.4) and flowed over the chip overnight to equilibrate it and to hydrolyze or cap any unreacted succinimide esters.

Ig derived proteins are dissolved in the running buffer at 33.33, 16.67, 8.33, and 4.17 nM. The flow rate is adjusted to 30 μL/min and the instrument temperature to 25 C. Two flow cells are used for the kinetic runs, one on which COPD related protein had been immobilized (sample) and a second, underivatized flow cell (blank). 120 μL of each Ig derived protein concentration is injected over the flow cells at 30 μL/min (association phase) followed by an uninterrupted 360 seconds of buffer flow (dissociation phase). The surface of the chip is regenerated (Chronic Obstructive Pulmonary Disease Related/Ig derived protein complex dissociated) by two sequential injections of 30 μL each of 2 M guanidine thiocyanate.

Analysis of the data is done using BIA evaluation 3.0 or CLAMP 2.0, as known in the art. For each Ig derived protein concentration the blank sensogram is subtracted from the sample sensogram. A global fit is done for both dissociation (k_{d}, sec⁻¹) and association (kₐ, mol⁻¹ sec⁻¹) and the dissociation constant (K_{D}, mol) calculated (k_{d}/kₐ). Where the Ig derived protein affinity is high enough that the RUs of Ig derived protein captured are >100, additional dilutions of the Ig derived protein are run.

Results and Discussion

Generation of Anti-Human COPD-related Monoclonal Ig derived Proteins

Several fusions are performed and each fusion is seeded in 15 plates (1440 wells/fusion) that yield several dozen Ig derived proteins specific for human COPD related protein. Of these, some are found to consist of a combination of human and mouse Ig chains. The remaining hybridomas secret anti-COPD-related Ig derived proteins consisting solely of human heavy and light chains. Of the human hybridomas all are expected to be IgG1κ.

Binding Kinetics of Human Anti-Human COPD-Related Ig Derived Proteins

ELISA analysis confirms that purified Ig derived protein from most or all of these hybridomas bind COPD related protein in a concentration-dependent manner. FIGS. 1-2 show the results of the relative binding efficiency of these Ig derived proteins. In this case, the avidity of the Ig derived protein for its cognate antigen (epitope) is measured. It should be noted that binding COPD related protein directly to the EIA plate can cause denaturation of the protein and the apparent binding affinities cannot be reflective of binding to undenatured protein. Fifty percent binding is found over a range of concentrations.

Quantitative binding constants are obtained using BIAcore analysis of the human Ig derived proteins and reveals that several of the human monoclonal Ig derived proteins are very high affinity with K_{D} in the range of 1×10⁻⁹ to 7×10⁻¹².

Conclusions

Several fusions are performed utilizing splenocytes from hybrid mice containing human variable and constant region Ig derived protein transgenes that are immunized with human COPD related protein. A set of several completely human COPD related protein reactive IgG monoclonal Ig derived proteins of the IgG1κ isotype are generated. The completely human anti-COPD related protein Ig derived proteins are further characterized. Several of generated Ig derived proteins have affinity constants between 1×10⁹ and 9×10¹². The unexpectedly high affinities of these fully human monoclonal Ig derived proteins make them suitable for therapeutic applications in COPD related protein-dependent diseases, pathologies or related conditions.

It will be clear that the invention can be practiced otherwise than as particularly described in the foregoing description and examples.

Numerous modifications and variations of the present invention are possible in light of the above teachings and, therefore, are within the scope of the appended claims.

### REFERENCES

1. Glennie M J, Johnson P W: Clinical trials of antibody therapy. Immunol Today 2000, 21:403-410.

2. Barnes P J: Mechanisms in COPD: differences from asthma. Chest 2000, 117:10S-14S.

3. Leckie M J, ten Brinke A, Khan J, Diamant Z, O'Connor B J, Walls C M, Mathur A K, Cowley H C, Chung K F, Djukanovic R, et al.: Effects of an interleukin-5 blocking monoclonal antibody on eosinophils, airway hyper-responsiveness, and the late asthmatic response. Lancet 2000, 356:2144-2148.

4. Bryan S A, O'Connor B J, Matti S, Leckie M J, Kanabar V, Khan J, Warrington S J, Renzetti L, Rames A, Bock J A, et al.: Effects of recombinant human interleukin-12 on eosinophils, airway hyper-responsiveness, and the late asthmatic response. Lancet 2000, 356:2149-2153.

5. Breedveld F C: Therapeutic monoclonal antibodies. Lancet 2000, 355:735-740.

6. Hoogenboom H R, Chames P: Natural and designer binding sites made by phage display technology. Immunol Today 2000, 21:371-378.

7. Green L L: Antibody engineering via genetic engineering of the mouse: XenoMouse strains are a vehicle for the facile generation of therapeutic human monoclonal antibodies. J Immunol Methods 1999, 231:11-23.

8. Reilly R M, Domingo R, Sandhu J: Oral delivery of antibodies. Future pharmacokinetic trends. Clin Pharmacokinet 1997, 32:313-323.

9. Russell D A: Feasibility of antibody production in plants for human therapeutic use. Curr Top Microbiol Immunol 1999, 240:119-138.

10. Little M, Kipriyanov S M, Le Gall F, Moldenhauer G: Of mice and men: hybridoma and recombinant antibodies. Immunol Today 2000, 21:364-370.

11. Clark M: Antibody humanization: a case of the ‘Emperor's new clothes’? Immunol Today 2000, 21:397-402.

12. Kipriyanov S M, Little M: Generation of recombinant antibodies. Mol Biotechnol 1999,12:173-201.

13. Laurincova B: Interleukin-1 family: from genes to human disease. Acta Univ Palacki Olomuc Fac Med 2000, 143:19-29.

14. Idriss H T, Naismith J H: TNF alpha and the TNF receptor superfamily: structure-function relationship(s). Microsc Res Tech 2000, 50:184-195.

15. Maini R N, Taylor P C: Anti-cytokine therapy for rheumatoid arthritis. Annu Rev Med 2000, 51:207-229.

16. Raza A: Anti-TNF therapies in rheumatoid arthritis, Crohn's disease, sepsis, and myelodysplastic syndromes. Microsc Res Tech 2000, 50:229-235.

17. Mikula C A: Anti-TNF alpha: new therapy for Crohn's disease. Gastroenterol Nurs 1999, 22:245-248.

18. Kuhn C, Homer R J, Zhu Z, Ward N, Flavell R A, Geba G P, Elias J A: Airway hyperresponsiveness and airway obstruction in transgenic mice. Morphologic correlates in mice overexpressing interleukin (IL)-11 and IL-6 in the lung. Am J Respir Cell Mol Biol 2000, 22:289-295.

19. Murphy P M: Neutrophil receptors for interleukin-8 and related CXC chemokines. Semin Hematol 1997,34:311-318.

20. Lohner M, Schwab G, Krueger G: Clinical trial results: a fully human anti-IL-8 antibody in patients with moderate to severe psoriasis. J. Am. Acad. Dermatol. 2001, 44:P516.

21. Jeffery P K: Lymphocytes, chronic bronchitis and chronic obstructive pulmonary disease. Novartis Found Symp 2001, 234:149-161; discussion 161-148.

22. Tohda Y, Kubo H, Nakajima S, Fukuoka M: Effect of anti-ICAM-1 on bronchial response: bronchoalveolar lavage fluid (BALF) and ultrastructural changes of bronchial epithelium in guinea pigs with dual phase bronchial response. Methods Find Exp Clin Pharmacol 1999, 21:541-547.

23. Lin K C, Castro A C: Very late antigen 4 (VLA4) antagonists as anti-inflammatory agents. Curr Opin Chem Biol 1998, 2:453-457.

24. Cataldo D, Munaut C, Noel A, Frankenne F, Bartsch P, Foidart J M, Louis R: MMP-2- and MMP-9-linked gelatinolytic activity in the sputum from patients with asthma and chronic obstructive pulmonary disease. Int Arch Allergy Immunol 2000, 123:259-267.

25. Zheng T, Zhu Z, Wang Z, Homer R J, Ma B, Riese R J, Chapman H A, Shapiro S D, Elias J A: Inducible targeting of IL-13 to the adult lung causes matrix metalloproteinase- and cathepsin-dependent emphysema. J Clin Invest 2000, 106:1081-1093.

26. Kon O M, Kay A B: T cells and chronic asthma. Int Arch Allergy Immunol 1999, 118:133-135.

27. Kon O M, Sihra B S, Compton C H, Leonard T B, Kay A B, Barnes N C: Randomised, dose-ranging, placebo-controlled study of chimeric antibody to CD4 (keliximab) in chronic severe asthma. Lancet 1998, 352:1109-1113.

28. Li L, Crowley M, Nguyen A, Lo D: Ability of a nondepleting anti-CD4 antibody to inhibit Th2 responses and allergic lung inflammation is independent of coreceptor function. J Immunol 1999, 163:6557-6566.

29. Djukanovic R: The role of co-stimulation in airway inflammation. Clin Exp Allergy 2000, 30:46-50.

30. Mathur M, Herrmann K, Qin Y, Gulmen F, Li X, Krimins R, Weinstock J, Elliott D, Bluestone J A, Padrid P: CD28 interactions with either CD80 or CD86 are sufficient to induce allergic airway inflammation in mice. Am J Respir Cell Mol Biol 1999, 21:498-509.

31. Padrid P A, Mathur M, Li X, Hermann K, Qin Y, Cattamanchi A, Weinstock J, Elliott D, Sperling A I, Bluestone J A: CTLA4Ig inhibits airway eosinophilia and hyperresponsiveness by regulating the development of Th1/Th2 subsets in a murine model of asthma. Am J Respir Cell Mol Biol 1998, 18:453-462.

32. Jaffar Z H, Stanciu L, Pandit A, Lordan J, Holgate S T, Roberts K: Essential role for both CD80 and CD86 costimulation, but not CD40 interactions, in allergen-induced Th2 cytokine production from asthmatic bronchial tissue: role for alphabeta, but not gammadelta, T cells. J Immunol 1999, 163:6283-6291.

33. Jaffar Z, Roberts K, Pandit A, Linsley P, Djukanovic R, Holgate S: B7 costimulation is required for IL-5 and IL-13 secretion by bronchial biopsy tissue of atopic asthmatic subjects in response to allergen stimulation. Am J Respir Cell Mol Biol 1999, 20:153-162.

34. Wong W S, Koh DS: Advances in immunopharmacology of asthma. Biochem Pharmacol 2000, 59:1323-1335.

35. Borish L C, Nelson H S, Lanz M J, Claussen L, Whitmore J B, Agosti J M, Garrison L: Interleukin-4 receptor in moderate atopic asthma. A phase I/II randomized, placebo-controlled trial. Am J Respir Crit Care Med 1999, 160:1816-1823.

36. Enbrel, Nuvance failures shake Immunex stock, cause uncertainty. NewsEdge Insight 2001.

37. Kips J: Results of a Phase I trial with SCH55700, a humanized anti-IL-5 antibody, in severe persistent asthma. AM. J Respir. Crit. Care Med. 2000, 161:A505.

38. Wills-Karp M: IL-12/1L-13 axis in allergic asthma. J Allergy Clin Immunol 2001, 107:9-18.

39. Zhou Y, McLane M, Levitt RC: Interleukin-9 as a therapeutic target for asthma. Respir. Res. 2001, 2:in press.

40. Wills-Karp M, Luyimbazi J, Xu X, Schofield B, Neben T Y, Karp C L, Donaldson D D: Interleukin-13: central mediator of allergic asthma (see comments). Science 1998, 282:2258-2261.

41. Grunig G, Warnock M, Wakil A E, Venkayya R, Brombacher F, Rennick D M, Sheppard D, Mohrs M, Donaldson D D, Locksley R M, et al.: Requirement for IL-13 independently of IL-4 in experimental asthma (see comments). Science 1998, 282:2261-2263.

42. Kung T T, Crawley Y, Lou B, Minnicozzi M, Jones H, Bgan R W, Kreutner W, R. W. C: Effect of anti-mIL-9 antibody on the development of pulmonary eosinophilia and airway hyperresponsiveness in allergic mice (abstract). Am. J. Respir. Crit. Care Med. 2000, 161:A844.

43. Mclane M P, Tepper J, Weiss C, Timer Y, Tibin P, Tu aD, Zhou Y, Jaczku A, Nicolaides N C, Levitt R C: Lung delivery of blocking IL9 antibody inhibits airway hyperresponsiveness, Bal eosinophilia, mucin production, serum IgE elevation to natural antigens in a murine model of asthma (abstract). Am. J. Respir. Crit. Care Med. 2000, 161:A593.

44. Barnes P J: Anti-IgE therapy in asthma: rationale and therapeutic potential. Int Arch Allergy Immunol 2000, 123:196-204.

45. Fahy J V: Reducing IgE levels as a strategy for the treatment of asthma. Clin Exp Allergy 2000, 30:16-21.

46. Milgrom H, Fick R B, Su J Q, Reimann J D, Bush R K, Watrous M L, Metzger W J: Treatment of allergic asthma with monoclonal anti-IgE antibody. rhuMAb-E25 Study Group. N Engl J Med 1999, 341:1966-1973.

47. Dasic G. Juillard P, Graber P, Herren S, Angell T, Knowles R, Bonnefoy J Y, Kosco-Vilbois M H, Chvatchko Y: Critical role of CD23 in allergen-induced bronchoconstriction in a murine model of allergic asthma. Eur J Immunol 1999, 29:2957-2967.

48. Mayer R J, Bolognese B J, Al-Mahdi N, Cook R M, Flamberg P L, Hansbury M J, Khandekar S, Appelbaum E, Faller A, Marshall L A: Inhibition of CD23 processing correlates with inhibition of IL-4-stimulated IgE production in human PBL and hu-PBL-reconstituted SCID mice. Clin Exp Allergy 2000, 30:719-727.

49. Haczku A, Takeda K, Hamelmann E, Loader J, Joetham A, Redai I, Irvin CG, Lee J J, Kikutani H, Conrad D, et al.: CD23 exhibits negative regulatory effects on allergic sensitization and airway hyperresponsiveness. Am J Respir Crit Care Med 2000, 161:952-960.

50. Gangur V, Oppenheim J J: Are chemokines essential or secondary participants in allergic responses? Ann Allergy Asthma Immunol 2000, 84:569-579; quiz 579-581.

51. Rothenberg ME, Zimmermann N, Mishra A, Brandt E, Birkenberger L A, Hogan S P, Foster P S: Chemokines and chemokine receptors: their role in allergic airway disease. J Clin Immunol 1999, 19:250-265.

52. Ying S, Meng Q, Zeibecoglou K, Robinson D S, Macfarlane A, Humbert M, Kay A B: Eosinophil chemotactic chemokines (eotaxin, eotaxin-2, RANTES, monocyte chemoattractant protein-3 (MCP-3), and MCP-4), and C-C chemokine receptor 3 expression in bronchial biopsies from atopic and nonatopic (Intrinsic) asthmatics. J Immunol 1999, 163:6321-6329.

53. Gordon J R: Monocyte chemoattractant peptide-1 expression during cutaneous allergic reactions in mice is mast cell dependent and largely mediates the monocyte recruitment response. J Allergy Clin Immunol 2000, 106:110-116.

54. Stafford S, Li H, Forsythe P A, Ryan M, Bravo R, Alam R: Monocyte chemotactic protein-3 (MCP-3)/fibroblast-induced cytokine (FIC) in eosinophilic inflammation of the airways and the inhibitory effects of an anti-MCP-3/FIC antibody. J Immunol 1997, 158:4953-4960.

55. Rothenberg M E, MacLean J A, Pearlman E, Luster A D, Leder P: Targeted disruption of the chemokine eotaxin partially reduces antigen-induced tissue eosinophilia. J Exp Med 1997, 185:785-790.

56. Schleimer R P, Bochner B S: The role of adhesion molecules in allergic inflammation and their suitability as targets of antiallergic therapy. Clin Exp Allergy 1998, 28:15-23.

57. Wolyniec W W, De Sanctis G T, Nabozny G, Torcellini C, Haynes N, Joetham A, Gelfand E W, Drazen J M, Noonan T C: Reduction of antigen-induced airway hyperreactivity and eosinophilia in ICAM-1-deficient mice. Am J Respir Cell Mol Biol 1998, 18:777-785.

58. Ricci M, Matucci A, Rossi O: New advances in the pathogenesis and therapy of bronchial asthma. Ann Ital Med Int 1998, 13:93-110.

59. Rosenwasser L J: Biologic activities of IL-1 and its role in human disease. J Allergy Clin Immunol 1998, 102:344-350.

60. Sim T C, Hilsmeier K A, Reece L M, Grant J A, Alam R: Interleukin-1 receptor antagonist protein inhibits the synthesis of IgE and proinflammatory cytokines by allergen-stimulated mononuclear cells. Am J Respir Cell Mol Biol 1994, 11:473-479.

61. Okada S, Inoue H, Yamauchi K, Iijima H, Ohkawara Y, Takishima T, Shirato K: Potential role of interleukin-1 in allergen-induced late asthmatic reactions in guinea pigs: suppressive effect of interleukin-1 receptor antagonist on late asthmatic reaction. J Allergy Clin Immunol 1995, 95:1236-1245.

62. Barnes P J: Chronic obstructive pulmonary disease: new opportunities for drug development. Trends Pharmacol Sci 1998, 19:415-423.

63. Metcalf J P, Thompson A B, Gossman G L, Nelson K J, Koyama S, Rennard S I, Robbins R A: Gc-globulin functions as a cochemotaxin in the lower respiratory tract. A potential mechanism for lung neutrophil recruitment in cigarette smokers. Am Rev Respir Dis 1991, 143:844-849.

64. Noguera A, Busquets X, Sauleda J, Villaverde J M, MacNee W, Agusti A G: Expression of adhesion molecules and G proteins in circulating neutrophils in chronic obstructive pulmonary disease. Am J Respir Crit Care Med 1998, 158:1664-1668.

65. Symon F A, Wardlaw A J: Selectins and their counter receptors: a bitter sweet attraction. Thorax 1996, 51:1155-1157.

66. Gonzalez S, Hards J, van Eeden S, Hogg J C: The expression of adhesion molecules in cigarette smoke-induced airways obstruction. Eur Respir J 1996, 9:1995-2001.

67. Nadel J A: Role of neutrophil elastase in hypersecretion during COPD exacerbations, and proposed therapies. Chest 2000, 117:386S-389S.

68. Wills-Karp M: Immunologic basis of antigen-induced airway hyperresponsiveness. Annu Rev Immunol 1999, 17:255-281.

69. Conroy D M, Humbles A A, Rankin S M, Palframan R T, Collins P D, Griffiths-Johnson D A, Jose P J, Williams T J: The role of the eosinophil-selective chemokine, eotaxin, in allergic and non-allergic airways inflammation. Mem Inst Oswaldo Cruz 1997, 92 Suppl 2:183-191.

70. Bertrand C P, Ponath P D: CCR3 blockade as a new therapy for asthma. Expert Opin Investig Drugs 2000, 9:43-52.

What is claimed is:

1. An isolated chronic obstructive pulmonary disease (COPD) related Ig derived protein or specified portion or variant, comprising at least one immuoglobulin complementarity determining region (CDR) or at least one ligand binding region (LBR) that specifically binds at least one COPD related protein, wherein
(a) said COPD-related Ig derived protein or specified portion or variant specifically binds at least one epitope comprising at least 1-3, to the entire amino acid sequence, selected from the group consisting of a human tissue necrosis factor alpha (TNF), an interleukin-6 (IL-6), an interleukin-8 (IL-8); an epidermal growth factor (EGF); a CD-8 or a CD-18; or
(b) said COPD-related Ig derived protein or specified portion or variant comprises at least COPD-related protein binding region selected from at least 1-3 amino acids selected from the group consisting of a human tissue necrosis factor alpha (TNF) ligand or receptor, an interleukin-6 (IL-6) receptor or ligand, an interleukin-8 (IL-8) receptor or ligand; an epidermal growth factor (EGF) receptor or ligand; a CD-8 receptor or ligand; or a CD-18 receptor or ligand.

2. An COPD-related human Ig derived protein or specified portion or variant according to claim 1, wherein
(a) said COPD-related Ig derived protein or specified portion or variant specifically binds at least one epitope comprising at least 1-3, to the entire amino acid sequence, selected from the group consisting of: from 1-80 to 80-157 of SEQ ID NO:1; from 77-116 to 117-233 of SEQ ID NO:2; from 28-106 to 107-212 of SEQ ID NO:3; from 21-50 to 51-99 of SEQ ID NO:4; from 23-605 to 606-1207 of SEQ ID NO:5; from 22-118 to 119-235 of SEQ ID NO:6; from 1-23 to 24-45 of SEQ ID NO:7; from 1-19 to 20-37 of SEQ ID NO:8; from 22-105 to 106-210 of SEQ ID NO:9; from 1-123 to 124-246 of SEQ ID NO:10; from 1-40 to 40-80 of SEQ ID NO:11; from 23-385 to 386-769 of SEQ ID NO:12; or
(b) said COPD-related Ig derived protein or specified portion or variant comprises at least COPD-related protein binding region selected from at least 1-3 amino acids selected from the group consisting of 22455 of SEQ ID NO:13; 1-53 of SEQ ID NO:14; 1-350 of SEQ ID NO:15; 1-360 of SEQ ID NO:16; 25-1210 of SEQ ID NO:17.

3. An COPD-related human Ig derived protein or specified portion or variant according to claim 1, wherein said human Ig derived protein or specified portion or variant binds COPD-related with an affinity of at least 10⁻⁹ M.

4. An COPD-related human Ig derived protein or specified portion or variant according to claim 1, wherein said human Ig derived protein or specified portion or variant binds COPD-related with an affinity of at least 10⁻¹¹ M.

5. An COPD-related human Ig derived protein or specified portion or variant, according to claim 1, wherein said human Ig derived protein or specified portion or variant binds with an affinity of at least 10⁻¹² M.

6. An COPD-related human Ig derived protein or specified portion or variant according to claim 1, wherein said human Ig derived protein or specified portion or variant substantially neutralizes at least one activity of at least one COPD-related.

7. An isolated COPD-related human Ig derived protein encoding nucleic acid, comprising a nucleic acid that hybridizes under stringent conditions, or has at least 95% identity, to a nucleic acid encoding a COPD-related Ig derived protein according to claim 1.

8. An isolated COPD-related human Ig derived protein or specified portion or variant, comprising an isolated human Ig derived protein or specified portion or variant encoded by a nucleic acid according to claim 7.

9. An COPD-related human Ig derived protein encoding nucleic acid composition, comprising an isolated nucleic acid according to claim 7 and a carrier or diluent.

10. A human Ig derived protein vector, comprising a nucleic acid according to claim 7.

11. A human Ig derived protein vector according to claim 10, wherein said vector comprises at least one promoter selected from the group consisting of a late or early SV40 promoter, a CMV promoter, an HSV tk promoter, a pgk (phosphoglycerate kinase) promoter, a human immunoglobulin promoter, or an EF-1 alpha promoter.

12. A human Ig derived protein vector according to claim 10, wherein said vector comprises at least one selection gene or portion thereof selected from at least one of methotrexate (MTX), dihydrofolate reductase (DHFR), green fluorescent protein (GFP), neomycin (G418), or glutamine synthetase (GS).

13. A mammalian host cell comprising an isolated nucleic acid according to claim 7.

14. A host cell according to claim 13, wherein said host cell is at least one selected from COS-1, COS-7, HEK293, BHK21, CHO, BSC-1, Hep G2, 653, SP2/0, 293, HeLa, myeloma, or lymphoma cells, or any derivative, immortalized or transformed cell thereof.

15. A method for producing at least one COPD-related human Ig derived protein or specified portion or variant, comprising translating a nucleic acid according to claim 7 or an endogenous nucleic acid that hybridizes thereto under stringent conditions, under conditions in vitro, in vivo or in situ, such that the COPD-related human Ig derived protein or specified portion or variant is expressed in detectable or recoverable amounts.

16. An COPD-related human Ig derived protein or specified portion or variant composition, comprising at least one isolated COPD-related human Ig derived protein or specified portion or variant according to claim 1, and a carrier or diluent.

17. A composition according to claim 16, wherein said carrier or diluent is pharmaceutically acceptable.

18. A composition according to claim 16, further comprising at least one compound or protein selected from at least one of a TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

19. A method for treating a COPD-related condition in a cell, tissue, organ or animal, comprising
(a) contacting or administering a COPD modulating effective amount of at least one COPD-related human Ig derived protein or specified portion or variant according to claim 1 with, or to, said cell, tissue, organ or animal.

20. A method according to claim 19, wherein said animal is a primate.

21. A method according to claim 20, wherein said primate is a monkey or a human.

22. A method according to claim 19, wherein said COPD related condition is at least one selected from COPD, emphysema, asthma, chronic bronchitis or airflow obstruction.

23. A method according to claim 19, wherein said effective amount is 0.001-50 mg/kilogram of said cells, tissue, organ or animal.

24. A method according to claim 19, wherein said contacting or said administrating is by at least one mode selected from intravenous, intramuscular, bolus, intraperitoneal, subcutaneous, respiratory, inhalation, nasal, vaginal, rectal, buccal, sublingual, intranasal, subdermal, or transdermal.

25. A method according to claim 19, further comprising administering, prior, concurrently or after said (a) contacting or administering, at least one composition comprising a therapeutically effective amount of at least one compound or protein selected from at least one of a TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

26. A medical device, comprising at least one COPD-related human Ig derived protein or specified portion or variant according to claim 1, wherein said device is suitable to contacting or administerting said at least one COPD-related human Ig derived protein or specified portion or variant by at least one mode selected from intravenous, intramuscular, bolus, intraperitoneal, subcutaneous, respiratory, inhalation, nasal, vaginal, rectal, buccal, sublingual, intranasal, subdermal, or transdermal.

27. A human immunoglobulin light chain COPD-related or portion thereof, comprising at least one portion of a variable region comprising at least one human Ig derived protein fragment according to claim 1.

28. A human immunoglobulin heavy chain or portion thereof, comprising at least one portion of a variable region comprising at least one COPD-related human Ig derived protein fragment according to claim 1.

29. A human Ig derived protein or specified portion or variant thereof, wherein said human Ig derived protein or specified portion or variant binds the same epitope or antigenic region as a COPD-related human Ig derived protein or specified portion or variant according to claim 1.

30. A formulation comprising at least one COPD-related human Ig derived protein or specified portion or variant according to claim 1, and at least one selected from sterile water, sterile buffered water, or at least one preservative selected from the group consisting of phenol, m-cresol, p-cresol, o-cresol, chlorocresol, benzyl alcohol, alkylparaben, benzalkonium chloride, benzethonium chloride, sodium dehydroacetate and thimerosal, or mixtures thereof in an aqueous diluent.

31. A formulation of claim 30, wherein the concentration of COPD-related human Ig derived protein or specified portion or variant is about 0.1 mg/ml to about 100 mg/ml.

32. A formulation of claim 30, further comprising an isotonicity agent.

33. A formulation of claim 30, further comprising a physiologically acceptable buffer.

34. A formulation comprising at least one COPD-related human Ig derived protein or specified portion or variant according to claim 1 in lyophilized form in a first container, and an optional second container comprising at least one of sterile water, sterile buffered water, or at least one preservative selected from the group consisting of phenol, m-cresol, p-cresol, o-cresol, chlorocresol, benzyl alcohol, alkylparaben, benzalkonium chloride, benzethonium chloride, sodium dehydroacetate and thimerosal, or mixtures thereof in an aqueous diluent.

35. A formulation of claim 34, wherein the concentration of COPD-related human Ig derived protein or specified portion or variant is reconsitituted to a concentration of about 0.1 mg/ml to about 500 mg/ml.

36. A formulation of claim 34, further comprising an isotonicity agent.

37. A formulation of claim 34, further comprising a physiologically acceptable buffer.

38. A method of treating at least one COPD-related mediated condition, comprising administering to a patient in need thereof a formulation according to claim 34.

39. An article of manufacture for human pharmaceutical use, comprising packaging material and a container comprising a solution or a lyophilized form of at least one COPD-related human Ig derived protein or specified portion or variant according to claim 1.

40. The article of manufacture of claim 39, wherein said container is a glass or plastic container having a stopper for multi-use administration.

41. The article of manufacture of claim 39, wherein said container is a blister pack, capable of being punctured and used in intravenous, intramuscular, bolus, intraperitoneal, subcutaneous, respiratory, inhalation, nasal, vaginal, rectal, buccal, sublingual, intranasal, subdermal, or transdermal administration.

42. The article of manufacture of claim 39, wherein said container is a component of a intravenous, intramuscular, bolus, intraperitoneal, subcutaneous, respiratory, inhalation, nasal, vaginal, rectal, buccal, sublingual, intranasal, subdermal, or transdermal delivery device or system.

43. The article of manufacture of claim 39, wherein said container is a component of an injector or pen-injector device or system for intravenous, intramuscular, bolus, intraperitoneal, subcutaneous, respiratory, inhalation, nasal, vaginal, rectal, buccal, sublingual, intranasal, subdermal, or transdermal.

44. A method for preparing a formulation of at least one COPD-related human Ig derived protein or specified portion or variant, comprising admixing at least one COPD-related human Ig derived protein or specified portion or variant according to claim 1 in at least one buffer containing saline or a salt.

45. A method for producing at least one COPD-related human Ig derived protein or specified portion or variant according to claim 1, comprising providing a host cell, transgenic animal, transgenic plant or plant cell capable of expressing in recoverable amounts said human Ig derived protein or specified portion or variant.

46. A method according to claim 45, wherein said host cell is a mammalian cell, a plant cell or a yeast cell.

47. A method according to claim 45, wherein said transgenic animal is a mammal.

48. A method according to claim 47, wherein said transgenic mammal is selected from a goat, a cow, a sheep, a horse, and a non-human primate.

49. A transgenic animal or plant expressing at least one human Ig derived protein according to claim 1.

50. At least one COPD-related human Ig derived protein or specified portion or variant produced by a method according to claim 45.

51. A method for treating at least one COPD-related mediated disorder, comprising
(a) administering an effective amount of a composition or pharmaceutical composition comprising at least one COPD-related human Ig derived protein or specified portion or variant to a cell, tissue, organ, animal or patient in need of such modulation, treatment or therapy; and
(b) further administering, before concurrently, and/or after said administering in (a) above, at least one selected from at least one TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropoietin, a filgrastim, a sargramostim, an immunizing agent, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, adonepezil, a tacrine, an asthma medication, a beta agonist, an inhaled steroid, a leukotriene inhibitor, a methylxanthine, a cromolyn, an epinephrine or analog, a dornase alpha, or a cytokine, a cytokine antagonist.

52. Any invention described herein.

53. An isolated chronic obstructive pulmonary disease (COPD) related Ig derived protein or specified portion or variant, comprising at least one immnuoglobulin complementarity determining region (CDR) or at least one ligand binding region (LBR) that specifically binds at least one COPD related protein, wherein
(a) said COPD-related Ig derived protein or specified portion or variant specifically binds at least one epitope comprising at least 1-3, to the entire amino acid sequence, selected from the group consisting of a human CXCR1, CXCR2, MCP-1, C5a Gc globulin, ICAM-1, E-selectin, IL-1, Neutrophil elastase, a cathepsin, an MMP; or
(b) (b) said COPD-related Ig derived protein or specified portion or variant comprises at least COPD-related protein binding region selected from at least 1-3 amino acids selected from the group consisting of a human a human CXCR1, CXCR2, MCP-1, C5a Gc globulin, ICAM-1, E-selectin, IL-1, Neutrophil elastase, a cathepsin, an MMP.

54. An COPD-related human Ig derived protein or specified portion or variant according to claim 53, wherein said human Ig derived protein or specified portion or variant binds COPD-related with an affinity of at least 10⁻⁹ M.

55. An COPD-related human Ig derived protein or specified portion or variant according to claim 53, wherein said human Ig derived protein or specified portion or variant binds COPD-related with an affinity of at least 10⁻¹¹ M.

56. An COPD-related human Ig derived protein or specified portion or variant, according to claim 53, wherein said human Ig derived protein or specified portion or variant binds with an affinity of at least 10⁻¹² M.

57. An COPD-related human Ig derived protein or specified portion or variant according to claim 53, wherein said human Ig derived protein or specified portion or variant substantially neutralizes at least one activity of at least one COPD-related.

58. An isolated COPD-related human Ig derived protein encoding nucleic acid, comprising a nucleic acid that hybridizes under stringent conditions, or has at least 95% identity, to a nucleic acid encoding a COPD-related Ig derived protein according to claim 53.

59. An isolated COPD-related human Ig derived protein or specified portion or variant, comprising an isolated human Ig derived protein or specified portion or variant encoded by a nucleic acid according to claim 58.

60. An COPD-related human Ig derived protein encoding nucleic acid composition, comprising an isolated nucleic acid according to claim 58 and a carrier or diluent.

61. A human Ig derived protein vector, comprising a nucleic acid according to claim 58.

62. A human Ig derived protein vector according to claim 61, wherein said vector comprises at least one promoter selected from the group consisting of a late or early SV40 promoter, a CMV promoter, an HSV tk promoter, a pgk (phosphoglycerate kinase) promoter, a human immunoglobulin promoter, or an EF-1 alpha promoter.

63. A human Ig derived protein vector according to claim 61, wherein said vector comprises at least one selection gene or portion thereof selected from at least one of methotrexate (MTX), dihydrofolate reductase (DHFR), green fluorescent protein (GFP), neomycin (G418), or glutamine synthetase (GS).

64. A mammalian host cell comprising an isolated nucleic acid according to claim 58.

65. A host cell according to claim 64, wherein said host cell is at least one selected from COS-1, COS-7, HEK293, BHK21, CHO, BSC-1, Hep G2, 653, SP2/0, 293, HeLa, myeloma, or lymphoma cells, or any derivative, immortalized or transformed cell thereof.

66. A method for producing at least one COPD-related human Ig derived protein or specified portion or variant, comprising translating a nucleic acid according to claim 58 or an endogenous nucleic acid that hybridizes thereto under stringent conditions, under conditions in vitro, in vivo or in situ, such that the COPD-related human Ig derived protein or specified portion or variant is expressed in detectable or recoverable amounts.

67. An COPD-related human Ig derived protein or specified portion or variant composition, comprising at least one isolated COPD-related human Ig derived protein or specified portion or variant according to claim 53, and a carrier or diluent.

68. A composition according to claim 67, wherein said carrier or diluent is pharmaceutically acceptable.

69. A composition according to claim 67, further comprising at least one compound or protein selected from at least one of a TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

70. A method for treating a COPD-related condition in a cell, tissue, organ or animal, comprising
(a) contacting or administering a COPD modulating effective amount of at least one COPD-related human Ig derived protein or specified portion or variant according to claim 53 with, or to, said cell, tissue, organ or animal.

71. A method according to claim 70, wherein said animal is a primate.

72. A method according to claim 71, wherein said primate is a monkey or a human.

73. A method according to claim 70, wherein said COPD related condition is at least one selected from COPD, emphysema, asthma, chronic bronchitis or airflow obstruction.

74. A method according to claim 70, wherein said effective amount is 0.001-50 mg/kilogram of said cells, tissue, organ or animal.

75. A method according to claim 70, wherein said contacting or said administrating is by at least one mode selected from intravenous, intramuscular, bolus, intraperitoneal, subcutaneous, respiratory, inhalation, nasal, vaginal, rectal, buccal, sublingual, intranasal, subdermal, or transdermal.

76. A method according to claim 70, further comprising administering, prior, concurrently or after said (a) contacting or administering, at least one composition comprising a therapeutically effective amount of at least one compound or protein selected from at least one of a TNF antagonist, an antirheumatic, a muscle relaxant, a narcotic, a non-steroid anti-inflammatory drug (NSAID), an analgesic, an anesthetic, a sedative, a local anethetic, a neuromuscular blocker, an antimicrobial, an antipsoriatic, a corticosteriod, an anabolic steroid, a diabetes related agent, a mineral, a nutritional, a thyroid agent, a vitamin, a calcium related hormone, an antidiarrheal, an antitussive, an antiemetic, an antiulcer, a laxative, an anticoagulant, an erythropieitin, a filgrastim, a sargramostim, an immunization, an immunoglobulin, an immunosuppressive, a growth hormone, a hormone replacement drug, an estrogen receptor modulator, a mydriatic, a cycloplegic, an alkylating agent, an antimetabolite, a mitotic inhibitor, a radiopharmaceutical, an antidepressant, antimanic agent, an antipsychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog, dornase alpha, a cytokine, a cytokine antagonist.

77. A medical device, comprising at least one COPD-related human Ig derived protein or specified portion or variant according to claim 53, wherein said device is suitable to contacting or administerting said at least one COPD-related human Ig derived protein or specified portion or variant by at least one mode selected from intravenous, intramuscular, bolus, intraperitoneal, subcutaneous, respiratory, inhalation, nasal, vaginal, rectal, buccal, sublingual, intranasal, subdermal, or transdermal.

78. A human immunoglobulin light chain COPD-related or portion thereof, comprising at least one portion of a variable region comprising at least one human Ig derived protein fragment according to claim 53.

79. A human immunoglobulin heavy chain or portion thereof, comprising at least one portion of a variable region comprising at least one COPD-related human Ig derived protein fragment according to claim 53.

80. A human Ig derived protein or specified portion or variant thereof, wherein said human Ig derived protein or specified portion or variant binds the same epitope or antigenic region as a COPD-related human Ig derived protein or specified portion or variant according to claim 53.

81. A formulation comprising at least one COPD-related human Ig derived protein or specified portion or variant according to claim 53, and at least one selected from sterile water, sterile buffered water, or at least one preservative selected from the group consisting of phenol, m-cresol, p-cresol, o-cresol, chlorocresol, benzyl alcohol, alkylparaben, benzalkonium chloride, benzethonium chloride, sodium dehydroacetate and thimerosal, or mixtures thereof in an aqueous diluent.

82. A formulation of claim 81, wherein the concentration of COPD-related human Ig derived protein or specified portion or variant is about 0.1 mg/ml to about 100 mg/ml.

83. A formulation of claim 81, further comprising an isotonicity agent.

84. A formulation of claim 81, further comprising a physiologically acceptable buffer.

85. A formulation comprising at least one COPD-related human Ig derived protein or specified portion or variant according to claim 53 in lyophilized form in a first container, and an optional second container comprising at least one of sterile water, sterile buffered water, or at least one preservative selected from the group consisting of phenol, m-cresol, p-cresol, o-cresol, chlorocresol, benzyl alcohol, alkylparaben, benzalkonium chloride, benzethonium chloride, sodium dehydroacetate and thimerosal, or mixtures thereof in an aqueous diluent.

86. A formulation of claim 85, wherein the concentration of COPD-related human Ig derived protein or specified portion or variant is reconsitituted to a concentration of about 0.1 mg/ml to about 500 mg/ml.

87. A formulation of claim 85, further comprising an isotonicity agent.

88. A formulation of claim 85, further comprising a physiologically acceptable buffer.

89. A method of treating at least one COPD-related mediated condition, comprising administering to a patient in need thereof a formulation according to claim 85.

90. An article of manufacture for human pharmaceutical use, comprising packaging material and a container comprising a solution or a lyophilized form of at least one COPD-related human Ig derived protein or specified portion or variant according to claim 53.

91. The article of manufacture of claim 90, wherein said container is a glass or plastic container having a stopper for multi-use administration.

92. The article of manufacture of claim 90, wherein said container is a blister pack, capable of being punctured and used in intravenous, intramuscular, bolus, intraperitoneal, subcutaneous, respiratory, inhalation, nasal, vaginal, rectal, buccal, sublingual, intranasal, subdermal, or transdermal administration.

93. The article of manufacture of claim 90, wherein said container is a component of a intravenous, intramuscular, bolus, intraperitoneal, subcutaneous, respiratory, inhalation, nasal, vaginal, rectal, buccal, sublingual, intranasal, subdermal, or transdermal delivery device or system.

94. The article of manufacture of claim 90, wherein said container is a component of an injector or pen-injector device or system for intravenous, intramuscular, bolus, intraperitoneal, subcutaneous, respiratory, inhalation, nasal, vaginal, rectal, buccal, sublingual, intranasal, subdermal, or transdermal.

95. A method for preparing a formulation of at least one COPD-related human Ig derived protein or specified portion or variant, comprising admixing at least one COPD-related human Ig derived protein or specified portion or variant according to claim 53 in at least one buffer containing saline or a salt.

96. A method for producing at least one COPD-related human Ig derived protein or specified portion or variant according to claim 53, comprising providing a host cell, transgenic animal, transgenic plant or plant cell capable of expressing in recoverable amounts said human Ig derived protein or specified portion or variant.

97. A method according to claim 96, wherein said host cell is a mammalian cell, a plant cell or a yeast cell.

98. A method according to claim 96, wherein said transgenic animal is a mammal.

99. A method according to claim 98, wherein said transgenic mammal is selected from a goat, a cow, a sheep, a horse, and a non-human primate.

100. A transgenic animal or plant expressing at least one human Ig derived protein according to claim 53.

101. At least one COPD-related human Ig derived protein or specified portion or variant produced by a method according to claim 96.


SEQUENCE LISTING (17 sequences)

SEQ ID NO: 1 - protein, 157 aa, Homo sapiens [sequence omitted]

SEQ ID NO: 2 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 3 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 4 - protein, 99 aa, Homo sapiens
MTSKLAVALLAAFLISAALCEGAVLPRSAKELRCQCIKTYSKPFHPKFIKELRVIESGPHCANTEIIVKLSDGRELCLDPKENWVQRVVEKFLKRAENS

SEQ ID NO: 5 - protein, 1207 aa, Homo sapiens
MLLTLIILLPVVSKFSFVSLSAPQHWSCPEGTLAGNGNSTCVGPAPFLIFSHGNSIFRIDTEGTNYEQLVVDAGVSVIMDFHYNEKRIYWVDLERQLLQRVFLNGSRQERVCNIEKNVSGMAINWINEEVIWSNQQEGIITVTDMKGNNSHILLSALKYPANVAVDPVERFIFWSSEVAGSLYRADLDGVGVKALLETSEKITAVSLDVLDKRLFWIQYNREGSNSLICSCDYDGGSVHISKHPTQHNLFAMSLFGDRIFYSTWKMKTIWIANKHTGKDMVRINLHSSFVPLGELKVVHPLAQPKAEDDTWEPEQKLCKLRKGNCSSTVCGQDLQSHLCMCAEGYALSRDRKYCEDVNECAFWNHGCTLGCKNTPGSYYCTCPVGFVLLPDGKRCHQLVSCPRNVSECSHDCVLTSEGPLCFCPEGSVLERDGKTCSGCSSPDNGGCSQLCVPLSPVSWECDCFPGYDLQLDEKSCAASGPQPFLLFANSQDIRHMHFDGTDYGTLLSQQMGMVYALDHDPVENKIYFAHTALKWIERANMDGSQRERLIEEGVDVPEGLAVDWIGRRFYWTDRGKSLIGRSDLNGKRSKIITKENISQPRGIAVHPMAKRLFWTDTGINPRIESSSLQGLGRLVIASSDLIWPSGITIDFLTDKLYWCDAKQSVIEMANLDGSKRRRLTQNDVGHPFAVAVFEDYVWFSDWAMPSVIRVNKRTGKDRVRLQGSMLKPSSLVVVHPLAKPGADPCLYQNGGCEHICKKRLGTAWCSCREGFMKASDGKTCLALDGHQLLAGGEVDLKNQVTPLDILSKTRVSEDNITESQHMLVAEIMVSDQDDCAPVGCSMYARCISEGEDATCQCLKGFAGDGKLCSDIDECEMGVPVCPPASSKCINTEGGYVCRCSEGYQGDGIHCLDIDECQLGVHSCGENASCTNTEGGYTCMCAGRLSEPGLICPDSTPPPHLREDDHHYSVRNSDSECPLSHDGYCLHDGVCMYIEALDKYACNCVVGYIGERCQYRDLKWWELRHAGHGQQQKVIVVAVCVVVLVMLLLLSLWGAHYYRTQKLLSKNPKNPYEESSRDVRSRRPADTEDGMSSCPQPWFVVIKEHQDLKNGGQPVAGEDGQAADGSMQPTSWRQEPQLCGMGTEQGCWIPVSSDKGSCPQVMERSFHMPSYGTQTLEGGVEKPHSLLSANPLWQQRALDPPHQMELTQ

SEQ ID NO: 6 - protein, 235 aa, Homo sapiens
MALPVTALLLPLALLLHAARPSQFRVSPLDRTWNLGETVELKCQVLLSNPTSGCSWLFQPRGAAASPTFLLYLSQNKPKAAEGLDTQRFSGKRLGDTFVLTLSDFRRENEGYYFCSALSNSIMYFSHFVPVFLPAKPTTTPAPRPPTPAPTIASQPLSLRPEACRPAAGGAVHTRGLDFACDIYIWAPLAGTCGVLLLSLVITLYCNHRNRRRVCKCPRPVVKSGDKPSLSARYV

SEQ ID NO: 7 - protein, 45 aa, Homo sapiens
TQKDFTNKQRIGFWCPATKRHRSVMSTMWKNERRDTFNPGEFNGC

SEQ ID NO: 8 - protein, 37 aa, Homo sapiens [sequence omitted]

SEQ ID NO: 9 - protein, 210 aa, Homo sapiens
MRPRLWLLLAAQLTVLHGNSVLQQTPAYIKVQTNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQFYK

SEQ ID NO: 10 - protein, 246 aa, Homo sapiens
MRPRLWLLLAAQLTVLHGNSVLQQTPAYIKVQYNKMVMLSCEAKISLSNMRIYWLRQRQAPSSDSHHEFLALWDSAKGTIHGEEVEQEKIAVFRDASRFILNLTSVKPEDSGIYFCMIVGSPELTFGKGTQLSVVDFLPTTAQPTKKSTLKKRVCRLPRPETQKGPLCSPITLGLLVAGVLVLLVSLGVAIHLCCRRRRARLRFMKQKFNIVCLKISGFTTCCCFQILQISREYGFGVLLQKDIGQ

SEQ ID NO: 11 - protein, 80 aa, Homo sapiens
TQKGPLCSPITGLLVAGVLVLLVSLGVAIHLCCRRRRARLRFMKQPQGEGISGTFVPQCLHGYYSNTTTSQKLLNPWILK

SEQ ID NO: 12 - protein, 769 aa, Homo sapiens
MLGLRPPLLALVGLLSLGCVLSQECTKFKVSSCRECIESGPGCTWCQKLNFTGPGDPDSIRCDTRPQLLMRGCAADDIMDPTSLAETQEDHNGGQKQLSPQKVTLYLRPGQAAAFNVTFRRAKGYPIDLYYLMDLSYSMLDDLRNVKKLGGDLLRALNEITESGRIGFGSFVDKTVLPFVNTHPDKLRNPCPNKEKECQPPFAFRHVLKLTNNSNQFQTEVGKQLISGNLDAPEGGLDAMMQVAACPEEIGWRNVTRLLVFATDDGFHFAGDGKLGAILTPNDGRCHLEDNLYKRSNEFDYPSVGQLAHKLAENNIQPIFAVTSRMVKTYEKLTEIIPKSAVGELSEDSSNVVHLIKNAYNKLSSRVFLDHNALPDTLKVTYDSFCSNGVTHRNQPRGDCDGVQINVPITFQVKVTATECIQEQSFVIRALGFTDIVTVQVLPQCECRCRDQSRDRSLCHGKGFLECGICRCDTGYIGKNCECQTQGRSSQELEGSCRKDNNSIICSGLGDCVCGQCLCHTSDVPGKLIYGQYCECDTINCERYNGQVCGGPGRGLCFCGKCRCHPGFEGSACQCERTTEGCLNPRRVECSGRGRCRCNVCECHSGYQLPLCQECPGCPSPCGKYISCAECLKFEKGPFGKNCSAACPGLQLSNNPVKGRTCKERDSEGCWVAYTLEQQDGMDRYLIYVDESRECVAGPNIAAIVGGTVAGIVLIGILLLVIWKALIHLSDLREYRRFEKEKLKSQWNNDNPLFKSATTTVMNPKFAES

SEQ ID NO: 13 - protein, 455 aa, Homo sapiens
MGLSTVPDLLLPLVLLELLVGIYPSGVIGLVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTTVLLPLVIFFGLCLLSLLFIGLMYRYQRWKSKLYSIVCGKSTPEKEGELEGTTTKPLAPNPSFSPTPGFTPTLGFSPVPSSTFTSSSTYTPGDCPNFAAPRREVAPPYQGADPILATALASDPIPNPLQKWEDSAHKPQSLDTDDPATLYAVVENVPPLRWKEFVRRLGLSDHEIDRLELQNGRCLREAQYSMLATWRRRTPRREATLELLGRVLRDMDLLGCLEDIEEALCGPAALPPAPSLLR

SEQ ID NO: 14 - protein, 53 aa, Homo sapiens
TPWTESRSPPAENEVSTPMQALTTNKDDDNILFRDSANATSLPGSRRRGSCGL

SEQ ID NO: 15 - protein, 350 aa, Homo sapiens
MSNITDPQMWDFDDLNFTGMPPADEDYSPCMLETETLNKYVVIIAYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRHLVKFVCLGCWGLSMNLSLPFFLFRQAYHPNNSSPVCYEVLGNDTAKWRMVLRILPHTFGFIVPLFVMLFCYGFTLRTLFKAHMGQKHRAMRVIFAVVLIFLLCWLPYNLVLLADTLMRTQVIQESCERRNNIGRALDATEILGFLHSCLNPIIYAFIGQNFRHGFLKILAMHGLVSKEFLARHRVTSYTSSSVNVSSNL

SEQ ID NO: 16 - protein, 360 aa, Homo sapiens
MEDFNMESDSFEDFWKGEDLSNYSYSSTLPPFLLDAAPCEPESLEINKYFVVIIYALVFLLSLLGNSLVMLVILYSRVGRSVTDVYLLNLALADLLFALTLPIWAASKVNGWIFGTFLCKVVSLLKEVNFYSGILLLACISVDRYLAIVHATRTLTQKRYLVKFICLSIWGLSLLLALPVLLFRRTVYSSNVSPACYEDMGNNTANWRMLLRILPQSFGFIVPLLIMLFCYGFTLRTLFKAHMGQKHRAMRVIFAVVLIFLLCWLPYNLVLLADTLMRTQVIQETCERRNHIDRALDATEILGILHSCLNPLIYAFIGQKFRHGLLKILAIHGLISKDSLPKDSRPSFVGSSSGHTSTTL

SEQ ID NO: 17 - protein, 1210 aa, Homo sapiens
MRPSGTAGAALLALLAALCPASRALEEKKVCQGTSNKLTQLGTFEDHFLSLQRMFNNCEVVLGNLEITYVQRNYDLSFLKTIQEVAGYVLIALNTVERIPLENLQIIRGNMYYENSYALAVLSNYDANKTGLKELPMRNLQEILHGAVRFSNNPALCNVESIQWRDIVSSDFLSNMSMDFQNHLGSCQKCDPSCPNGSCWGAGEENCQKLTKIICAQQCSGRCRGKSPSDCCHNQCAAGCTGPRESDCLVCRKFRDEATCKDTCPPLMLYNPTTYQMDVNPEGKYSFGATCVKKCPRNYVVTDHGSCVRACGADSYEMEEDGVRKCKKCEGPCRKVCNGIGIGEFKDSLSINATNIKHFKNCTSISGDLHILPVAFRGDSFTHTPPLDPQELDILKTVKEITGFLLIQAWPENRTDLHAFENLEIIRGRTKQHGQFSLAVVSLNITSLGLRSLKEISDGDVIISGNKNLCYANTINWKKLFGTSGQKTKIISNRGENSCKATGQVCHALCSPEGCWGPEPRDCVSCRNVSRGRECVDKCKLLEGEPREFVENSECIQCHPECLPQAMNITCTGRGPDNCIQCAHYIDGPHCVKTCPAGVMGENNTLVWKYADAGHVCHLCHPNCTYGCTGPGLEGCPTNGPKIPSIATGMVGALLLLLVVALGIGLFMRRRHIVRKRTLRRLLQERELVEPLTPSGEAPNQALLRILKETEFKKIKVLGSGAFGTVYKGLWIPEGEKVKIPVAIKELREATSPKANKEILDEAYVMASVDNPHVCRLLGICLTSTVQLITQLMPFGCLLDYVREHKDNIGSQYLLNWCVQIAKGMNYLEDRRLVHRDLAARNVLVKTPQHVKITDFGLAKLLGAEEKEYHAEGGKVPIKWMALESILHRIYTHQSDVWSYGVTVWELMTFGSKPYDGIPASEISSILEKGERLPQPPICTIDVYMIMVKCWMIDADSRPKFRELIIEFSKMARDPQRYLVIQGDERMHLPSPTDSNFYRALMDEEDMDDVVDADEYLIPQQGFFSSPSTSRTPLLSSLSATSNNSTVACIDRNGLQSCPIKEDSFLQRYSSDPTGALTEDSIDDTFLPVPEYINQSVPKRPAGSVQNPVYHNQPLNPAPSRDPHYQDPHSTAVGNPEYLNTVQPTCVNSTFDSPAHWAQKGSHQISLDNPDYQQDFFPKEAKPNGIFKGSTAENAEYLRVAPQSSEFIGA